SEQUENCE LISTING (87 sequences)

SEQ ID NO: 1 - protein, 199 aa, Clostridium sp.
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 2 - DNA, 597 bp, Clostridium sp.
  source 1..597: mol_type = other DNA
gaaggcatcaaaagcaacatctccctcctgaaagacgaactccgggggcagattagccacattagtcacgaatacctctccctcatcgacctggctttcgatagcaagcagaacaggctctttgagatgaaagtgctggaactgctcgtcaatgagtacgggttcaagggtcgacacctcggcggatctaggaaaccagacggcatcgtgtatagtaccacactggaagacaactttgggatcattgtggataccaaggcatactctgagggttatagtctgcccatttcacaggccgacgagatggaacggtacgtgcgcgagaactcaaatagagatgaggaagtcaaccctaacaagtggtgggagaacttctctgaggaagtgaagaaatactacttcgtctttatcagcgggtccttcaagggtaaatttgaggaacagctcaggagactgagcatgactaccggcgtgaatggcagcgccgtcaacgtggtcaatctgctcctgggcgctgaaaagattcggagcggagagatgaccatcgaagagctggagagggcaatgtttaataatagcgagtttatcctgaaatac

SEQ ID NO: 3 - DNA, 5866 bp, synthetic construct
  misc_feature 1..5866: note = pCAG-TAL-nuclease
  source 1..5866: mol_type = other DNA
ggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttatgagacgacgtctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaacgatcacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatga

SEQ ID NO: 4 - protein, 176 aa, synthetic construct
  REGION 1..176: note = N-terminal peptide
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLN

SEQ ID NO: 5 - protein, 78 aa, synthetic construct
  REGION 1..78: note = C-terminal peptide
LTPQQVVAIASNGGGRPALESIVAQLSRPDPALARSALTNDHLVALACLGGRPALDAVKKGLPHAPALIKRTNRRIPE

SEQ ID NO: 6 - protein, 408 aa, synthetic construct
  REGION 1..408: note = ArtTal1
LTPEQVVAIASNIGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNGGGKQALETVQRLLPVLCQAHG

SEQ ID NO: 7 - DNA, 7067 bp, synthetic construct
  misc_feature 1..7067: note = pCAG-ArtTal1-nuclease
  source 1..7067: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttactccagaacaggtcgtcgcaatcgcaagtaacatcggcggaaaacaggccctcgaaaccgtccagagactcctccccgtgctgtgccaggcccacggactgaccccacagcaggtggtcgccatcgctagcaacggcggagggaagcaggctctggagaccgtgcagaggctgctccccgtcctgtgccaggcacatgggctcacacctcagcaggtggtcgcaattgcctccaatggtggcggaaaacaggccctggaaactgtgcagagactgctccccgtgctgtgccaggctcacggtctcacaccccagcaggtggtcgctatcgcatctcatgacgggggcaagcaggcactggagacagtgcagcggctgctccctgtcctgtgccaggcccacggactcactcctcagcaggtcgtcgccattgctagtaacggcggagggaaacaggctctggaaaccgtgcagcgcctgctccccgtgctgtgccaagcccacggcctgaccccccagcaggtggtcgcaatcgcctcaaacaatggtggcaagcaggccctggagactgtgcagcgactgctcccagtgctgtgccaggcccatggactcacaccacagcaggtcgtcgctattgcaagcaacaatggagggaaacaggcactggaaacagtccagaggctgctccccgtgctgtgccaagcgcatggactcactccccagcaggtcgtcgccatcgcttccaataacggcggcaagcaggccctggagaccgtccagagactgctccccgtgctgtgccaagctcacggactcacacctgagcaggtcgtggcaatcgcctctaacattggagggaaacaggccctggaaactgtacagcggctgctccccgtgctgtgccaagcacacggactcactccacagcaggtcgtggccattgcaagtcatgacggaggcaagcaggccctggaaacagtgcagcgcctgctccctgtgctgtgccaggctcatggtctgactcctcagcaggtggtggccatcgcttccaacaatggagggaagcaggccctggagaccgtacagagactgctccccgtgctgtgccaagcgcacggtctgacccctcagcaggtcgtcgcaatcgccagcaatggcgggggcaagcaggctctcgaaaccgtccagcggctcctcccagtcctctgtcaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaacgatcacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccggattc

SEQ ID NO: 8 - protein, 476 aa, synthetic construct
  REGION 1..476: note = TalRab2
LTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASHDGGKQALETVQRLLPVLCQAHGLTPQQVVAIASHDGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQRLLPVLCQAHGLTPQQVVAIASNNGGKQALETVQRLLPVLCQAHGLTPQQVVAIASNGGGKQALETVQRLLPVLCQAHG

SEQ ID NO: 9 - DNA, 7271 bp, synthetic construct
  misc_feature 1..7271: note = pCAG-TalRab2-nuclease
  source 1..7271: mol_type = other DNA
ggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatctgacaccccagcaggtggtggccattgctagcaacaatgggggcaagcaggctctggagacagtgcagcgcctgctgcctgtgctgtgccaggctcacggactgactccacagcaggtggtggccatcgcttccaacaatggagggaaacaggctctggaaacagtgcagaggctgctgcccgtgctgtgccaggctcatggactgacacctcagcaggtcgtcgccattgcttctaacggcggagggaagcaggctctggagactgtgcagagactgctgccagtgctgtgccaggcccatggactgacccctcagcaggtcgtggctatcgctagtaacaatggcggaaaacaggctctggaaactgtgcagcggctgctccccgtgctgtgccaggcccacggcctcactccacagcaggtcgtcgctatcgcctctaataacgggggcaagcaggctctggagacagtacagcgcctgttacccgtgctgtgccaggcacacggcctcacacctcagcaggtcgtggcaatcgcttcccatgacggagggaaacaggctctggaaacggtccagaggctgctccccgtgctgtgccaagctcacggcctcacccctcagcaggtggtcgctattgcttctcatgatggcggaaagcaggctctggagaccgtgcagagactgctccctgtgctgtgccaagcccacggcctgactccacagcaggtcgtggccatcgctagtcatgacgggggcaaacaggctctggaaacagtacagcggctgttacccgtgctgtgccaagcccatggcctcacacctcagcaagtcgtcgctatcgctagcaacaatggagggaagcaggctctggagacggtgcagcgcctgctcccagtgctgtgccaagctcatggcctcacccctcagcaagtcgtcgcaattgcttccaataacggcggaaaacaggctctggaaaccgtccagaggctgctgcccgtgctgtgccaagcacatggcttaactccacagcaagtggtggccattgcttctaatgggggcggaaagcaggccctggagacagtccagagactgttgcccgtgctgtgccaagcgcatggactgacacctgaacaggtcgtcgctatcgctagtaatattgggggcaaacaggccctggaaacagtgcagcggctgcttcccgtgctgtgccaggcgcatggactcacaccccagcaggtcgtcgcaatcgcctctaataacggagggaagcaggccctggaaaccgtgcagagactgttacctgtgctgtgccaggcacatggtctgacaccacagcaggtggtcgcaattgctagcaatggcggagggaagcaggccctggagactgtccagagactgctacccgtgctgtgccaagcgcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaacgatcacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatga

SEQ ID NO: 10 - protein, 583 aa, Flavobacterium sp.
MFLSMVSKIRTFGWVQNPGKFENLKRVVQVFDRNSKVHNEVKNIKIPTLVKESKIQKELVAIMNQHDLIYTYKELVGTGTSIRSEAPCDAIIQATIADQGNKKGYIDNWSSDGFLRWAHALGFIEYINKSDSFVITDVGLAYSKSADGSAIEKEILIEAISSYPPAIRILTLLEDGQHLTKFDLGKNLGFSGESGFTSLPEGILLDTLANAMPKDKGEIRNNWEGSSDKYARMIGGWLDKLGLVKQGKKEFIIPTLGKPDNKEFISHAFKITGEGLKVLRRAKGSTKFTRVPKRVYWEMLATNLTDKEYVRTRRALILEILIKAGSLKIEQIQDNLKKLGFDEVIETIENDIKGLINTGIFIEIKGRFYQLKDHILQFVIPNRGVTKQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 11 - protein, 558 aa, Acinetobacter lwoffii
MSTWLLGNTTVRSPFRLIDGLKVFALTNGDIRGTKEKELVFCKALVEGGIISASFEAEDTSGFSDTTYSVGRKWRSALEKLGFIEQFNQIYILTENGRNLLNSQTLQSDQECYLRSLILYSYKAENSDNPGGFFSPLMLTLHIMKELEIRTGSSRISFQEMAAVIQLTFSYLDINQSVNEILTIRSNRQASLSKKKFDRELYESKSSKAKIKAPSIKDYADTNLRYLKSTGLFTASGKGICFIDDKKIVIDKLIAMYGTFDISQSDLKIQKGAPLPTDHKETNILLVEQLEETLNRNRILFEKNSSIAQAPIGEIKNYRYHLEELLFENNEKKFAENQKNEWDEILAYMDLLISPKPISIEIADKEISIPSGERPAYFEWVLWRAFLALNHLIIEPQQCRRFKVDQDFKPIHNAPGGGADVIFEYENFKILGEVTLTSNSRQEAAEGEPVRRHIAVETVNTPDKDVYGLFLALTIDTNTAETFRHGAWYHQEELMDVKILPLTLESFKKYLESLRKKNQVETGIFDLKKMMDESLKLRETLTAPQWKNEITNKFARPI

SEQ ID NO: 12 - protein, 556 aa, Micrococcus lylae
MASLSKTKHLFGFTSPRTIEKIIPELDILSQQFSGKVWGENQINFFDAIFNSDFYEGTTYPQDPALAARDRITRAPKALGFIQLKPVIQLTKAGNQLVNQKRLPELFTKQLLKFQLPSPYHTQSPTVNFNVRPYLELLRLINELGSISKTEIALFFLQLVNYNKFDEIKNKILKFRETRKNNRSVSWKTYVSQEFEKQISIIFADEVTAKNFRTRESSDESFKKFVKTKEGNMKDYADAFFRYIRGTQLVTIDKNLHLKISSLKQDSVDFLLKNTDRNALNLSLMEYENYLFDPDQLIVLEDNSGLINSKIKQLDDSINVESLKIDDAKDLLNDLEIQRKAKTIEDTVNHLKLRSDIEDILDVFAKIKKRDVPDVPLFLEWNIWRAFAALNHTQAIEGNFIVDLDGMPLNTAPGKKPDIEINYGSFSCIVEVTMSSGETQFNMEGSSVPRHYGDLVRKVDHDAYCIFIAPKVAPGTKAHFFNLNRLSTKHYGGKTKIIPMSLDDFICFLQVGITHNFQDINKLKNWLDNLINFNLESEDEEIWFEEIISKISTWAI

SEQ ID NO: 13 - protein, 323 aa, Streptomyces sp.
MNSSDGIDGTVASIDTARALLKRFGFDAQRYNVRSAVTLLALAGLKPGDRWVDSTTPRLGVQKIMDWSGEHWAKPYATGSREDFRKKTLRQWVDNGFAVLNADNLNIATNSQLNEYCLSDEALQALRAYGTEGFEESLVVFLDEASKAVKARAEALQAAMISVDLPGGEEFLLSPAGQNPLLKKMVEEFVPRFAPRSTVLYLGDTRGKHSLFEREIFEEVLGLTFDPHGRMPDLILHDEVRGWLFLMEAVKSKGPFDEERHRSLQELFVTPSAGLIFVNCFENRESMRQWLPELAWETEAWVAEDPDHLIHLNGSRFLGPYER

SEQ ID NO: 14 - protein, 323 aa, Streptomyces diastaticus
MTNSNDIDETAATIDTARALLKSFGFEAQRHNVRSAVTLLALAGLKPGDHWADSTTPRLGVQKIMDWSGAYWAKPYATGSREDFRKKTLRQWVDNGFAVLNPDNLNIATNSQLNEYCLSDEAAQAIRSYGTDAFESALVDFLSKASDTVRARAEALRAAMISVDLADGDEFLLSPAGQNPLLKKMVEEFMPRFAPGAKVLYIGDWRGKHTRFEKRIFEETLGLTFDPHGRMPDLVLHDKVRKWLFLMEAVKSKGPFDEERHRTLRELFATPVAGLVFVNCFENREAMRQWLPELAWETEAWVADDPDHLIHLNGSRFLGPYER

SEQ ID NO: 15 - protein, 602 aa, Streptococcus sanguinis
MTISINEYSDLNNLAFGLGQDVSQDLKELVKVASIFMPDSKIHKWLIDTRLEEVVTDLNLRYELKSVITNTPISVTWKQLTGTRTKREANSLVQAVFPGQCSRLAIVDWAAKNYVSVAVAFGLLKFHRADKTFTISEIGIQAVKLYDSEELAELDKFLYERLLEYPYAAWLIRLLGNQPSKQFSKFDLGEHFGFIDELGFETAPIEIFLNGLAQAEIDGDKTAAQKIKSNFESTSDKYMRWLAGVLVTAGLATSTTKKVTHTYKNRKFELTLGTVYQITAKGLTALKEVNGKSRYPRSRKRVMWEFLATKDKEAIAKKTSRSLMLKHLTEKKNPIQAEVIATLINTDYPTLEITPEEVIDDCIGLNRIGIEILIDGDKLTLNDKLFDFEIPVQKDVVLEKSDIEKFKNQLRTELTNIDHSYLKGIDIASKKKTSNVENTEFEAISTKIFTDELGFSGKHLGGSNKPDGLLWDDDCAIILDSKAYSEGFPLTASHTDAMGRYLRQFTERKEEIKPTWWDIAPEHLDNTYFAYVSGSFSGNYKEQLQKFRQDTNHLGGALEFVKLLLLANNYKTQKMSKKEVKKSILDYNISYEEYAPLLAEIE

SEQ ID NO: 16 - protein, 593 aa, Clostridium leptum
MIHLIPTEAKRFRTFGWVQDPSDFRSLCDVVAIFDETSLKHQELAGQVIPALVEERDGRQRLLDALNQRPLRISYTDLVGTSFTPRSAARCNGIVQAAVRGQVRPFIGDWPADNFVRWAHALGFLRYGYQGDAFELTETGKALAQARTQGEELNSQEKELLTSAVLAYPPAVRILSLLGEGEGAHLTKFELGKQLGFVGEDGFTSLPQTVLVRSLASSKDAKEKNKMKTDWDGSSDKYARMIAKWLEKLGLVKQEAKPVTVTLAGRKYTESIGQSYVITGLGITALNRTLGKSRHKRIPKNVSFEMMATKGDDREYLRTRRTCVLKAVSEGKGRVSYTEIQKYLEALGLQEDEATIRDDVQGLIHIGLNIAAGERECVWKDEINDLILPVPKKLAKSSQSETKEKLREKLRNLPHEYLSLVDLAYDSKQNRLFEMKVIELLTEECGFQGLHLGGSRRPDGVLYTAGLTDNYGIILDTKAYSSGYSLPIAQADEMERYVRENQTRDELVNPNQWWENFENGLGTFYFLFVAGHFNGNVQAQLERISRNTGVLGAAASISQLLLLADAIRGGRMDRERLRHLMFQNEEFLLEQEL

SEQ ID NO: 17 - protein, 587 aa, Clostridium sp.
MINIIDVNNKTIRTFGWVQNPSNFESLKKVVAIFDNTSKTYNELKDKKIKKLVDERDGQKELLNALNANPLKIKYCNLVGTSFTPRSSARCNGIVQATVKGQRKEFIDDWSSDNFVRWAHALGFIKYNYDTDTFEITDVGRKYVQSEDDSNEESTILEEAMLSYPPVARVLTLLSNGEHLTKYEIGKKLGFVGEAGFTSLPLNVLIMTLATTDEPKEKNKIKTDWDGSSDKYARMISGWLVKLGLLVQRPKLVTVDFGGELYSETIGHAYMITDRGLKAVRRLLGINKVARVSKNVFWEMLATKGIDKNYIRTRRAYILKILIESNKVLTLEDIKGKLKLASINESINTIKDDINGLINTGINIKSETTGYKIYDSINDFIIPKTGDTEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 18 - protein, 589 aa, Peptoniphilus sp.
MAERTLGWIQNPSSFENLKNVVSVFDKNSDIYKEILNTKLPKLVKDLDLQNKLISELEKDPLEMDYVLLKGHGIKSGQKRADAECSGIVQAAITTQGGRAYTDDWTADGFLRWGISIGLLDYDTEKDTVSITKLGEKFVKSNSEDSDKEILISAFLSYPPAVRILTLLENGDHLTKFELGKQLGGLGEAGFTSIPQDLYIQAIELAADKDKASIRSNTEGSADKYARMISGWLSKVGLIQRIGKEVSTKIGDVEYKVNIGHSFRITLNGIKELKRAMGLSSYPKTDKIVYWQMLATKGKDRDYIRNRRGYIIKAINNRERNLEDIKAYLLENNIDESITTIEDELKVIEAMGLSFKHSRNGYVIDDNIIKLEIPRTKISKTNVLELKDKVRDKLKYVDHRYLALIDLAYDGTANRDFEIQTIDLLINELKFKGVRLGESRKPDGIISYNINGVIIDNKAYSTGYNLPINQADEMIRYIEENQTRDEKINSNKWWESFDDKVKDFNYLFVSSFFKGNFKNNLKHIANRTGVSGGAINVENLLYFAEELKAGRLSYVDSFKMYDNDEIYVGDFSDYSYVKFAAEEEGEYLT

SEQ ID NO: 19 - protein, 279 aa, Acinetobacter lwoffii
KETNILLVEQLEETLNRNRILFEKNSSIAQAPIGEIKNYRYHLEELLFENNEKKFAENQKNEWDEILAYMDLLISPKPISIEIADKEISIPSGERPAYFEWVLWRAFLALNHLIIEPQQCRRFKVDQDFKPIHNAPGGGADVIFEYENFKILGEVTLTSNSRQEAAEGEPVRRHIAVETVNTPDKDVYGLFLALTIDTNTAETFRHGAWYHQEELMDVKILPLTLESFKKYLESLRKKNQVETGIFDLKKMMDESLKLRETLTAPQWKNEITNKFARPI

SEQ ID NO: 20 - protein, 201 aa, Clostridium leptum
KLAKSSQSETKEKLREKLRNLPHEYLSLVDLAYDSKQNRLFEMKVIELLTEECGFQGLHLGGSRRPDGVLYTAGLTDNYGIILDTKAYSSGYSLPIAQADEMERYVRENQTRDELVNPNQWWENFENGLGTFYFLFVAGHFNGNVQAQLERISRNTGVLGAAASISQLLLLADAIRGGRMDRERLRHLMFQNEEFLLEQEL

SEQ ID NO: 21 - protein, 250 aa, Micrococcus lylae
INSKIKQLDDSINVESLKIDDAKDLLNDLEIQRKAKTIEDTVNHLKLRSDIEDILDVFAKIKKRDVPDVPLFLEWNIWRAFAALNHTQAIEGNFIVDLDGMPLNTAPGKKPDIEINYGSFSCIVEVTMSSGETQFNMEGSSVPRHYGDLVRKVDHDAYCIFIAPKVAPGTKAHFFNLNRLSTKHYGGKTKIIPMSLDDFICFLQVGITHNFQDINKLKNWLDNLINFNLESEDEEIWFEEIISKISTWAI

SEQ ID NO: 22 - protein, 213 aa, Peptoniphilus sp.
KISKTNVLELKDKVRDKLKYVDHRYLALIDLAYDGTANRDFEIQTIDLLINELKFKGVRLGESRKPDGIISYNINGVIIDNKAYSTGYNLPINQADEMIRYIEENQTRDEKINSNKWWESFDDKVKDFNYLFVSSFFKGNFKNNLKHIANRTGVSGGAINVENLLYFAEELKAGRLSYVDSFKMYDNDEIYVGDFSDYSYVKFAAEEEGEYLT

SEQ ID NO: 23 - protein, 163 aa, Streptomyces sp.
ISVDLPGGEEFLLSPAGQNPLLKKMVEEFVPRFAPRSTVLYLGDTRGKHSLFEREIFEEVLGLTFDPHGRMPDLILHDEVRGWLFLMEAVKSKGPFDEERHRSLQELFVTPSAGLIFVNCFENRESMRQWLPELAWETEAWVAEDPDHLIHLNGSRFLGPYER

SEQ ID NO: 24 - protein, 163 aa, Streptomyces diastaticus
ISVDLADGDEFLLSPAGQNPLLKKMVEEFMPRFAPGAKVLYIGDWRGKHTRFEKRIFEETLGLTFDPHGRMPDLVLHDKVRKWLFLMEAVKSKGPFDEERHRTLRELFATPVAGLVFVNCFENREAMRQWLPELAWETEAWVADDPDHLIHLNGSRFLGPYER

SEQ ID NO: 25 - protein, 208 aa, Streptococcus sanguinis
DVVLEKSDIEKFKNQLRTELTNIDHSYLKGIDIASKKKTSNVENTEFEAISTKIFTDELGFSGKHLGGSNKPDGLLWDDDCAIILDSKAYSEGFPLTASHTDAMGRYLRQFTERKEEIKPTWWDIAPEHLDNTYFAYVSGSFSGNYKEQLQKFRQDTNHLGGALEFVKLLLLANNYKTQKMSKKEVKKSILDYNISYEEYAPLLAEIE

SEQ ID NO: 26 - protein, 196 aa, Flavobacterium sp.
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 27 - DNA, 7903 bp, synthetic construct
  misc_feature 1..7903: note = pCAG-ArtTal1-AlwI
  source 1..7903: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttactccagaacaggtcgtcgcaatcgcaagtaacatcggcggaaaacaggccctcgaaaccgtccagagactcctccccgtgctgtgccaggcccacggactgaccccacagcaggtggtcgccatcgctagcaacggcggagggaagcaggctctggagaccgtgcagaggctgctccccgtcctgtgccaggcacatgggctcacacctcagcaggtggtcgcaattgcctccaatggtggcggaaaacaggccctggaaactgtgcagagactgctccccgtgctgtgccaggctcacggtctcacaccccagcaggtggtcgctatcgcatctcatgacgggggcaagcaggcactggagacagtgcagcggctgctccctgtcctgtgccaggcccacggactcactcctcagcaggtcgtcgccattgctagtaacggcggagggaaacaggctctggaaaccgtgcagcgcctgctccccgtgctgtgccaagcccacggcctgaccccccagcaggtggtcgcaatcgcctcaaacaatggtggcaagcaggccctggagactgtgcagcgactgctcccagtgctgtgccaggcccatggactcacaccacagcaggtcgtcgctattgcaagcaacaatggagggaaacaggcactggaaacagtccagaggctgctccccgtgctgtgccaagcgcatggactcactccccagcaggtcgtcgccatcgcttccaataacggcggcaagcaggccctggagaccgtccagagactgctccccgtgctgtgccaagctcacggactcacacctgagcaggtcgtggcaatcgcctctaacattggagggaaacaggccctggaaactgtacagcggctgctccccgtgctgtgccaagcacacggactcactccacagcaggtcgtggccattgcaagtcatgacggaggcaagcaggccctggaaacagtgcagcgcctgctccctgtgctgtgccaggctcatggtctgactcctcagcaggtggtggccatcgcttccaacaatggagggaagcaggccctggagaccgtacagagactgctccccgtgctgtgccaagcgcacggtctgacccctcagcaggtcgtcgcaatcgccagcaatggcgggggcaagcaggctctcgaaaccgtccagcggctcctcccagtcctctgtcaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaacaaagagactaatatcctcctcgtcgagcagctggaagagaccctcaatcgcaatcgcattctgtttgaaaagaactcctcaatcgcacaggccccaattggcgagatcaagaactaccggtatcacctggaggaactgctcttcgagaacaatgaaaagaaatttgcagagaaccagaaaaatgagtgggacgaaattctggcctacatggatctgctcatctcacccaagcctatcagcattgagatcgctgacaaagaaatttctatcccaagtggggagcgacccgcatatttcgaatgggtgctgtggagggcatttctggccctcaaccacctgatcattgagccccagcagtgcaggagattcaaggtcgaccaggacttcaagcctatccataatgctccaggcggaggggcagatgtgattttcgagtacgaaaactttaagatcctgggcgaggtcaccctcacaagcaattcccgacaggaagcagctgagggagaacccgtgcggcgccatattgccgtggagacagtcaacactcctgacaaggatgtctatggactgttcctcgctctgaccatcgacactaataccgccgagacatttcgacacggggcttggtatcaccaggaggaactgatggatgtgaagattctccccctgactctcgagtccttcaagaagtatctggaatctctcagaaagaaaaatcaggtggagacaggaatctttgacctgaagaaaatgatggatgaaagcctgaagctccgggaaaccctgaccgcaccccagtggaaaaatgaaatcacaaacaaattcgccagaccaatctgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccggattc

SEQ ID NO: 28 - DNA, 7669 bp, synthetic construct
  misc_feature 1..7669: note = pCAG-ArtTal1-CLEDORF
  source 1..7669: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttactccagaacaggtcgtcgcaatcgcaagtaacatcggcggaaaacaggccctcgaaaccgtccagagactcctccccgtgctgtgccaggcccacggactgaccccacagcaggtggtcgccatcgctagcaacggcggagggaagcaggctctggagaccgtgcagaggctgctccccgtcctgtgccaggcacatgggctcacacctcagcaggtggtcgcaattgcctccaatggtggcggaaaacaggccctggaaactgtgcagagactgctccccgtgctgtgccaggctcacggtctcacaccccagcaggtggtcgctatcgcatctcatgacgggggcaagcaggcactggagacagtgcagcggctgctccctgtcctgtgccaggcccacggactcactcctcagcaggtcgtcgccattgctagtaacggcggagggaaacaggctctggaaaccgtgcagcgcctgctccccgtgctgtgccaagcccacggcctgaccccccagcaggtggtcgcaatcgcctcaaacaatggtggcaagcaggccctggagactgtgcagcgactgctcccagtgctgtgccaggcccatggactcacaccacagcaggtcgtcgctattgcaagcaacaatggagggaaacaggcactggaaacagtccagaggctgctccccgtgctgtgccaagcgcatggactcactccccagcaggtcgtcgccatcgcttccaataacggcggcaagcaggccctggagaccgtccagagactgctccccgtgctgtgccaagctcacggactcacacctgagcaggtcgtggcaatcgcctctaacattggagggaaacaggccctggaaactgtacagcggctgctccccgtgctgtgccaagcacacggactcactccacagcaggtcgtggccattgcaagtcatgacggaggcaagcaggccctggaaacagtgcagcgcctgctccctgtgctgtgccaggctcatggtctgactcctcagcaggtggtggccatcgcttccaacaatggagggaagcaggccctggagaccgtacagagactgctccccgtgctgtgccaagcgcacggtctgacccctcagcaggtcgtcgcaatcgccagcaatggcgggggcaagcaggctctcgaaaccgtccagcggctcctcccagtcctctgtcaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaacaagctcgcaaagtcaagccagtccgaaacaaaggaaaaactcagagaaaaactcagaaacctgccccatgaatacctgtccctcgtcgacctggcctacgattcaaagcagaaccgcctctttgagatgaaagtgatcgaactgctcacagaggaatgcgggttccagggtctgcacctcggcggaagcaggagaccagacggcgtcctgtacaccgccggactcacagacaactatgggatcattctggatactaaggcttacagctccggatattccctgcccattgcccaggctgacgagatggaacggtacgtgcgcgagaatcagactagagatgaactggtcaaccctaatcagtggtgggagaactttgaaaatggcctgggaaccttctattttctcttcgtggctgggcatttcaacggtaatgtccaggcacagctggagcgaatcagtaggaataccggcgtgctgggagccgctgcatctatcagtcagctgctcctgctcgcagacgccattagagggggtcggatggatagagagagactgcggcacctcatgtttcagaacgaagagtttctgctggaacaggagctgtgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccggattc

SEQ ID NO: 29 - DNA, 7663 bp, synthetic construct
  misc_feature 1..7663: note = pCAG-ArtTal1-Clo051
  source 1..7663: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttactccagaacaggtcgtcgcaatcgcaagtaacatcggcggaaaacaggccctcgaaaccgtccagagactcctccccgtgctgtgccaggcccacggactgaccccacagcaggtggtcgccatcgctagcaacggcggagggaagcaggctctggagaccgtgcagaggctgctccccgtcctgtgccaggcacatgggctcacacctcagcaggtggtcgcaattgcctccaatggtggcggaaaacaggccctggaaactgtgcagagactgctccccgtgctgtgccaggctcacggtctcacaccccagcaggtggtcgctatcgcatctcatgacgggggcaagcaggcactggagacagtgcagcggctgctccctgtcctgtgccaggcccacggactcactcctcagcaggtcgtcgccattgctagtaacggcggagggaaacaggctctggaaaccgtgcagcgcctgctccccgtgctgtgccaagcccacggcctgaccccccagcaggtggtcgcaatcgcctcaaacaatggtggcaagcaggccctggagactgtgcagcgactgctcccagtgctgtgccaggcccatggactcacaccacagcaggtcgtcgctattgcaagcaacaatggagggaaacaggcactggaaacagtccagaggctgctccccgtgctgtgccaagcgcatggactcactccccagcaggtcgtcgccatcgcttccaataacggcggcaagcaggccctggagaccgtccagagactgctccccgtgctgtgccaagctcacggactcacacctgagcaggtcgtggcaatcgcctctaacattggagggaaacaggccctggaaactgtacagcggctgctccccgtgctgtgccaagcacacggactcactccacagcaggtcgtggccattgcaagtcatgacggaggcaagcaggccctggaaacagtgcagcgcctgctccctgtgctgtgccaggctcatggtctgactcctcagcaggtggtggccatcgcttccaacaatggagggaagcaggccctggagaccgtacagagactgctccccgtgctgtgccaagcgcacggtctgacccctcagcaggtcgtcgcaatcgccagcaatggcgggggcaagcaggctctcgaaaccgtccagcggctcctcccagtcctctgtcaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaacgaaggcatcaaaagcaacatctccctcctgaaagacgaactccgggggcagattagccacattagtcacgaatacctctccctcatcgacctggctttcgatagcaagcagaacaggctctttgagatgaaagtgctggaactgctcgtcaatgagtacgggttcaagggtcgacacctcggcggatctaggaaaccagacggcatcgtgtatagtaccacactggaagacaactttgggatcattgtggataccaaggcatactctgagggttatagtctgcccatttcacaggccgacgagatggaacggtacgtgcgcgagaactcaaatagagatgaggaagtcaaccctaacaagtggtgggagaacttctctgaggaagtgaagaaatactacttcgtctttatcagcgggtccttcaagggtaaatttgaggaacagctcaggagactgagcatgactaccggcgtgaatggcagcgccgtcaacgtggtcaatctgctcctgggcgctgaaaagattcggagcggagagatgaccatcgaagagctggagagggcaatgtttaataatagcgagtttatcctgaaatactgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccggattc

SEQ ID NO: 30 - DNA, 7816 bp, synthetic construct
  misc_feature 1..7816: note = pCAG-ArtTal1-MlyI
  source 1..7816: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttactccagaacaggtcgtcgcaatcgcaagtaacatcggcggaaaacaggccctcgaaaccgtccagagactcctccccgtgctgtgccaggcccacggactgaccccacagcaggtggtcgccatcgctagcaacggcggagggaagcaggctctggagaccgtgcagaggctgctccccgtcctgtgccaggcacatgggctcacacctcagcaggtggtcgcaattgcctccaatggtggcggaaaacaggccctggaaactgtgcagagactgctccccgtgctgtgccaggctcacggtctcacaccccagcaggtggtcgctatcgcatctcatgacgggggcaagcaggcactggagacagtgcagcggctgctccctgtcctgtgccaggcccacggactcactcctcagcaggtcgtcgccattgctagtaacggcggagggaaacaggctctggaaaccgtgcagcgcctgctccccgtgctgtgccaagcccacggcctgaccccccagcaggtggtcgcaatcgcctcaaacaatggtggcaagcaggccctggagactgtgcagcgactgctcccagtgctgtgccaggcccatggactcacaccacagcaggtcgtcgctattgcaagcaacaatggagggaaacaggcactggaaacagtccagaggctgctccccgtgctgtgccaagcgcatggactcactccccagcaggtcgtcgccatcgcttccaataacggcggcaagcaggccctggagaccgtccagagactgctccccgtgctgtgccaagctcacggactcacacctgagcaggtcgtggcaatcgcctctaacattggagggaaacaggccctggaaactgtacagcggctgctccccgtgctgtgccaagcacacggactcactccacagcaggtcgtggccattgcaagtcatgacggaggcaagcaggccctggaaacagtgcagcgcctgctccctgtgctgtgccaggctcatggtctgactcctcagcaggtggtggccatcgcttccaacaatggagggaagcaggccctggagaccgtacagagactgctccccgtgctgtgccaagcgcacggtctgacccctcagcaggtcgtcgcaatcgccagcaatggcgggggcaagcaggctctcgaaaccgtccagcggctcctcccagtcctctgtcaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaacatcaatagcaagatcaagcagctggacgatagcatcaacgtggagtccctgaagattgacgatgccaaagatctgctgaatgacctggagatccagcggaaggctaaaaccattgaagatacagtgaaccacctgaagctgcgctccgacatcgaggatattctggacgtgttcgccaaaatcaagaaaagggatgtgcccgacgtgcctctgttcctggagtggaatatctggcgggcctttgccgctctgaatcatacccaggctatcgaagggaactttattgtggacctggatggcatgcccctgaatacagctccaggaaagaaacccgatatcgagattaactacggaagcttctcctgcatcgtggaagtgactatgagctccggggagacccagtttaacatggaaggctctagtgtgcctaggcactacggagacctggtgagaaaggtggaccatgatgcctattgtatcttcattgcccctaaggtggctccagggactaaagctcacttctttaacctgaataggctgtctacaaagcattatggcggaaagactaagatcattccaatgagtctggacgatttcatctgctttctgcaagtgggcattacccacaactttcaggatatcaacaagctgaaaaattggctggacaacctgattaacttcaatctggagtctgaagacgaggaaatctggtttgaggaaatcatttctaagatcagtacatgggccatttgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccggattc

SEQ ID NO: 31 - DNA, 7705 bp, synthetic construct
  misc_feature 1..7705: note = pCAG-ArtTal1-Pept071
  source 1..7705: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttactccagaacaggtcgtcgcaatcgcaagtaacatcggcggaaaacaggccctcgaaaccgtccagagactcctccccgtgctgtgccaggcccacggactgaccccacagcaggtggtcgccatcgctagcaacggcggagggaagcaggctctggagaccgtgcagaggctgctccccgtcctgtgccaggcacatgggctcacacctcagcaggtggtcgcaattgcctccaatggtggcggaaaacaggccctggaaactgtgcagagactgctccccgtgctgtgccaggctcacggtctcacaccccagcaggtggtcgctatcgcatctcatgacgggggcaagcaggcactggagacagtgcagcggctgctccctgtcctgtgccaggcccacggactcactcctcagcaggtcgtcgccattgctagtaacggcggagggaaacaggctctggaaaccgtgcagcgcctgctccccgtgctgtgccaagcccacggcctgaccccccagcaggtggtcgcaatcgcctcaaacaatggtggcaagcaggccctggagactgtgcagcgactgctcccagtgctgtgccaggcccatggactcacaccacagcaggtcgtcgctattgcaagcaacaatggagggaaacaggcactggaaacagtccagaggctgctccccgtgctgtgccaagcgcatggactcactccccagcaggtcgtcgccatcgcttccaataacggcggcaagcaggccctggagaccgtccagagactgctccccgtgctgtgccaagctcacggactcacacctgagcaggtcgtggcaatcgcctctaacattggagggaaacaggccctggaaactgtacagcggctgctccccgtgctgtgccaagcacacggactcactccacagcaggtcgtggccattgcaagtcatgacggaggcaagcaggccctggaaacagtgcagcgcctgctccctgtgctgtgccaggctcatggtctgactcctcagcaggtggtggccatcgcttccaacaatggagggaagcaggccctggagaccgtacagagactgctccccgtgctgtgccaagcgcacggtctgacccctcagcaggtcgtcgcaatcgccagcaatggcgggggcaagcaggctctcgaaaccgtccagcggctcctcccagtcctctgtcaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaacaagatcagcaaaaccaatgtgctggagctcaaggacaaagtccgagataagctgaaatacgtggaccacaggtatctggcactcatcgacctcgcctatgatgggaccgctaacagggacttcgaaatccagacaattgatctgctcattaatgagctgaagtttaaaggggtcaggctcggtgaaagtagaaagcccgacggcatcatttcatacaacatcaatggagtgatcattgataacaaggcttactctactggttataacctgcctattaatcaggccgacgagatgatccggtatattgaggaaaatcagacccgcgatgaaaaaatcaactccaataagtggtgggagtctttcgacgataaggtcaaagacttcaactacctgtttgtgagctccttctttaaggggaactttaaaaacaatctgaagcatatcgctaacagaacaggtgtcagcggcggagcaattaacgtggagaatctgctctacttcgcagaggaactgaaagccggccggctctcatatgtggatagctttaagatgtacgacaacgatgagatctatgtcggcgacttctctgattacagttatgtgaagtttgccgctgaggaagagggagaatacctgacttgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccggattc

SEQ ID NO: 32 - DNA, 7555 bp, synthetic construct
  misc_feature 1..7555: note = pCAG-ArtTal1-SbfI
  source 1..7555: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttactccagaacaggtcgtcgcaatcgcaagtaacatcggcggaaaacaggccctcgaaaccgtccagagactcctccccgtgctgtgccaggcccacggactgaccccacagcaggtggtcgccatcgctagcaacggcggagggaagcaggctctggagaccgtgcagaggctgctccccgtcctgtgccaggcacatgggctcacacctcagcaggtggtcgcaattgcctccaatggtggcggaaaacaggccctggaaactgtgcagagactgctccccgtgctgtgccaggctcacggtctcacaccccagcaggtggtcgctatcgcatctcatgacgggggcaagcaggcactggagacagtgcagcggctgctccctgtcctgtgccaggcccacggactcactcctcagcaggtcgtcgccattgctagtaacggcggagggaaacaggctctggaaaccgtgcagcgcctgctccccgtgctgtgccaagcccacggcctgaccccccagcaggtggtcgcaatcgcctcaaacaatggtggcaagcaggccctggagactgtgcagcgactgctcccagtgctgtgccaggcccatggactcacaccacagcaggtcgtcgctattgcaagcaacaatggagggaaacaggcactggaaacagtccagaggctgctccccgtgctgtgccaagcgcatggactcactccccagcaggtcgtcgccatcgcttccaataacggcggcaagcaggccctggagaccgtccagagactgctccccgtgctgtgccaagctcacggactcacacctgagcaggtcgtggcaatcgcctctaacattggagggaaacaggccctggaaactgtacagcggctgctccccgtgctgtgccaagcacacggactcactccacagcaggtcgtggccattgcaagtcatgacggaggcaagcaggccctggaaacagtgcagcgcctgctccctgtgctgtgccaggctcatggtctgactcctcagcaggtggtggccatcgcttccaacaatggagggaagcaggccctggagaccgtacagagactgctccccgtgctgtgccaagcgcacggtctgacccctcagcaggtcgtcgcaatcgccagcaatggcgggggcaagcaggctctcgaaaccgtccagcggctcctcccagtcctctgtcaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaacatctctgtggacctgccaggcggagaggaattcctgctgagtccagccggacagaaccccctgctgaagaaaatggtggaggaattcgtgccccggtttgctcctcgcagcaccgtgctgtacctgggggacacaaggggcaagcactccctgttcgagagagaaatctttgaggaagtgctgggcctgaccttcgatcctcacggacggatgccagacctgattctgcatgatgaggtgagggggtggctgttcctgatggaagccgtgaagtctaaaggcccctttgatgaggaaaggcatagaagcctgcaggagctgtttgtgactccttccgccggcctgatcttcgtgaactgctttgagaatagggaatctatgagacagtggctgcccgagctggcttgggagaccgaagcctgggtggctgaagaccctgatcacctgattcatctgaatggaagtcggtttctggggccatatgagcgctgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccggattc

SEQ ID NO: 33 - DNA, 7555 bp, synthetic construct
  misc_feature 1..7555: note = pCAG-ArtTal1-Sda
  source 1..7555: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttactccagaacaggtcgtcgcaatcgcaagtaacatcggcggaaaacaggccctcgaaaccgtccagagactcctccccgtgctgtgccaggcccacggactgaccccacagcaggtggtcgccatcgctagcaacggcggagggaagcaggctctggagaccgtgcagaggctgctccccgtcctgtgccaggcacatgggctcacacctcagcaggtggtcgcaattgcctccaatggtggcggaaaacaggccctggaaactgtgcagagactgctccccgtgctgtgccaggctcacggtctcacaccccagcaggtggtcgctatcgcatctcatgacgggggcaagcaggcactggagacagtgcagcggctgctccctgtcctgtgccaggcccacggactcactcctcagcaggtcgtcgccattgctagtaacggcggagggaaacaggctctggaaaccgtgcagcgcctgctccccgtgctgtgccaagcccacggcctgaccccccagcaggtggtcgcaatcgcctcaaacaatggtggcaagcaggccctggagactgtgcagcgactgctcccagtgctgtgccaggcccatggactcacaccacagcaggtcgtcgctattgcaagcaacaatggagggaaacaggcactggaaacagtccagaggctgctccccgtgctgtgccaagcgcatggactcactccccagcaggtcgtcgccatcgcttccaataacggcggcaagcaggccctggagaccgtccagagactgctccccgtgctgtgccaagctcacggactcacacctgagcaggtcgtggcaatcgcctctaacattggagggaaacaggccctggaaactgtacagcggctgctccccgtgctgtgccaagcacacggactcactccacagcaggtcgtggccattgcaagtcatgacggaggcaagcaggccctggaaacagtgcagcgcctgctccctgtgctgtgccaggctcatggtctgactcctcagcaggtggtggccatcgcttccaacaatggagggaagcaggccctggagaccgtacagagactgctccccgtgctgtgccaagcgcacggtctgacccctcagcaggtcgtcgcaatcgccagcaatggcgggggcaagcaggctctcgaaaccgtccagcggctcctcccagtcctctgtcaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaacattagcgtggacctcgccgatggagatgagttcctgctgagccccgctggacagaatcctctgctgaaaaagatggtggaagaatttatgccacgattcgcacctggagctaaggtgctgtacatcggcgactggcgaggaaagcacacacggttcgagaaacgcatttttgaggaaaccctggggctcacatttgatccacacggtagaatgcccgacctggtgctccatgataaggtccggaaatggctgttcctcatggaggccgtgaagagcaaaggcccttttgacgaggaaaggcatagaactctgcgggaactcttcgctaccccagtcgcaggactggtgttcgtcaactgctttgagaatcgagaagccatgaggcagtggctgcccgagctcgcttgggagaccgaagcatgggtggccgacgaccctgaccacctgatccacctcaacgggagcagattcctgggaccctatgaaagatgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccggattc

SEQ ID NO: 34 - DNA, 7690 bp, synthetic construct
  misc_feature 1..7690: note = pCAG-ArtTal1-StsI
  source 1..7690: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttactccagaacaggtcgtcgcaatcgcaagtaacatcggcggaaaacaggccctcgaaaccgtccagagactcctccccgtgctgtgccaggcccacggactgaccccacagcaggtggtcgccatcgctagcaacggcggagggaagcaggctctggagaccgtgcagaggctgctccccgtcctgtgccaggcacatgggctcacacctcagcaggtggtcgcaattgcctccaatggtggcggaaaacaggccctggaaactgtgcagagactgctccccgtgctgtgccaggctcacggtctcacaccccagcaggtggtcgctatcgcatctcatgacgggggcaagcaggcactggagacagtgcagcggctgctccctgtcctgtgccaggcccacggactcactcctcagcaggtcgtcgccattgctagtaacggcggagggaaacaggctctggaaaccgtgcagcgcctgctccccgtgctgtgccaagcccacggcctgaccccccagcaggtggtcgcaatcgcctcaaacaatggtggcaagcaggccctggagactgtgcagcgactgctcccagtgctgtgccaggcccatggactcacaccacagcaggtcgtcgctattgcaagcaacaatggagggaaacaggcactggaaacagtccagaggctgctccccgtgctgtgccaagcgcatggactcactccccagcaggtcgtcgccatcgcttccaataacggcggcaagcaggccctggagaccgtccagagactgctccccgtgctgtgccaagctcacggactcacacctgagcaggtcgtggcaatcgcctctaacattggagggaaacaggccctggaaactgtacagcggctgctccccgtgctgtgccaagcacacggactcactccacagcaggtcgtggccattgcaagtcatgacggaggcaagcaggccctggaaacagtgcagcgcctgctccctgtgctgtgccaggctcatggtctgactcctcagcaggtggtggccatcgcttccaacaatggagggaagcaggccctggagaccgtacagagactgctccccgtgctgtgccaagcgcacggtctgacccctcagcaggtcgtcgcaatcgccagcaatggcgggggcaagcaggctctcgaaaccgtccagcggctcctcccagtcctctgtcaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaacgatgtggtgctggagaaaagcgacatcgaaaaattcaagaaccagctgaggaccgagctgacaaatattgatcactcctacctgaagggaatcgacattgcctccaagaaaaagacctctaacgtggagaatacagagtttgaagctatctctactaagattttcaccgatgaactgggcttcagcgggaaacatctgggcggaagcaataagccagatggcctgctgtgggacgatgactgcgccatcattctggacagtaaggcttacagcgaggggttccccctgacagcctcccacactgacgctatgggcaggtatctgagacagtttactgagcggaaagaggaaatcaagcccacctggtgggatattgcccctgaacatctggacaacacctacttcgcttatgtgagcggctccttttctggaaattataaagagcagctgcagaagttccgccaggatacaaaccacctggggggcgccctggaatttgtgaagctgctgctgctggctaacaattacaaaactcagaagatgtccaaaaaggaggtgaaaaagtctatcctggactataacattagttacgaggaatatgcccccctgctggctgagatcgaatgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccggattc

SEQ ID NO: 35 - DNA, 7654 bp, synthetic construct
  misc_feature 1..7654: note = pCAG-ArtTal1-FokI
  source 1..7654: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttactccagaacaggtcgtcgcaatcgcaagtaacatcggcggaaaacaggccctcgaaaccgtccagagactcctccccgtgctgtgccaggcccacggactgaccccacagcaggtggtcgccatcgctagcaacggcggagggaagcaggctctggagaccgtgcagaggctgctccccgtcctgtgccaggcacatgggctcacacctcagcaggtggtcgcaattgcctccaatggtggcggaaaacaggccctggaaactgtgcagagactgctccccgtgctgtgccaggctcacggtctcacaccccagcaggtggtcgctatcgcatctcatgacgggggcaagcaggcactggagacagtgcagcggctgctccctgtcctgtgccaggcccacggactcactcctcagcaggtcgtcgccattgctagtaacggcggagggaaacaggctctggaaaccgtgcagcgcctgctccccgtgctgtgccaagcccacggcctgaccccccagcaggtggtcgcaatcgcctcaaacaatggtggcaagcaggccctggagactgtgcagcgactgctcccagtgctgtgccaggcccatggactcacaccacagcaggtcgtcgctattgcaagcaacaatggagggaaacaggcactggaaacagtccagaggctgctccccgtgctgtgccaagcgcatggactcactccccagcaggtcgtcgccatcgcttccaataacggcggcaagcaggccctggagaccgtccagagactgctccccgtgctgtgccaagctcacggactcacacctgagcaggtcgtggcaatcgcctctaacattggagggaaacaggccctggaaactgtacagcggctgctccccgtgctgtgccaagcacacggactcactccacagcaggtcgtggccattgcaagtcatgacggaggcaagcaggccctggaaacagtgcagcgcctgctccctgtgctgtgccaggctcatggtctgactcctcagcaggtggtggccatcgcttccaacaatggagggaagcaggccctggagaccgtacagagactgctccccgtgctgtgccaagcgcacggtctgacccctcagcaggtcgtcgcaatcgccagcaatggcgggggcaagcaggctctcgaaaccgtccagcggctcctcccagtcctctgtcaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaaccagctcgtgaaaagcgaactcgaagaaaagaaaagtgaactgcggcacaaactgaaatacgtcccacatgaatacattgagctgatcgagattgctaggaactccacccaggacagaatcctcgagatgaaagtgatggaattctttatgaaagtctacgggtatcggggcaagcacctgggcggatctcgcaaaccagatggggcaatctacactgtgggtagtcccatcgactatggcgtgattgtcgataccaaggcctacagtgggggttataatctgcccattggacaggctgacgagatgcagcgatacgtggaggaaaaccagacaagaaataagcatatcaaccccaatgagtggtggaaagtgtatcctagctccgtcactgaattcaagtttctcttcgtgtcaggccactttaagggaaactacaaagcacagctgaccaggctcaatcatattacaaactgcaatggcgccgtgctgagcgtcgaggaactgctcatcggcggagagatgatcaaggccggcacactcaccctggaggaggtccgccgaaaattcaataacggggaaatcaacttctgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccggattc

SEQ ID NO: 36 - protein, 947 aa, synthetic construct
  REGION 1..947: note = ArtTal1-Alw
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPEQVVAIASNIGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNKETNILLVEQLEETLNRNRILFEKNSSIAQAPIGEIKNYRYHLEELLFENNEKKFAENQKNEWDEILAYMDLLISPKPISIEIADKEISIPSGERPAYFEWVLWRAFLALNHLIIEPQQCRRFKVDQDFKPIHNAPGGGADVIFEYENFKILGEVTLTSNSRQEAAEGEPVRRHIAVETVNTPDKDVYGLFLALTIDTNTAETFRHGAWYHQEELMDVKILPLTLESFKKYLESLRKKNQVETGIFDLKKMMDESLKLRETLTAPQWKNEITNKFARPI

SEQ ID NO: 37 - protein, 869 aa, synthetic construct
  REGION 1..869: note = ArtTal1-CLEDORF
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPEQVVAIASNIGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNKLAKSSQSETKEKLREKLRNLPHEYLSLVDLAYDSKQNRLFEMKVIELLTEECGFQGLHLGGSRRPDGVLYTAGLTDNYGIILDTKAYSSGYSLPIAQADEMERYVRENQTRDELVNPNQWWENFENGLGTFYFLFVAGHFNGNVQAQLERISRNTGVLGAAASISQLLLLADAIRGGRMDRERLRHLMFQNEEFLLEQEL

SEQ ID NO: 38 - protein, 867 aa, synthetic construct
  REGION 1..867: note = ArtTal1-Clo051
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPEQVVAIASNIGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 39 - protein, 918 aa, synthetic construct
  REGION 1..918: note = ArtTal1-Mly
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPEQVVAIASNIGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNINSKIKQLDDSINVESLKIDDAKDLLNDLEIQRKAKTIEDTVNHLKLRSDIEDILDVFAKIKKRDVPDVPLFLEWNIWRAFAALNHTQAIEGNFIVDLDGMPLNTAPGKKPDIEINYGSFSCIVEVTMSSGETQFNMEGSSVPRHYGDLVRKVDHDAYCIFIAPKVAPGTKAHFFNLNRLSTKHYGGKTKIIPMSLDDFICFLQVGITHNFQDINKLKNWLDNLINFNLESEDEEIWFEEIISKISTWAI

SEQ ID NO: 40 - protein, 881 aa, synthetic construct
  REGION 1..881: note = ArtTal1-Pept071
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPEQVVAIASNIGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNKISKTNVLELKDKVRDKLKYVDHRYLALIDLAYDGTANRDFEIQTIDLLINELKFKGVRLGESRKPDGIISYNINGVIIDNKAYSTGYNLPINQADEMIRYIEENQTRDEKINSNKWWESFDDKVKDFNYLFVSSFFKGNFKNNLKHIANRTGVSGGAINVENLLYFAEELKAGRLSYVDSFKMYDNDEIYVGDFSDYSYVKFAAEEEGEYLT

SEQ ID NO: 41 - protein, 831 aa, synthetic construct
  REGION 1..831: note = ArtTal1-Sbf
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPEQVVAIASNIGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNISVDLPGGEEFLLSPAGQNPLLKKMVEEFVPRFAPRSTVLYLGDTRGKHSLFEREIFEEVLGLTFDPHGRMPDLILHDEVRGWLFLMEAVKSKGPFDEERHRSLQELFVTPSAGLIFVNCFENRESMRQWLPELAWETEAWVAEDPDHLIHLNGSRFLGPYER

SEQ ID NO: 42 - protein, 831 aa, synthetic construct
  REGION 1..831: note = ArtTal1-SdaI
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPEQVVAIASNIGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNISVDLADGDEFLLSPAGQNPLLKKMVEEFMPRFAPGAKVLYIGDWRGKHTRFEKRIFEETLGLTFDPHGRMPDLVLHDKVRKWLFLMEAVKSKGPFDEERHRTLRELFATPVAGLVFVNCFENREAMRQWLPELAWETEAWVADDPDHLIHLNGSRFLGPYER

SEQ ID NO: 43 - protein, 876 aa, synthetic construct
  REGION 1..876: note = ArtTal1-StsI
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPEQVVAIASNIGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNDVVLEKSDIEKFKNQLRTELTNIDHSYLKGIDIASKKKTSNVENTEFEAISTKIFTDELGFSGKHLGGSNKPDGLLWDDDCAIILDSKAYSEGFPLTASHTDAMGRYLRQFTERKEEIKPTWWDIAPEHLDNTYFAYVSGSFSGNYKEQLQKFRQDTNHLGGALEFVKLLLLANNYKTQKMSKKEVKKSILDYNISYEEYAPLLAEIE

SEQ ID NO: 44 - protein, 864 aa, synthetic construct
  REGION 1..864: note = ArtTal1-FokI
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPEQVVAIASNIGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 45 - DNA, 7374 bp, synthetic construct
  misc_feature 1..7374: note = ArtTal1-Reporter
  source 1..7374: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgacgacactgcagagacctacttcactaacaaccggtatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctataaaaccggtacagttcggccaccatggtcgtattctgggacgttttcacactcttctaacgtcccagaatactcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcctcgactctaggcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagcttgcatgcctgcaggt

SEQ ID NO: 46 - DNA, 7374 bp, synthetic construct
  misc_feature 1..7374: note = TalRab1-Reporter
  source 1..7374: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgacgacactgcagagacctacttcactaacaaccggtatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctataaaaccggtacagttcggccaccatggtcgtgtgcaccaaaacttttcacactcttctaagttttggtgcacacgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcctcgactctaggcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagcttgcatgcctgcaggt

SEQ ID NO: 47 - DNA, 7377 bp, synthetic construct
  misc_feature 1..7377: note = TalRab2-Reporter
  source 1..7377: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgacgacactgcagagacctacttcactaacaaccggtatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctataaaaccggtacagttcggccaccatggtcgatggtggcccggtagttttcacactcttctcactaccgggccaccacgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcctcgactctaggcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagcttgcatgcctgcaggt

SEQ ID NO: 48 - DNA, 7383 bp, synthetic construct
  misc_feature 1..7383: note = ArtTal1/TalRab2-Reporter
  source 1..7383: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggacactgcagagacctacttcactaacaaccggtatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctataaaaccggtacagttcggccaccatggtcgtattctgggacgtttttcacactcttctaaactaccgggccaccacgggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcctcgactctaggcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagcttgcatgcctgcaggt

SEQ ID NO: 49 - DNA, 5566 bp, synthetic construct
  misc_feature 1..5566: note = pCMV-hLuc
  source 1..5566: mol_type = other DNA
ggtaccgagctcttacgcgtgctagcccgggctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 50 - DNA, 2961 bp, synthetic construct
  misc_feature 1..2961: note = pBS
  source 1..2961: mol_type = other DNA
gtaaaacgacggccagtgagcgcgcgtaatacgactcactatagggcgaattggagctccaccgcggtggcggccgctctagaactagtggatcccccgggctgcaggaattcgatatcaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgtt

SEQ ID NO: 51 - DNA, 7164 bp, synthetic construct
  misc_feature 1..7164: note = pCMVbeta
  source 1..7164: mol_type = other DNA
gaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcaattcccggggatcgaaagagcctgctaaagcaaaaaagaagtcaccatgtcgtttactttgaccaacaagaacgtgattttcgttgccggtctgggaggcattggtctggacaccagcaaggagctgctcaagcgcgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcctcgactctaggcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 52 - DNA, 7867 bp, synthetic construct
  misc_feature 1..7867: note = pCAG-TalRab2-Clo051
  source 1..7867: mol_type = other DNA
ggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatctgacaccccagcaggtggtggccattgctagcaacaatgggggcaagcaggctctggagacagtgcagcgcctgctgcctgtgctgtgccaggctcacggactgactccacagcaggtggtggccatcgcttccaacaatggagggaaacaggctctggaaacagtgcagaggctgctgcccgtgctgtgccaggctcatggactgacacctcagcaggtcgtcgccattgcttctaacggcggagggaagcaggctctggagactgtgcagagactgctgccagtgctgtgccaggcccatggactgacccctcagcaggtcgtggctatcgctagtaacaatggcggaaaacaggctctggaaactgtgcagcggctgctccccgtgctgtgccaggcccacggcctcactccacagcaggtcgtcgctatcgcctctaataacgggggcaagcaggctctggagacagtacagcgcctgttacccgtgctgtgccaggcacacggcctcacacctcagcaggtcgtggcaatcgcttcccatgacggagggaaacaggctctggaaacggtccagaggctgctccccgtgctgtgccaagctcacggcctcacccctcagcaggtggtcgctattgcttctcatgatggcggaaagcaggctctggagaccgtgcagagactgctccctgtgctgtgccaagcccacggcctgactccacagcaggtcgtggccatcgctagtcatgacgggggcaaacaggctctggaaacagtacagcggctgttacccgtgctgtgccaagcccatggcctcacacctcagcaagtcgtcgctatcgctagcaacaatggagggaagcaggctctggagacggtgcagcgcctgctcccagtgctgtgccaagctcatggcctcacccctcagcaagtcgtcgcaattgcttccaataacggcggaaaacaggctctggaaaccgtccagaggctgctgcccgtgctgtgccaagcacatggcttaactccacagcaagtggtggccattgcttctaatgggggcggaaagcaggccctggagacagtccagagactgttgcccgtgctgtgccaagcgcatggactgacacctgaacaggtcgtcgctatcgctagtaatattgggggcaaacaggccctggaaacagtgcagcggctgcttcccgtgctgtgccaggcgcatggactcacaccccagcaggtcgtcgcaatcgcctctaataacggagggaagcaggccctggaaaccgtgcagagactgttacctgtgctgtgccaggcacatggtctgacaccacagcaggtggtcgcaattgctagcaatggcggagggaagcaggccctggagactgtccagagactgctacccgtgctgtgccaagcgcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaacgaaggcatcaaaagcaacatctccctcctgaaagacgaactccgggggcagattagccacattagtcacgaatacctctccctcatcgacctggctttcgatagcaagcagaacaggctctttgagatgaaagtgctggaactgctcgtcaatgagtacgggttcaagggtcgacacctcggcggatctaggaaaccagacggcatcgtgtatagtaccacactggaagacaactttgggatcattgtggataccaaggcatactctgagggttatagtctgcccatttcacaggccgacgagatggaacggtacgtgcgcgagaactcaaatagagatgaggaagtcaaccctaacaagtggtgggagaacttctctgaggaagtgaagaaatactacttcgtctttatcagcgggtccttcaagggtaaatttgaggaacagctcaggagactgagcatgactaccggcgtgaatggcagcgccgtcaacgtggtcaatctgctcctgggcgctgaaaagattcggagcggagagatgaccatcgaagagctggagagggcaatgtttaataatagcgagtttatcctgaaatactgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatga

SEQ ID NO: 53 - protein, 935 aa, synthetic construct
  REGION 1..935: note = TalRab2-Clo051
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASHDGGKQALETVQRLLPVLCQAHGLTPQQVVAIASHDGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 54 - DNA, 7867 bp, synthetic construct
  misc_feature 1..7867: note = pCAG-RabChtTal1-Clo051
  source 1..7867: mol_type = other DNA
ggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttacacccgaacaggtggtggccatcgctagtaacattgggggcaaacaggctctggaaacagtacagcggctgttacctgtgctgtgccaggctcatggcctcacacctcagcaggtcgtcgcaatcgcctccaatggcggagggaagcaggccctggaaacggtgcagagactgttaccagtgctgtgccaggcccatggcctaacaccccagcaggtggtggccatcgccagccacgacggcggcaagcaggccctggaaaccgtgcagaggctgctgcctgtgctgtgccaggctcatggcctgacacctgagcaggtcgtcgccatcgccagcaacatcggcggcaagcaggccctggaaaccgtgcagaggctgctgccagtgctgtgccaggcccatggcttaacacccgaacaggtggtggccatcgcttctaatattgggggcaagcaggccctggaaacagtccagagactgttgcctgtgctgtgccaggctcatggcttgacacctcagcaggtcgtcgctatcgcctctaataaggggggcaagcaggctctggagacagtacagcgcctgttaccagtgctgtgccaggcccacgggctcacaccccagcaggtggtggcaatcgcttcccatgacggagggaaacaggctctggaaacggtccagaggctgctccctgtgctgtgccaggctcacggtctaacaccccagcaggtggtggccattgctagcaacaatgggggcaagcaggctctggagacagtgcagcgcctgctgcctgtgctgtgccaggctcatggcctcacacctcagcaggtcgtcgccatcgccagccacgacggcggcaagcaggccctggaaaccgtgcagaggctgctgccagtgctgtgccaggcccatggcctaacaccccagcaggtggtggcaatcgcctccaatggcggagggaagcaggccctggaaacggtgcagagactgttacctgtgctgtgccaggctcatggcctgacacctgagcaggtcgtcgctatcgctagcaatatcggagggaagcaggctctggaaactgtccagcgcctgctcccagtgctgtgccaggcccatggcttaacaccccagcaggtggtggcaattgctagcaatggcggagggaagcaggccctggagactgtccagagactgctacctgtgctgtgccaggctcatggcttgacacctcagcaggtcgtcgctatcgcctctaataaggggggcaagcaggctctggagacagtacagcgcctgttaccagtgctgtgccaggcccacgggctcacaccccagcaggtggtggccatcgccagcaacggcggcggcaagcaggccctggaaaccgtgcagaggctgctgcctgtgctgtgccaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaacgaaggcatcaaaagcaacatctccctcctgaaagacgaactccgggggcagattagccacattagtcacgaatacctctccctcatcgacctggctttcgatagcaagcagaacaggctctttgagatgaaagtgctggaactgctcgtcaatgagtacgggttcaagggtcgacacctcggcggatctaggaaaccagacggcatcgtgtatagtaccacactggaagacaactttgggatcattgtggataccaaggcatactctgagggttatagtctgcccatttcacaggccgacgagatggaacggtacgtgcgcgagaactcaaatagagatgaggaagtcaaccctaacaagtggtgggagaacttctctgaggaagtgaagaaatactacttcgtctttatcagcgggtccttcaagggtaaatttgaggaacagctcaggagactgagcatgactaccggcgtgaatggcagcgccgtcaacgtggtcaatctgctcctgggcgctgaaaagattcggagcggagagatgaccatcgaagagctggagagggcaatgtttaataatagcgagtttatcctgaaatactgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatga

SEQ ID NO: 55 - protein, 935 aa, synthetic construct
  REGION 1..935: note = RabChtTal1-Clo051
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPEQVVAIASNIGGKQALETVQRLLPVLCQAHGLTPQQVVAIASNGGGKQALETVQRLLPVLCQAHGLTPQQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPQQVVAIASNKGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASHDGGKQALETVQRLLPVLCQAHGLTPQQVVAIASNGGGKQALETVQRLLPVLCQAHGLTPEQVVAIASNIGGKQALETVQRLLPVLCQAHGLTPQQVVAIASNGGGKQALETVQRLLPVLCQAHGLTPQQVVAIASNKGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 56 - DNA, 7867 bp, synthetic construct
  misc_feature 1..7867: note = pCAG-RabChtTal2-Clo051
  source 1..7867: mol_type = other DNA
ggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttacaccccagcaggtggtggccattgctagcaacaatgggggcaagcaggctctggagacagtgcagcgcctgctgcctgtgctgtgccaggctcatggcctcacacctcagcaggtcgtcgccattgcttctaacaatggagggaagcaggctctggagactgtgcagagactgctgccagtgctgtgccaggcccatggcctaacaccccagcaggtggtggccatcgccagccacgacggcggcaagcaggccctggaaaccgtgcagaggctgctgcctgtgctgtgccaggctcatggcctgacacctcagcaggtcgtcgccatcgccagccacgacggcggcaagcaggccctggaaaccgtgcagaggctgctgccagtgctgtgccaggcccatggcttaacaccccagcaggtggtggccatcgctagtcatgacgggggcaaacaggctctggaaacagtacagcggctgttacctgtgctgtgccaggctcatggcttgacacctcagcaggtcgtcgctatcgcctctaataaggggggcaagcaggctctggagacagtacagcgcctgttaccagtgctgtgccaggcccacgggctcacaccccagcaggtggtggcaattgcttccaataagggcggaaaacaggctctggaaaccgtccagaggctgctgcctgtgctgtgccaggctcacggtctaacaccccagcaggtggtggccatcgcttccaacggagggggcaaacaggctctggaaacagtgcagaggctgctgcctgtgctgtgccaggctcatggcctcacacctgagcaggtcgtcgccatcgccagcaacatcggcggcaagcaggccctggaaaccgtgcagaggctgctgccagtgctgtgccaggcccatggcctaacaccccagcaggtggtggcaattgcttccaataagggcggaaaacaggctctggaaaccgtccagaggctgctgcctgtgctgtgccaggctcatggcctgacacctcagcaggtcgtcgcaatcgcctccaatggcggagggaagcaggccctggaaacggtgcagagactgttaccagtgctgtgccaggcccatggcttaacaccccagcaggtggtggcaatcgcctctaataagggagggaagcaggccctggaaaccgtgcagagactgttacctgtgctgtgccaggctcatggcttgacacctcagcaggtcgtcgctatcgctagtcatgatggcggaaaacaggctctggaaactgtgcagcggctgctcccagtgctgtgccaggcccacgggctcacaccccagcaggtggtggccatcgccagcaacaagggcggcaagcaggccctggaaaccgtgcagaggctgctgcctgtgctgtgccaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaacgaaggcatcaaaagcaacatctccctcctgaaagacgaactccgggggcagattagccacattagtcacgaatacctctccctcatcgacctggctttcgatagcaagcagaacaggctctttgagatgaaagtgctggaactgctcgtcaatgagtacgggttcaagggtcgacacctcggcggatctaggaaaccagacggcatcgtgtatagtaccacactggaagacaactttgggatcattgtggataccaaggcatactctgagggttatagtctgcccatttcacaggccgacgagatggaacggtacgtgcgcgagaactcaaatagagatgaggaagtcaaccctaacaagtggtgggagaacttctctgaggaagtgaagaaatactacttcgtctttatcagcgggtccttcaagggtaaatttgaggaacagctcaggagactgagcatgactaccggcgtgaatggcagcgccgtcaacgtggtcaatctgctcctgggcgctgaaaagattcggagcggagagatgaccatcgaagagctggagagggcaatgtttaataatagcgagtttatcctgaaatactgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatga

SEQ ID NO: 57 - protein, 935 aa, synthetic construct
  REGION 1..935: note = RabChtTal2-Clo051
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPQQVVAIASHDGGKQALETVQRLLPVLCQAHGLTPQQVVAIASHDGGKQALETVQRLLPVLCQAHGLTPQQVVAIASHDGGKQALETVQRLLPVLCQAHGLTPQQVVAIASNKGGKQALETVQRLLPVLCQAHGLTPQQVVAIASNKGGKQALETVQRLLPVLCQAHGLTPQQVVAIASNGGGKQALETVQRLLPVLCQAHGLTPEQVVAIASNIGGKQALETVQRLLPVLCQAHGLTPQQVVAIASNKGGKQALETVQRLLPVLCQAHGLTPQQVVAIASNGGGKQALETVQRLLPVLCQAHGLTPQQVVAIASNKGGKQALETVQRLLPVLCQAHGLTPQQVVAIASHDGGKQALETVQRLLPVLCQAHGLTPQQVVAIASNKGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 58 - DNA, 6607 bp, synthetic construct
  misc_feature 1..6607: note = pRab38-chtTAL
  source 1..6607: mol_type = other DNA
caccgcattaccctgggcgttgaaaccgaagaagacctggatttgaaataggcgttttctttacatttctaaagtgggactcctcacttgtaaaaggaaaaataatgatacttttaagacttccaggatgactaaatggtgtgtatgagaagatttataaacatctgccgctacttacaatgataagaccacttgtgtgttgttcagcttggagaatttaggataggagtggaggctgaaagaaaagtaagcccttagcatttcctctcaggtggcctctactttaggtcattaacagttgaataggcgctaagagatagcattaccactttatagaagcccaggcaaaaggagattaaagggtttgcctaaattctttcaactctaagggccagagaagacctaagtctactgctttgctgtttctcaaggtctccccaactttacaacactgtgtgggtggcaacagggcttaatagcctcagaagacctgggtatttttcgacactcagttctctccccggcagaacgtggaaaacaaaatccacataagtttgtgtcatggacgggaggcgagagaaaaatctctgtgaaaggagtaaagcactgtgcaaataccagcttgacaggcagtagcactggggtcccgggtcctttagcttccagtcccaggagttgctcttgtctcctcccactctggagtccgcagagtaggaaggaggattaaacccgggggaggagttccgcaccagctccctatcctgcgccagcacgcctagcctaagcgcccacatagagctccggtctccgtcggtgcccagccccggctgtgcttcccagagcaagctccaggctccgcaagacccgcgggcctccaggatgcagacacctcacaaggagcacctgtacaagctgctggtgatcggcgacctggtagtgggcaagaccagcattattaaacggtacgtgcatcaaaatttctcctctcattatcgagccaccattggtgtggacttcgcgctgaaggtgctccactgggacccagagacggtggtgcgcttgcagctctgggacattgctggtgagcgatcagagcagcgcgcaacgggtgagggtggagtgagccagtgaggagttcgggggtgaaggttcggggagtggaaaatgacttttcagtcggttccagtcccgggacccttgagtgcaatcaagcaggagatccggatcgcctgggcgctccactcttggaaagtttggcttaatggcttggaaacctgatttcaaagaaatggaagtgttttcttttctttctttcctttttttttttttttttttttcttttgctgttgtttctgttggagtcgtccccactctacctgtaacttctagataacttcgctggctctcactggctgtgagaaagcgaaccactttctcctgggattcttgggtgcagagaaggctgtcgcctggactcacaaggagattgtagtcgcattcttgtttcattctagtccttttctggacacaggtagccgcgacttggcccagagtatctcacgtggctttcatccttcgtgtttagaggggaagcccctaggaaatttaagaaggagcaggattatcttaggaatttagtttctttcaaatctcactactatcatctccttgcttattggcctcttcagtcagaaaaatttgagatgctaaatttgtatacatctagaacgaactatctcttctcactccactcccctcttccccatctctcttccgtctccctccatccttggctatctcttcttcactttccatttcaaacaggagactgtgtatgttttttaggaaaacattaaaaaaaaaaccacaaaaacaaaaacaaaacggagacagggtcccgtcatgtaactctgctaacctatatcaagctgaccttgacctcatagagacccacttgcctctgcctccctagaggcaagggtcggggttatggtgatgttaatgtcgtttgctttaagattccttgatttgatcttggtgtattttttgagaaatctaaagtatgaaatcagagtttgactaacagcttctaccagctcctagccacaataaagactgaggcaggctatagttagtgctcaatactgggtcctacctggctgcttgtaacctgggcatgcctagcattctagatgctaactcaccaaagcagtagcattttaagctgcaaatggctaggcagcgacagctcaagaatcttcttgctttggagttttaaactccaatgagattttccatgatccctttcaaataaccctacttaatctctcttcatagcccacagtaccaagaagcctttgataagctctggattgaaaagaagcagttctttttcaaaagatgtgctcatttgaactagtgcatttccctggaaacactttgccaggacttgagatgggcactaagaaggaaaattcctcaaaggacatgtacagtcttgagatgcattcgcttctgtagccatgagcttgctggtcttgagataaggttagttggtgtagctaggttcatggtttggagtctttggcagttctagagaagcatgagctattagagacttggagattgcatcaagtagagccttttgagcttttcactgtgtacctgggccctctgtcgctgcacgttttagtgtctgaaatgtctttcagctgtagcagttttctcgggaccccagtttaaaatagcttactgtttaaaagatgtagctgtagctagcattattgaactagcataattatagtctaaatagcattatgtcttcagccttgttatatgttggtgagttttagtttcctcttctaaacgggaagaacagaaagatgtaatgattctgagcttccagagtgagacacctctagagagaaataccttcttctgaagactaccgtgtgattacagataaattctgatatctttgtttagcttttgatatctataaacagggagtgtattttatctctccaaatgagagaagaataaacaataatgcaaggtaaaggcaatagtgctacactctaggagttaccactctttgtacatttatttataaatactaagcaagaggaacatgccatacatacactgactaagtcctaacaagtggcagttcttatatcacacatttatcttgccctcaaatgccagtccagcatcagtttagtctcatgcatttggcagcataaggcagtttgagttccacacttgctctcagaagcaatttaactcccacacttgggaatcctttcctaagccacagtttcagaccaaagttttggtgaaggctataatcacagaagtctgcacaagtagggagtctgaaggatctgagctccattcagcagtcagagcggcatccaaccccaaggtaatgctcagctcactttgataacttcaagctcaaaggccctgaactgctgagttggaggttgaaagatgtttgggtaaaagcaaggtaattggcggataggatggttgtaacgtaattgtttcaagttgtattagagacctctgggttctaaggggatatgaaatccaacctccactctccactgagattcaagttaggttaagtatgcctttgagtaccctcaagtcacagcatgccactctccttttcttaactctaatatgtatctataaagaacgggtagtagtcaactgagtcgacggtatcgataagcttgatccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagct

SEQ ID NO: 59 - DNA, 202 bp, Mus musculus
  source 1..202: mol_type = other DNA
atgcagacacctcacaaggagcacctgtacaagctgctggtgatcggcgacctgggtgtgggcaagaccagcattatcaagcgctatgtgcaccaaaacttctcctcgcactaccgggccaccattggtgtggacttcgcgctgaaggtgctccactgggacccagagacggtggtgcgcttgcagctctgggacattgctg

SEQ ID NO: 60 - DNA, 8218 bp, synthetic construct
  misc_feature 1..8218: note = pRab38-chtTAL-neo
  source 1..8218: mol_type = other DNA
caccgcattaccctgggcgttgaaaccgaagaagacctggatttgaaataggcgttttctttacatttctaaagtgggactcctcacttgtaaaaggaaaaataatgatacttttaagacttccaggatgactaaatggtgtgtatgagaagatttataaacatctgccgctacttacaatgataagaccacttgtgtgttgttcagcttggagaatttaggataggagtggaggctgaaagaaaagtaagcccttagcatttcctctcaggtggcctctactttaggtcattaacagttgaataggcgctaagagatagcattaccactttatagaagcccaggcaaaaggagattaaagggtttgcctaaattctttcaactctaagggccagagaagacctaagtctactgctttgctgtttctcaaggtctccccaactttacaacactgtgtgggtggcaacagggcttaatagcctcagaagacctgggtatttttcgacactcagttctctccccggcagaacgtggaaaacaaaatccacataagtttgtgtcatggacgggaggcgagagaaaaatctctgtgaaaggagtaaagcactgtgcaaataccagcttgacaggcagtagcactggggtcccgggtcctttagcttccagtcccaggagttgctcttgtctcctcccactctggagtccgcagagtaggaaggaggattaaacccgggggaggagttccgcaccagctccctatcctgcgccagcacgcctagcctaagcgcccacatagagctccggtctccgtcggtgcccagccccggctgtgcttcccagagcaagctccaggctccgcaagacccgcgggcctccaggatgcagacacctcacaaggagcacctgtacaagctgctggtgatcggcgacctggtagtgggcaagaccagcattattaaacggtacgtgcatcaaaataccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggtttctcctctcattatcgagccaccattggtgtggacttcgcgctgaaggtgctccactgggacccagagacggtggtgcgcttgcagctctgggacattgctggtgagcgatcagagcagcgcgcaacgggtgagggtggagtgagccagtgaggagttcgggggtgaaggttcggggagtggaaaatgacttttcagtcggttccagtcccgggacccttgagtgcaatcaagcaggagatccggatcgcctgggcgctccactcttggaaagtttggcttaatggcttggaaacctgatttcaaagaaatggaagtgttttcttttctttctttcctttttttttttttttttttttcttttgctgttgtttctgttggagtcgtccccactctacctgtaacttctagataacttcgctggctctcactggctgtgagaaagcgaaccactttctcctgggattcttgggtgcagagaaggctgtcgcctggactcacaaggagattgtagtcgcattcttgtttcattctagtccttttctggacacaggtagccgcgacttggcccagagtatctcacgtggctttcatccttcgtgtttagaggggaagcccctaggaaatttaagaaggagcaggattatcttaggaatttagtttctttcaaatctcactactatcatctccttgcttattggcctcttcagtcagaaaaatttgagatgctaaatttgtatacatctagaacgaactatctcttctcactccactcccctcttccccatctctcttccgtctccctccatccttggctatctcttcttcactttccatttcaaacaggagactgtgtatgttttttaggaaaacattaaaaaaaaaaccacaaaaacaaaaacaaaacggagacagggtcccgtcatgtaactctgctaacctatatcaagctgaccttgacctcatagagacccacttgcctctgcctccctagaggcaagggtcggggttatggtgatgttaatgtcgtttgctttaagattccttgatttgatcttggtgtattttttgagaaatctaaagtatgaaatcagagtttgactaacagcttctaccagctcctagccacaataaagactgaggcaggctatagttagtgctcaatactgggtcctacctggctgcttgtaacctgggcatgcctagcattctagatgctaactcaccaaagcagtagcattttaagctgcaaatggctaggcagcgacagctcaagaatcttcttgctttggagttttaaactccaatgagattttccatgatccctttcaaataaccctacttaatctctcttcatagcccacagtaccaagaagcctttgataagctctggattgaaaagaagcagttctttttcaaaagatgtgctcatttgaactagtgcatttccctggaaacactttgccaggacttgagatgggcactaagaaggaaaattcctcaaaggacatgtacagtcttgagatgcattcgcttctgtagccatgagcttgctggtcttgagataaggttagttggtgtagctaggttcatggtttggagtctttggcagttctagagaagcatgagctattagagacttggagattgcatcaagtagagccttttgagcttttcactgtgtacctgggccctctgtcgctgcacgttttagtgtctgaaatgtctttcagctgtagcagttttctcgggaccccagtttaaaatagcttactgtttaaaagatgtagctgtagctagcattattgaactagcataattatagtctaaatagcattatgtcttcagccttgttatatgttggtgagttttagtttcctcttctaaacgggaagaacagaaagatgtaatgattctgagcttccagagtgagacacctctagagagaaataccttcttctgaagactaccgtgtgattacagataaattctgatatctttgtttagcttttgatatctataaacagggagtgtattttatctctccaaatgagagaagaataaacaataatgcaaggtaaaggcaatagtgctacactctaggagttaccactctttgtacatttatttataaatactaagcaagaggaacatgccatacatacactgactaagtcctaacaagtggcagttcttatatcacacatttatcttgccctcaaatgccagtccagcatcagtttagtctcatgcatttggcagcataaggcagtttgagttccacacttgctctcagaagcaatttaactcccacacttgggaatcctttcctaagccacagtttcagaccaaagttttggtgaaggctataatcacagaagtctgcacaagtagggagtctgaaggatctgagctccattcagcagtcagagcggcatccaaccccaaggtaatgctcagctcactttgataacttcaagctcaaaggccctgaactgctgagttggaggttgaaagatgtttgggtaaaagcaaggtaattggcggataggatggttgtaacgtaattgtttcaagttgtattagagacctctgggttctaaggggatatgaaatccaacctccactctccactgagattcaagttaggttaagtatgcctttgagtaccctcaagtcacagcatgccactctccttttcttaactctaatatgtatctataaagaacgggtagtagtcaactgagtcgacggtatcgataagcttgatccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagct

SEQ ID NO: 61 - DNA, 9989 bp, synthetic construct
  misc_feature 1..9989: note = pCMV-Rab-Reporter (hygro)
  source 1..9989: mol_type = other DNA
gaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggacactgcagagacctacttcactaacaaccggtatggtcgccagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctataaaaccggtacagttcggccaccatggtcgtatcaagcgctatgtgcaccaaaacttctcctcgcactaccgggccaccattggtcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaggggatcccccgggctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgagacggaaagaaccagctggggctcgatcctctagagtcgacgtttgatctgatatcatcgatgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccgatccagccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtcgagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatcaaacatgcatgaagttcctattccgaagttcctattctctagaaagtataggaacttcataaaacctgcaggcatgcaagcgatcgcggccggccaaggcccgcggggccactagttctagagcggccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 62 - DNA, 12 bp, synthetic construct
  misc_feature 1..12: note = DNA target sequence
  source 1..12: mol_type = other DNA
attctgggacgt

SEQ ID NO: 63 - DNA, 14 bp, Mus musculus
  source 1..14: mol_type = other DNA
ggtggcccggtagt

SEQ ID NO: 64 - protein, 34 aa, Xanthomonas campestris
LTPEQVVAIASNIGGKQALETVQRLLPVLCQAHG

SEQ ID NO: 65 - protein, 34 aa, Xanthomonas campestris
LTPQQVVAIASHDGGKQALETVQRLLPVLCQAHG

SEQ ID NO: 66 - protein, 34 aa, Xanthomonas campestris
LTPQQVVAIASNGGGKQALETVQRLLPVLCQAHG

SEQ ID NO: 67 - protein, 34 aa, Xanthomonas campestris
LTPQQVVAIASNNGGKQALETVQALLPVLCQAHG

SEQ ID NO: 68 - DNA, 12 bp, synthetic construct
  misc_feature 1..12: note = DNA target sequence
  source 1..12: mol_type = other DNA
attctgggacgt

SEQ ID NO: 69 - DNA, 12 bp, synthetic construct
  misc_feature 1..12: note = DNA target sequence
  source 1..12: mol_type = other DNA
acgtcccagaat

SEQ ID NO: 70 - DNA, 12 bp, synthetic construct
  misc_feature 1..12: note = DNA target sequence
  source 1..12: mol_type = other DNA
taagaccctgca

SEQ ID NO: 71 - DNA, 12 bp, synthetic construct
  misc_feature 1..12: note = DNA target sequence
  source 1..12: mol_type = other DNA
tgcagggtctta

SEQ ID NO: 72 - DNA, 13 bp, synthetic construct
  misc_feature 1..13: note = DNA target sequence
  source 1..13: mol_type = other DNA
gtgcaccaaaact

SEQ ID NO: 73 - DNA, 13 bp, synthetic construct
  misc_feature 1..13: note = DNA target sequence
  source 1..13: mol_type = other DNA
agttttggtgcac

SEQ ID NO: 74 - DNA, 13 bp, synthetic construct
  misc_feature 1..13: note = DNA target sequence
  source 1..13: mol_type = other DNA
cacgtggttttga

SEQ ID NO: 75 - DNA, 13 bp, synthetic construct
  misc_feature 1..13: note = DNA target sequence
  source 1..13: mol_type = other DNA
tcaaaaccacgtg

SEQ ID NO: 76 - DNA, 14 bp, synthetic construct
  misc_feature 1..14: note = DNA target sequence
  source 1..14: mol_type = other DNA
actaccgggccacc

SEQ ID NO: 77 - DNA, 14 bp, synthetic construct
  misc_feature 1..14: note = DNA target sequence
  source 1..14: mol_type = other DNA
ccaccgggccatca

SEQ ID NO: 78 - DNA, 14 bp, synthetic construct
  misc_feature 1..14: note = DNA target sequence
  source 1..14: mol_type = other DNA
tgatggcccggtgg

SEQ ID NO: 79 - DNA, 12 bp, synthetic construct
  misc_feature 1..12: note = DNA target sequence
  source 1..12: mol_type = other DNA
attctgggacgt

SEQ ID NO: 80 - DNA, 14 bp, synthetic construct
  misc_feature 1..14: note = DNA target sequence
  source 1..14: mol_type = other DNA
actaccgggccacc

SEQ ID NO: 81 - DNA, 12 bp, synthetic construct
  misc_feature 1..12: note = DNA target sequence
  source 1..12: mol_type = other DNA
taagaccctgca

SEQ ID NO: 82 - DNA, 14 bp, synthetic construct
  misc_feature 1..14: note = DNA target sequence
  source 1..14: mol_type = other DNA
tgatggcccggtgg

SEQ ID NO: 83 - DNA, 69 bp, synthetic construct
  misc_feature 1..69: note = Rab38 Exon 1 partial sequence
  source 1..69: mol_type = other DNA
tgggtgtgggcaagaccagcattatcaagcgctatgtgcaccaaaacttctcctcgcactaccgggcca

SEQ ID NO: 84 - DNA, 69 bp, synthetic construct
  misc_feature 1..69: note = Rab38 Exon 1 partial sequence
  source 1..69: mol_type = other DNA
acccacacccgttctggtcgtaatagttcgcgatacacgtggttttgaagaggagcgtgatggcccggt

SEQ ID NO: 85 - DNA, 100 bp, synthetic construct
  misc_feature 1..100: note = Rab38 Exon 1 partial sequence
  source 1..100: mol_type = other DNA
tgatcggcgacctggtagtgggcaagaccagcattattaaacggtacgtgcatcaaaatttctcctctcattatcgagccaccattggtgtggacttcgc

SEQ ID NO: 86 - DNA, 100 bp, synthetic construct
  misc_feature 1..100: note = Rab38 Exon 1 partial sequence
  source 1..100: mol_type = other DNA
actagccgctggaccatcacccgttctggtcgtaataatttgccatgcacgtagttttaaagaggagagtaatagctcggtggtaaccacacctgaagcg

SEQ ID NO: 87 - protein, 24 aa, synthetic construct
  REGION 1..24: note = Rab38 Exon 1 partial sequence
DLVVGKTSIIKRYVHQNFSSHYRA

(19) United States
(12) Patent Application Publication
(10) Pub. No.: US 2025/0388934 A1
(43) Pub. Date: Dec. 25, 2025

(54) PROTEIN HAVING NUCLEASE ACTIVITY, FUSION PROTEINS AND USES THEREOF

(71) Applicant: Helmholtz Zentrum München Deutsches Forschungszentrum für Gesundheit und Umwelt (GmbH), Neuherberg (DE)

(72) Inventor: Ralf KÜHN, Berlin (DE)

(21) Appl. No.: 19/028,088

(22) Filed: Jan. 17, 2025

Related U.S. Application Data
(63) Continuation of application No. 17/481,484, filed on Sep. 22, 2021, now abandoned.
Application No. 17/481,484 is a continuation of application No. 15/960,364, filed on Apr. 23, 2018, now Pat. No. 11,149,289.
Application No. 15/960,364 is a continuation of application No. 15/198,967, filed on Jun. 30, 2016, now abandoned.
Application No. 15/198,967 is a division of application No. 14/124,117, filed on Mar. 18, 2014, now Pat. No. 9,410,134.

(30) Foreign Application Priority Data
Jun. 7, 2011 (EP) 11004635.6

(51) Int. Cl. C12N 15/90; A01K 67/0275; C07K 14/195; C12N 5/075; C12N 9/16; C12N 9/22; C12N 15/62; C12N 15/85

(52) U.S. Cl. CPC C12N 15/907; A01K 67/0275; C07K 14/195; C12N 5/0609; C12N 9/16; C12N 9/22; C12N 15/62; C12N 15/8509; C12Y 301/21004; A01K 2207/05; A01K 2217/07; A01K 2217/075; A01K 2227/105; A01K 2267/03; C07K 2319/80; C12N 2800/80; C12N 2999/007

(57) ABSTRACT

The present invention relates to a nucleic acid molecule encoding (I) a polypeptide having the activity of an endonuclease, which is (a) a nucleic acid molecule encoding a polypeptide comprising or consisting of the amino acid sequence of SEQ ID NO: 1; (b) a nucleic acid molecule comprising or consisting of the nucleotide sequence of SEQ ID NO: 2; (c) a nucleic acid molecule encoding an endonuclease, the amino acid sequence of which is at least 70% identical to the amino acid sequence of SEQ ID NO: 1; (d) a nucleic acid molecule comprising or consisting of a nucleotide sequence which is at least 50% identical to the nucleotide sequence of SEQ ID NO: 2; (e) a nucleic acid molecule which is degenerate with respect to the nucleic acid molecule of (d); or (f) a nucleic acid molecule corresponding to the nucleic acid molecule of any one of (a) to (e) wherein T is replaced by U; (II) a fragment of the polypeptide of (I) having the activity of an endonuclease. Also, the present invention relates to a vector comprising the nucleic acid molecule and a protein encoded by said nucleic acid molecule. Further, the invention relates to a method of modifying the genome of a eukaryotic cell and a method of producing a non-human vertebrate or mammal.

### CROSS-REFERENCE TO RELATED APPLICATIONS

This application is a continuation of U.S. patent application Ser. No. 17/481,484, filed on Sep. 22, 2021, which is a continuation of U.S. patent application Ser. No. 15/960,364, filed on Apr. 23, 2018 (now U.S. Pat. No. 11,149,289, issued on Oct. 19, 2021), which is a continuation of U.S. patent application Ser. No. 15/198,967, filed on Jun. 30, 2016 (abandoned), which is a divisional of U.S. patent application Ser. No. 14/124,117, filed on Mar. 18, 2014 (now U.S. Pat. No. 9,410,134, issued on Jul. 20, 2016), which is the U.S. national stage of International Patent Application No. PCT/EP2012/060711, filed on Jun. 6, 2012, which claims priority to European Patent Application No. 11004635.6, filed on Jun. 7, 2011, the contents of which are incorporated by reference in their entirety.

### DESCRIPTION OF THE TEXT FILE SUBMITTED ELECTRONICALLY

The contents of the text file named “POTH-010_C03US_SeqList.xml”, which was created on Jan. 17, 2025 and is 302,400 bytes in size, and filed concurrently herewith, is hereby incorporated by reference in its entirety in this application.

### FIELD OF THE INVENTION

The present invention relates to a nucleic acid molecule encoding (I) a polypeptide having the activity of an endonuclease, which is (a) a nucleic acid molecule encoding a polypeptide comprising or consisting of the amino acid sequence of SEQ ID NO: 1; (b) a nucleic acid molecule comprising or consisting of the nucleotide sequence of SEQ ID NO: 2; (c) a nucleic acid molecule encoding an endonuclease, the amino acid sequence of which is at least 70% identical to the amino acid sequence of SEQ ID NO: 1; (d) a nucleic acid molecule comprising or consisting of a nucleotide sequence which is at least 50% identical to the nucleotide sequence of SEQ ID NO: 2; (e) a nucleic acid molecule which is degenerate with respect to the nucleic acid molecule of (d); or (f) a nucleic acid molecule corresponding to the nucleic acid molecule of any one of (a) to (e) wherein T is replaced by U; (II) a fragment of the polypeptide of (I) having the activity of an endonuclease. Also, the present invention relates to a vector comprising the nucleic acid molecule and a protein encoded by said nucleic acid molecule. Further, the invention relates to a method of modifying the genome of a eukaryotic cell and a method of producing a non-human vertebrate or mammal.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Nucleases remain to be one of the most important tools of molecular biologists since their discovery in the late 1960s. Nucleases are enzymes capable of cleaving the phosphodiester bonds between the nucleotide subunits of nucleic acids. Enzymes catalyzing DNA and RNA cleavage are integral parts of major DNA metabolic processes such as DNA replication, DNA recombination, DNA repair, site-specific recombination and RNA splicing. In addition, nuclease activities are essential in RNA processing, maturation, RNA interference and are components of microbial defense mechanisms.

RNA and DNA present only two types of phosphodiester bonds for cleavage, 5′- or 3′- of a scissile phosphate and the fundamental chemistry is bimolecular nucleophilic substitution. Nonetheless, structures and catalytic mechanisms of RNA and DNA nucleases are greatly varied and complex. Nucleases may be endo- or exonucleases, DNA or RNA specific, topoisomerases, recombinases, ribozymes, or RNA splicing enzymes. Their reaction can be divided into the three stages of nucleophilic attack, the formation of a negatively charged penta-covalent intermediate and the breakage of the scissile bond. Nucleases utilize a variety of nucleophiles to cleave a scissile phosphate bond. The most common nucleophiles are water molecules deprotonated by a general base for direct hydrolysis. For DNA cleavage, the side chains of Ser, Tyr and His serve as nucleophiles to form a covalent DNA phosphoryl-protein intermediate, which is subsequently resolved either by phosphoryl transfer reaction back to DNA during recombination and topoisomerization or by hydrolysis in two-step cleavage reactions. To enable the controlled degradation or processing of cellular DNA or RNA, nuclease activities are strictly regulated by stringent substrate specificity, confined localization, or by potent inhibitors.

For convenience nucleases can be classified according to their catalytic mechanism into three major classes based on their metal-ion dependence (Yang, W. (2011). Q. Rev. Biophys. 44(1): 1-93). These classes of two-metal-ion-dependent, one-metal-ion-dependent and metal-independent nucleases are further divided into families or superfamilies according to sequence and structure conservation and functional diversity.

### Restriction Endonucleases

Various families of restriction endonucleases are found among all three catalytic classes. The type I, Ill and IV restriction enzymes are multisubunit and complex molecular machines that combine multiple activities including restriction, methylation and DNA translocation, require additional cofactors (AdoMet, ATP or GTP), bind more than one target site, and cleave outside the recognition sequence, often at a random distance. Type II restriction endonucleases are enzymes that recognize short DNA sequences (usually 4-8-bp long) and cleave the target in both strands at, or in close proximity to the recognition site. Orthodox type II restriction enzymes are homodimeric, cleave within palindromic sequences, require Mg2⁺ ions and can act on single copies of their targets. Because of their remarkably high specificity in recognizing and cleaving their target sequences, they are of high interest as the most frequently used tools for recombinant DNA technology (Pingoud, A., M. Fuxreiter, et al. (2005). Cell Mol Life Sci 62(6): 685-707; Orlowski, J. and J. M. Bujnicki (2008). Nucleic Acids Res 36(11): 3552-69). In nature, type II REases (restriction endonucleases) are found in prokaryotic organisms, where they form restriction-modification systems with DNA methyltransferases of the same or very similar substrate specificity. DNA methyltransferases use S-adenosylmethionine (AdoMet) as a methyl group donor to modify specific bases in the target sequence, thereby rendering it resistant to cleavage by the restriction enzyme. While the Restriction-Modification system's own DNA is protected against self-degradation by the nuclease, any foreign DNA (e.g. from phages) that invades the host cell and lacks methylation, can be efficiently destroyed. In order to distinguish the components of restriction-modification systems the names of methylases and nucleases are preceded with ‘M’. and ‘R.’ prefixes (e.g. M.FokI and R.FokI).

Many commonly used type-II restriction endonucleases share the conserved motif PD-(D/E)XK. Said motif is generally found in proteins that interact with nucleic acid molecules such as DNA and is not limited to the presence in nucleases. The three catalytic residues are located close to each other on an uneven n-hairpin. The first D is located at the beginning of the first and shorter strand, and the E and K, separated by a hydrophobic residue x, are located in the middle of the second and longer strand. The first D is most conserved and coordinates both metal ions, whereas the second E can be replaced by Q, D, N, H or S, and the third K can be replaced E, Q, D, S, N or T. By varying dimeric interfaces and thus the relative positions of the two catalytic centers, dimeric endonucleases can cleave DNA to generate blunt ends or staggered ends with various 5′- or 3′-overhangs. The catalytic module invariably approaches DNA from the minor groove side, and the sequence-specific binding is conducted by a separate module/subdomain in the major groove. The first two carboxylates of the DEK motif coordinate the metal ions. The third, which usually is hydrogen bonded with both the nucleophilic water and the DNA-binding module in the major groove, couples DNA sequence recognition with the cleavage reaction. Members of this superfamily have a very diverse primary sequence and thus different structures surrounding the catalytic core. Database searches with restriction enzyme sequences typically reveal either no significant similarity to any protein, or very high similarity (>90% identity) to a few isoschizomers, and no similarity to other proteins. This strongly biased distribution of similarities and dissimilarities made comparative sequence analysis of all restriction enzymes difficult and raised a question whether the diversity of amino acid sequences of restriction endonucleases indicates polyphyletic evolution (convergence) or extreme divergence from a common ancestor.

While ˜70% of restriction endonucleases belong to the PD-(D/E)XK superfamily, other superfamily members can be monomeric or tetrameric and be involved in other processes such as DNA repair and homologous recombination. In addition to endonucleases, members in this superfamily can also be 5′- or 3′-exonucleases. The most comprehensive source of information on restriction enzymes is the REBASE database (rebase.neb.com) that lists several thousand functionally characterized enzymes and several thousand putative enzymes, inferred from sequence comparisons or genomic analyses. Therefore, a large disproportion exists between the number of known or predicted sequences and the small number of ˜50 experimentally characterized proteins with known three-dimensional structures. Presently, a large fraction of putative enzymes remains without any predictions or experimental data.

Type II REases are further subdivided into several types according to their recognition site symmetry, structural organization or cofactor requirement. Most of the restriction enzymes used for recombinant DNA work belong to type HP (P-palindromic). Type HA enzymes recognize asymmetric sequences, like Bpu101, a dimer of non-identical subunits, each of which is responsible for cleavage of one strand of the DNA. Type IIB enzymes cleave DNA at both sides of the recognition sequence, an example being BpII that cleaves the topstrand 8 nucleotides before and 13 nucleotides after the recognition sequence, while the bottom strand is cleaved 13 nucleotides before and 8 nucleotides after the recognition sequence. Type IIC enzymes have both cleavage and modification domains within one polypeptide. Type IIE enzymes need to interact with two copies of their recognition sequence for efficient cleavage, one copy being the target for cleavage, the other serving as an allosteric effector. Type IIE enzymes like Nael recognize palindromic nucleotide sequences in a manner similar to the type IIP enzymes and cleave DNA within the boundaries of their recognition sites; however, they possess a separate DNA binding domain to perform allosteric function. Type IIF enzymes are typically homotetrameric restriction endonucleases that also interact with two copies of their recognition site, but cleave both of them in a concerted manner. Type IIG enzymes, essentially a subgroup of Type IIC enzymes, have both cleavage and modification domains within one polypeptide. They are in general stimulated by AdoMet, but otherwise behave as typical Type II enzymes. Type IIH enzymes behave like type II enzymes, but their genetic organization resembles Type I Restriction-Modification systems. Type IIM enzymes recognize a specific methylated sequence and cleave the DNA at a fixed site. The best known representative is Dpnl which cleaves Gm6ATC, Gm6ATm4C and Gm6ATm5C, yet not GATC, GATm4C, GATm5C or hemimethylated sites. Many other restriction enzymes are more or less tolerant to methylation, but for Type IIM enzymes the methyl group is an essential recognition element. Orthodox Type IIP enzymes like EcoRI recognize symmetric nucleotide sequences and cleave within their recognition sites. They share both a common structural core comprising the five stranded mixed n-sheet flanked by α-helices. The DNA binding sites of Type IIP enzymes, however, are highly diverse and usually form a patch on the protein surface composed of amino acid residues located on the different structural elements (α-helices, β-strands, loops). Orthodox Type IIP enzymes interact with DNA as homodimers, and each subunit contributes to the recognition of half of the palindromic sequence. Type IIS enzymes cleave at least one strand of the target DNA outside of the recognition sequence. The best-known type IIS enzyme is FokI, which like many other type IIS enzymes interacts with two recognition sites before cleaving DNA. Type IIS enzymes are active as homodimers and are composed of two domains, one responsible for target recognition and the other for catalysis (also serving as the dimerization domain). This is apparent from the crystal structure and biochemical studies of FokI (Bitinaite, J., D. A. Wah, et al. (1998). Proc Natl Acad Sci USA 95(18): 10570-5; Wah, D. A., J. Bitinaite, et al. (1998). Proc Natl Acad Sci USA 95(18): 10564-9). Crystal structure analysis of FokI reveals that it is composed of a specific DNA binding module fused to the cleavage domain that possesses a conserved endonuclease catalytic core but cuts DNA in a nonspecific manner. Modular architecture is also characteristic for the type IIS enzyme BfiI, which is composed of two DNA binding domains fused to the dimeric catalytic core similar to the nonspecific nuclease belonging to the phospholipase D family. The presence of a separate nuclease domain has been also reported from the crystal structure of the Type IIP enzyme SdaI (Tamulaitiene, G., A. Jakubauskas, et al. (2006). Structure 14(9): 1389-400)

### Modified Restriction Enzymes and Chimaeric Nucleases as Tools for Qenome Editing

Nucleases that cleave nucleic acid molecules at specific sites rather than randomly are of increasing importance in emerging technologies such as, e.g., in genetic engineering and gene targeting. Gene targeting is a process in which a DNA molecule introduced into a cell replaces the corresponding chromosomal segment by homologous recombination, and thus presents a precise way to manipulate the genome (Capecchi, M. R. (2005). Nat Rev Genet 6(6): 507-12). In the past, the application of gene targeting to mammalian cells has been limited by its low efficiency. Experiments in model systems have demonstrated that the frequency of homologous recombination of a gene targeting vector is strongly increased if a double-strand break is induced within its chromosomal target sequence. Using the yeast homing endonuclease I-SceI, that cuts DNA at an 18 base pair-long recognition site, it was initially shown that homologous recombination and gene targeting are stimulated over 1000-fold in mammalian cells when a recognition site is inserted into a target gene and I-SceI is expressed in these cells (Rouet, P., Smih, F., Jasin, M.; Mol Cell Biol 1994; 14: 8096-8106; Rouet, P., Smih, F. Jasin, M; Proc Natl Acad Sci USA 1994; 91: 6064-6068). In the absence of a gene targeting vector for homology directed repair, the cells frequently close the double-strand break by non-homologous end-joining (NHEJ). Since this mechanism is error-prone it frequently leads to the deletion or insertion of multiple nucleotides at the cleavage site. If the cleavage site is located within the coding region of a gene it is thereby possible to identify and select mutants that exhibit reading frameshift mutations from a mutagenised population and that represent non-functional knockout alleles of the targeted gene.

Therefore, sequence specific nucleases represent an important tool for biotechnology to modify the genome of model organisms or cell lines. In order to construct nucleases that specifically recognise new target sequences within genes, two approaches have been pursued that rely on the modification of natural homing endonucleases or on the fusion of a natural or engineered DNA binding domain to a nuclease domain. Such modified restriction enzymes or chimaeric nucleases can target large DNA sites (up to 36 bp) and can be engineered to bind to desired DNA sequences.

Homing endonucleases, such as I-SceI of yeast, are natural genetic elements that catalyze their own duplication into recipient alleles by creating site-specific DSBs that initiate their own genetic transfer by homologous recombination. A key feature of these enzymes is that they create double-strand breaks at recognition sites that are 14- to 40-bp long. The major limitation to the use of homing endonucleases in gene targeting is that each enzyme recognises exclusively its natural target sequence. By protein engineering it has been attempted to modify homing endonucleases in order to recognize new target sites. In this work, modifications could be made that alter the natural target site within some nucleotides, but it is yet not possible to design enzymes specific for entirely new target regions.

Due to the difficulty of manipulating the sequence recognition of homing enonucleases, zinc-finger nucleases (ZFN) are presently the most commonly used artificial nucleases for genetic engineering (Umov, F. D., E. J. Rebar, et al. Nat Rev Genet 11(9): 636-46). Zinc-finger nucleases were developed by fusing the nonsequence-specific cleavage domain of the FokI type IIS restriction endonuclease (Fn domain) to a new DNA binding domain. The advantage of zinc-finger nucleases is that the zinc-finger DNA binding domain can be modified to recognize novel target sequences, including those in endogenous genes. The protein modules known as zinc-fingers are found in the DNA-binding domain of the most abundant family of transcription factors in most eukaryotic genomes. Each finger is composed of 30 amino-acids, coordinates one Zn2+-ion using two cysteines and two histidine residues, and contacts primarily three basepairs of DNA. Two critical features of the structure are that each finger binds its 3-bp target site independently and that each nucleotide seemed to be contacted by a single amino acid side chain projecting from one end of the a-helix into the major groove of the DNA. Individual fingers have been designed to recognize many of the 64 different target triplets, but the greatest success has been in designing zinc fingers to recognize 5′-GNN-3′ triplets. Although zinc-finger recognition codes have been proposed, no code currently exists that consistently results in zinc-fingers with high affinity binding. Improving the specificity of zinc-finger binding, such as by increasing the number of fingers or by constructing multifinger proteins using two-finger units, remains an active area of research.

Using zinc-finger nucleases in the absence of a gene targeting vector for homology directed repair, knockout alleles were generated in mammalian cell lines and knockout zebra fish and rats were obtained upon the expression of ZFN mRNA in one cell embryos (Santiago Y, Chan E, Liu P Q, Orlando S, Zhang L, Umov F D, Holmes M C, Guschin D, Waite A, Miller J C, Rebar E J, Gregory P D, Klug A, Collingwood T N; Proc Natl Acad Sci USA 2008; 105:5809-5814; Doyon Y, McCammon J M, Miller J C, Faraji F, Ngo C, Katibah G E, Amora R, Hocking T D, Zhang L, Rebar E J, Gregory P D, Urnov F D, Amacher S L.; Nat Biotechnol 2008; 26:702-708; Geurts A M, Cost G J, Freyvert Y, Zeitler B, Miller J C, Choi V M, Jenkins S S, Wood A, Cui X, Meng X, Vincent A, Lam S, Michalkiewicz M, Schilling R, Foeckler J, Kalloway S, Weiler H, Menoret S, Anegon I, Davis G D, Zhang L, Rebar E J, Gregory P D, Urnov F D, Jacob H J, Buelow R.; Science 2009; 325:433). Furthermore, zinc-finger nucleases were used in the presence of exogeneous gene targeting vectors that contain homology regions to the target gene for homology driven repair of the double strand break through gene conversion. This methodology has been applied to gene engineering in mammalian cell lines and gene correction in primary human cells (Umov F D, Miller J C, Lee Y L, Beausejour C M, Rock J M, Augustus S, Jamieson A C, Porteus M H, Gregory P D, Holmes M C.; Nature 2005; 435:646-651; Porteus M H, Baltimore D. 2003. Science 300:763; Hockemeyer D, Soldner F, Beard C, Gao Q, Mitalipova M, DeKelver R C, Katibah G E, Amora R, Boydston E A, Zeitler B, Meng X, Miller J C, Zhang L, Rebar E J, Gregory P D, Urnov F D, Jaenisch R.; Nat Biotechnol 2009; 27:851-857).

Although the use of zinc-finger nucleases results in a higher frequency of homologous recombination, considerable efforts and time are required to design zinc-finger proteins that bind a new DNA target sequence at high efficiency and that act as sequence specific nuclease. In addition, it has been long ignored that the nature of the nuclease domain of zinc-finger and other chimaeric nucleases may represent an equally important success factor for the overall activity of the fusion protein. The reason for this neglection is based on the fact that up to date only a single nuclease domain has been found that retains nuclease activity within a separate protein folding domain and that can be combined with DNA binding domains, in order to generate a sequence specific nuclease fusion proteins. This nuclease domain is derived from the type IIS FokI restriction enzyme that has been characterised in detail and is known to act as an obligate dimer (Bitinaite, J., D. A. Wah, et al. (1998). Proc Natl Acad Sci USA 95(18): 10570-5; Wah, D. A., J. Bitinaite, et al. (1998). Proc Natl Acad Sci USA 95(18): 10564-9). In most other restriction enzymes DNA recognition and cleavage are combined into a single protein domain and cannot be separated. An exception is the SdaI enzyme that has been structurally characterised to posses a separate nuclease domain (Tamulaitiene, G., A. Jakubauskas, et al. (2006). Structure 14(9): 1389-400). In addition, it has not been possible to isolate mutants that loose DNA recognition but retain DNA cleavage activity.

Therefore, due to the lack other comparable functional nuclease domains, it was for a long time essentially unknown whether the enzymatic properties of the FokI Fn domain may constitute a limiting factor for the nuclease activity of Fn domain fusion proteins. For example, the intrinsic structure of the Fn domain may restrict its enzymatic processivity or the small dimerisation interface of two Fn domains may lead to a suboptimal interaction and a low cleavage rate of the DNA substrate.

By site-directed mutagenesis the FokI Fn domain has been engineered into the KK and EL variants that preferentially act as heterodimers (Miller, J. C., M. C. Holmes, et al. (2007). Nat Biotechnol 25(7): 778-85). The use of these variants provides the improved target sequence specificity of zinc-finger nucleases and reduces toxicity in mammalian cells since less genomic off-target sequences are recognised and processed. However, the overall nuclease activity of the KK and EL variants is at most comparable to that of the Fn wildtype domain.

Only very recently it has been found that the wildtype FokI Fn domain indeed exhibits only a suboptimal enzymatic nuclease activity that limits the use of zinc-finger nucleases for genome engineering. In a study of directed protein evolution the Fn domain has been randomly mutagenised and subjected to an E. coli based nuclease assay able to select mutants that exhibit increased enzymatic activity (Guo, J., T. Gaj, et al. (2010), J Mol Biol 400(1): 96-107). By this procedure it has been possible to isolate mutants that exhibit >10-fold higher nuclease activity as compared to the wildtype Fn domain. Upon coupling of these mutants to zinc-finger domains such fusion proteins showed a three to sixfold improved substrate processing in mammalian cells. However, it remains unknown at present whether the activity of the Fn domain can be further enhanced or whether the intrinsic protein architecture of the Fn domain may restrict any further improvements.

Besides zinc-finger DNA-binding domains fused to nuclease domains, very recently also TAL effector protein DNA-binding domains have been identified. As compared to zinc-finger motifs, TAL repeat elements within TAL effector proteins provide a new type of DNA binding domain that may be combined with a nuclease domain into sequence specific nucleases. A key feature of the TAL peptide elements is provided by their modulatory nature. Thereby, new sequence specific DNA-binding proteins can be generated through the combination of just four basic TAL elements that are each specific for the A, C, G or T nucleotide. Currently, only the nuclease domain of FokI is successfully used in fusion with TAL effector protein DNA-binding domains (Miller et al. (2010). Nat. Biotechnol. 29, 143-148).

In summary, there is an ongoing need for nucleases that can be used in various experimental settings including their fusion to other proteins and modification of the nuclease domain.

The technical problem underlying the present invention was to identify alternative and/or improved means and methods for cleaving nucleic acid molecules.

The solution to this technical problem is achieved by providing the embodiments characterized in the claims.

Accordingly, the present invention relates in a first embodiment to a nucleic acid molecule encoding (I) a polypeptide having the activity of an endonuclease, which is (a) a nucleic acid molecule encoding a polypeptide comprising or consisting of the amino acid sequence of SEQ ID NO: 1; (b) a nucleic acid molecule comprising or consisting of the nucleotide sequence of SEQ ID NO: 2; (c) a nucleic acid molecule encoding an endonuclease, the amino acid sequence of which is at least 70% identical to the amino acid sequence of SEQ ID NO: 1; (d) a nucleic acid molecule comprising or consisting of a nucleotide sequence which is at least 50% identical to the nucleotide sequence of SEQ ID NO: 2; (e) a nucleic acid molecule which is degenerate with respect to the nucleic acid molecule of (d); or (f) a nucleic acid molecule corresponding to the nucleic acid molecule of any one of (a) to (e) wherein T is replaced by U; (II) a fragment of the polypeptide of (I) having the activity of an endonuclease.

In accordance with the present invention the term “nucleic acid molecule” defines a linear molecular chain consisting of at least (for each) 2, 5, 10, 25, 50, 75, 100, 250, 500, such as at least 750, 1000, or at least 2500 or more nucleotides. The group of molecules designated herein as “nucleic acid molecules” also comprises complete genes. The term “nucleic acid molecule” is interchangeably used herein with the term “polynucleotide”.

The term “nucleic acid molecule” in accordance with the present invention includes DNA, such as cDNA or double or single stranded genomic DNA and RNA. In this regard, “DNA” (deoxyribonucleic acid) means any chain or sequence of the chemical building blocks adenine (A), guanine (G), cytosine (C) and thymine (T), called nucleotide bases, that are linked together on a deoxyribose sugar backbone. DNA can have one strand of nucleotide bases, or two complimentary strands which may form a double helix structure. “RNA” (ribonucleic acid) means any chain or sequence of the chemical building blocks adenine (A), guanine (G), cytosine (C) and uracil (U), called nucleotide bases that are linked together on a ribose sugar backbone. RNA typically has one strand of nucleotide bases. Included are also single- and double-stranded hybrid molecules, i.e., DNA-RNA. The nucleic acid molecule may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see Braasch and Corey, Chem Biol 2001, 8: 1). LNA is an RNA derivative in which the ribose ring is constrained by a methylene linkage between the 2′-oxygen and the 4′-carbon. Also included are nucleic acids containing modified bases, for example thio-uracil, thio-guanine and fluoro-uracil. A nucleic acid molecule typically carries genetic information, including the information used by cellular machinery to make proteins and/or polypeptides. The nucleic acid molecule of the invention may additionally comprise promoters, enhancers, response elements, signal sequences, polyadenylation sequences, introns, 5′- and 3′-non-coding regions, and the like.

The term “polypeptide” as used herein interchangeably with the term “protein” describes linear molecular chains of amino acids, including single chain proteins, containing more than 30 amino acids, whereas the term “peptide” describes linear molecular chains of amino acids, including single chain proteins, containing less than and up to 30 amino acids. Polypeptides may further form oligomers consisting of at least two identical or different molecules. The corresponding higher order structures of such multimers are, correspondingly, termed homo- or heterodimers, homo- or heterotrimers etc. The polypeptides of the invention may form heteromultimers or homomultimers, such as heterodimers or homodimers. Furthermore, peptidomimetics of such proteins/polypeptides where amino acid(s) and/or peptide bond(s) have been replaced by functional analogues are also encompassed by the invention. Such functional analogues include all known amino acids other than the 20 gene-encoded amino acids, such as selenocysteine. The terms “polypeptide” and “protein” also refer to naturally modified polypeptides and proteins where the modification is effected e.g. by glycosylation, acetylation, phosphorylation, ubiqitinylation and similar modifications which are well known in the art.

The term “a polypeptide having the activity of an endonuclease” as used herein means a polypeptide which is capable of cleaving the phosphodiester bonds between nucleotides subunits of nucleic acids within a polynucleotide chain.

According to the invention, the endonuclease enzymatic activity is considered as stable when, in the respective conditions, the enzyme is capable of lasting long enough to obtain the desired effect, namely the cleavage of its substrate. In this regard it is noted that endonuclease activity can be assayed as described in the examples of the specification or by methods well known in the art. For example, a nucleic acid molecule can be exposed to a protein whose endonuclease activity is to be assessed under conditions that are suitable for endonuclease enzymatic activity. After incubation, the composition comprising the nucleic acid molecule (with or without said protein to be assessed) may be subjected to an assay for assessing the length of a nucleic acid molecule such as, e.g., gel-electrophoresis, to determine whether the nucleic acid molecule has been cleaved.

In accordance with the present invention, the term “percent (%) sequence identity” describes the number of matches (“hits”) of identical nucleotides/amino acids of two or more aligned nucleic acid or amino acid sequences as compared to the number of nucleotides or amino acid residues making up the overall length of the template nucleic acid or amino acid sequences. In other terms, using an alignment, for two or more sequences or subsequences the percentage of amino acid residues or nucleotides that are the same (e.g. 95% identity) may be determined, when the (sub)sequences are compared and aligned for maximum correspondence over a window of comparison, or over a designated region as measured using a sequence comparison algorithm as known in the art, or when manually aligned and visually inspected. This definition also applies to the complement of any sequence to be aligned. Amino acid sequence analysis and alignment in connection with the present invention was carried out using the NCBI BLAST algorithm (Stephen F. Altschul, Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402) and the CLC main workbench software (version 5.7.1; CLC bio, Aarhus, Denmark) which are preferably employed in accordance with this invention. Preferably, the published standard parameters are used (Altschul et al. loc cit.). The skilled person is aware of additional suitable programs to align nucleic acid sequences. A preferred program for nucleic acid sequence alignment in accordance with the invention is the CLC main workbench software using the standard alignment parameters of the software program (version 5.7.1; CLC bio, Aarhus, Denmark).

As defined in the embodiments herein above, certain amino acid sequence identities are envisaged by the invention. Also envisaged are—with increasing preference—amino acid sequence identities of at least 75%, at least 80%, at least 85%, at least 90%, at least 95%, at least 97.5%, at least 98%, at least 98.5%, at least 99%, at least 99.5%, at least 99.8%, and 100% identity to the respective amino acid sequence in accordance with the invention.

As defined in the embodiments herein above, certain nucleotide sequence identities are envisaged by the invention. Also envisaged are—with increasing preference—nucleotide sequence identities of at least 55%, at least 60%, at least 65%, at least 70%, at least 75%, at least 80%, at least 85%, at least 90%, at least 95%, at least 97.5%, at least 98%, at least 98.5%, at least 99%, at least 99.5%, at least 99.8%, and 100% identity to the respective nucleic acid sequence in accordance with the invention.

It will be readily appreciated by the skilled person that more than one nucleic acid molecule may encode the same polypeptide due to the degeneracy of the genetic code. Degeneracy results because a triplet code designates 20 amino acids and a stop codon. Because four bases exist which are utilized to encode genetic information, triplet codons are required to produce at least 21 different codes. The possible 43 possibilities for bases in triplets give 64 possible codons, meaning that some degeneracy must exist. As a result, some amino acids are encoded by more than one triplet, i.e. by up to six. The degeneracy mostly arises from alterations in the third position in a triplet. This means that nucleic acid molecules having different sequences, but still encoding the same polypeptide are envisaged and can be employed in accordance with the method of present invention.

Fragments according to the present invention are polypeptides having the activity of an endonuclease as defined herein above and comprise at least 90 amino acids. In this regard, it is preferred—with increasing preference—that the fragments according the present invention are polypeptides of at least 100, at least 125, at least 150, at least 200 amino acids, at least 300 amino acids, at least 400 amino acids. Fragments of the polypeptide of the invention, which substantially retain endonuclease activity, include N-terminal truncations, C-terminal truncations, amino acid substitutions, internal deletions and addition of amino acids (either internally or at either terminus of the protein). For example, conservative amino acid substitutions are known in the art and may be introduced into the endonuclease of the invention without substantially affecting endonuclease activity, i.e. reducing said activity.

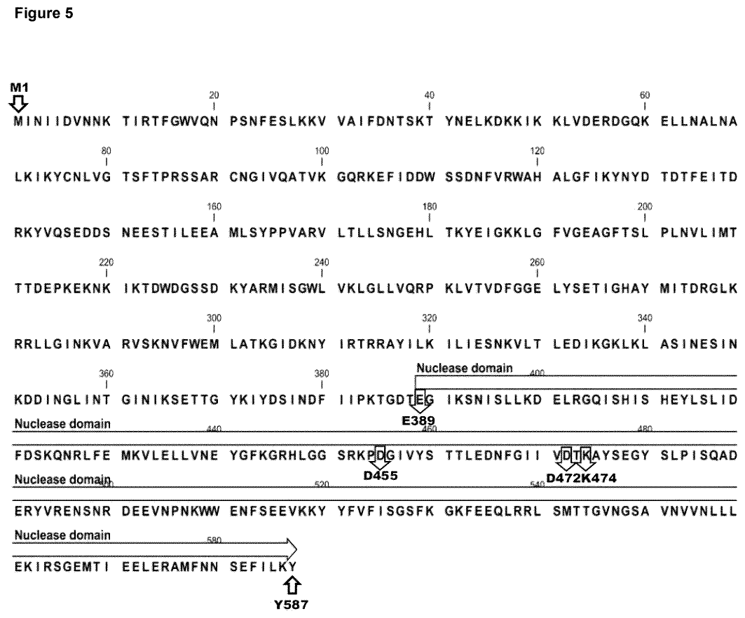

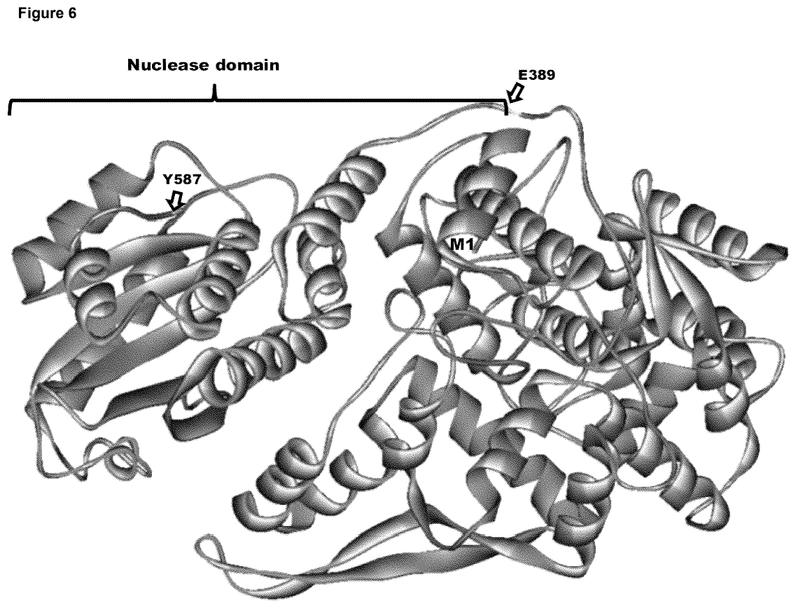

As is evident from the examples, the inventor was able to identify and isolate a novel nuclease, in particular the endonuclease domain, derived from a Clostridium strain as detailed below. Specifically, the inventor could establish the utility of the gene product of a putative bacterial gene without known functional connotation as a sequence unspecific nuclease. The novel nuclease can be employed in various experimental settings just as any other nuclease. For example, it may be used to randomly cleave nucleic acid molecules or, e.g., in fusion with DNA-binding domains, for site-specific cleavage of nucleic acid molecules. Importantly, and as outlined below and specifically in the examples, the novel endonuclease can be used in combination with TAL effector protein DNA-binding domains as part of a fusion protein for sequence-specific nucleic acid cleavage. In this respect, the novel nuclease shows its superiority over state of the art endonucleases other than FokI which could so far not be shown to be active in corresponding fusion proteins. Briefly, the inventors tested the gene product of said uncharacterised, hypothetical microbial gene which they designated as “Clo051” (SEQ ID NO: 17) and which is derived from the genome of Clostridium spec. 7 2 43FAA (NCBI Reference Sequence: ZP_05132802.1; publication/database release date: Jun. 9, 2010), more specifically its putative nuclease domain (see FIGS. 5 and 6), for its endonuclease activity in combination with the DNA-binding domain of a TAL effector protein. Also various known endonuclease proteins were tested in combination with TAL effector protein DNA binding domains as well as two more hypothetical microbial genes. Surprisingly, only the nuclease domain from Clo051 could be shown to be active, whereas the other fusion proteins did not show activity (see Example 1 for details). The comparative experiments emphasized the significance of the finding of the present invention in that a novel nuclease has been identified that also exhibits activity when fused to the DNA-binding domains of TAL effector proteins. TAL effector proteins are expressed by plant pathogens of the genus Xanthomonas and reprogram host cells by mimicking eukaryotic transcription factors. TAL effector proteins are characterized by a central domain of tandem repeats of 32 to 34 amino acid that constitute a DNA-binding domain. The number and order of repeats in a TAL effector protein determines its specific DNA binding activity. (Boch, J., et al. 2009 Science 326: 1509-12). The amino acid sequences of the repeats are conserved, except for two adjacent highly variable residues (at positions 12 and 13) that determine specificity towards the DNA base A, G, C or T. Binding to DNA is mediated by contacting a nucleotide of the DNA double helix with the variable residues at position 12 and 13 within the Tal effector motif resulting into a one-to-one correspondence between sequential repeats in the Tal effector proteins and sequential nucleotides in the target DNA. Binding to longer DNA sequences is achieved by linking several of these Tal effector motifs in tandem to form a “DNA-binding domain of a Tal effector protein”. The use of such DNA-binding domains of Tal effector proteins for the creation of Tal effector motif—nuclease fusion proteins that recognize and cleave a specific target sequence depends on the reliable creation of DNA-binding domains of Tal effector proteins that can specifically recognize said particular target. The advantage of the TAL repeat elements, as compared to e.g. zinc-finger elements, is provided by their truly modular nature. Thereby, new sequence specific DNA binding proteins can be generated through the combination of the four basic TAL elements that are specific for the A, C, G or T nucleotide.

It is important to note that in the present invention the Clo051 nuclease domain fused to DNA-binding domains of TAL effector proteins has been tested and found to be active in mammalian, specifically human cultured cells. Therefore, the utility of Clo051 nuclease domain fusion proteins for DNA and gene manipulation, specifically but without limitation in mammalian cells has been directly proven in the biological system that provides important applications for this technology. This finding is of particular importance since studies on protein function that are performed in lower eucaryotic organisms, like e.g. yeast, do not allow a definite conclusion on the utility of the protein under study in mammalian cells. For example, a specific protein may function optimal at 300 Celsius, the growth temperature of yeast, but becomes unstable or inactive at 370 Celsius as the typical body temperature of mammals. In addition, the intracellular milieu of e.g. yeast cells, like ion and protein concentration, protein diversity and protein degradation mechanisms, are distinguished from the intracellular milieu of mammalian cells.

While the examples only describe the use of the nuclease domain of Clo051 (SEQ ID NO: 1), e.g. in combination with DNA-binding domains, the skilled person will appreciate that one may also employ the entire sequence of Clo051 as set forth in SEQ ID NO: 17 or shorter fragments thereof having endonuclease activity and comprising the amino acid sequence of SEQ ID NO: 1. The amino acid sequence of SEQ ID NO: 1 starts at E389 and ends at Y587 of the amino acid of SEQ ID NO: 17 as also exemplified in FIG. 5.

In a preferred embodiment of the nucleic acid molecule of the invention, in (I)(c) in said amino acid sequence having at least 70% sequence identity to SEQ ID NO: 1 the amino acid residues P66, D67, D84 and/or K86 of SEQ ID NO: 1 are not modified.

The nuclease domain of Clo051, like many type-II restriction endonucleases and e.g. the DNA repair protein MutH, share the conserved sequence motif PD-(D/E)XK within the core of their catalytic domain. The core serves as a scaffold for a weakly conserved active site, typically comprising two or three acidic residues (Asp or Glu) and one Lys residue, which together form the hallmark bipartite catalytic motif [(P)D. Xn. (D/E)XK] (where X is any amino acid). This motif has led to naming this superfamily of proteins as ‘PD-(D/E)XK’. Work on restriction enzymes and DNA repair proteins has shown that the three catalytic residues are located close to each other on an uneven 13-hairpin. The first D is located at the beginning of the first and shorter strand, and the E and K, separated by a hydrophobic residue x, are located in the middle of the second and longer strand. The catalytic module invariably approaches DNA from the minor groove side, and the sequence-specific binding is conducted by a separate module/subdomain in the major groove. The first two carboxylates of the DEK motif coordinate the metal ions. The first D is most conserved and coordinates both metal ions, whereas the second E can be replaced by Q, D, N, H or S, and the third K can be replaced E, Q, D, S, N or T. The Lysine residue in the conserved DEK motif coordinates the nucleophilic water in conjunction with the phosphate 3′ to the scissile bond; the same Lysine is also hydrogen bonded with a carbonyl oxygen in the DNA binding module. This Lysine, which is conserved in many restriction endonucleases and is replaced by Glu or Gln in BamHl and BgIII, has been proposed as a sensor for DNA binding and a hub that couples base recognition and DNA cleavage (Lee et al. (2005). Molecular Cell 20, 155-166; Orlowski, J. and J. M. Bujnicki (2008). Nucleic Acids Res 36(11): 3552-69).

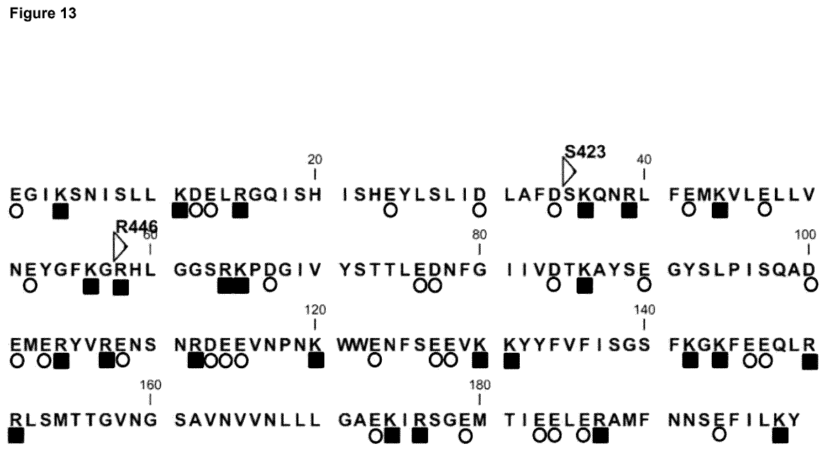

The primary sequence of the Clo051 nuclease domain between the positions E389 and Y587 of the sequence of SEQ ID NO: 17, i.e. the sequence of SEQ ID NO: 1, exhibits a unique distribution of the positively charged arginine (R) and lysine (K) residues and of negatively charged glutamate (E) and aspartate (D) residues (FIG. 13). These residues constitute a three-dimensional landscape of charges within the Clo051 domain that determines the unique tertiary structure of this nuclease, as shown in the structural model in FIG. 6. Certain replacements of polar versus non-polar residues or of non-polar residues against polar residues, e.g. at the positions S35 and/or R58 of SEQ ID NO:1 (or S423 and R446 of SEQ ID NO: 17), alter the three-dimensional structure of the protein chain and may result into an increase of the nuclease activity. Such amino acid replacements may be made by trial and error or may follow specific hypotheses on the structural and functional impact on the Clo051 nuclease domain. Alternatively, a large number of randomly mutagenised variants of the Clo051 nuclease domain coding region can be assembled in a library by mutagenic, error prone PCR. This library of mutant molecules can be tested for the presence of hyperactive nuclease variants by a phenotypic screening assay in E. coli, yeast or mammalian cells that is coupled to a functional nuclease readout, e.g. as described for the improvement of the FLP recombinase (Buchholz et al., Nat. Biotechnol. 16, 657-62, 1998). Such a functional screen for improved nuclease variants can result into the replacement of single or multiple residues that lead to increased nuclease activity as compared to the Clo051 wildtype form.

Also envisaged are embodiments where more than the amino acid residues P66, D67, D84 and/or K86 of SEQ ID NO: 1 are not modified such as, e.g., amino acid stretches as, e.g. from at least P66 to at least K86, at least R64 to at least Y88, at least G62 to at least E90, as well as L60 to at least Y92 of SEQ ID NO: 1.

In a preferred embodiment of the invention, the nucleic acid molecule further encodes a DNA-binding domain.

In this embodiment the nucleic acid molecule of the invention encodes a fusion protein having the activity of an endonuclease and comprises a DNA-binding domain and a cleavage domain comprising or consisting of the novel endonuclease domain. The term “fusion protein” is well-known in the art and has the same meaning herein. Namely, it refers to a protein generated by joining two or more target nucleic acid sequences, e.g. genes, which originally code for separate proteins to create a fusion construct. Translation of said fusion construct results in a single protein with the functional properties derived from said separate proteins. The two proteins giving rise to the fusion protein may be connected by a linker, such as, e.g., a peptide linker. In other words, the DNA-binding domain and the cleavage domain of the nucleases may be directly fused to one another or may be fused via a linker.

The term “linker” as used in accordance with the present invention relates to a sequel of amino acids (i.e. peptide linkers) as well as to non-peptide linkers.

Peptide linkers as envisaged by the present invention are peptide or polypeptide linkers of at least 1 amino acid in length. Preferably, the linkers are 1 to 100 amino acids in length. More preferably, the linkers are 5 to 50 amino acids in length and even more preferably, the linkers are 10 to 20 amino acids in length. It is well known to the skilled person that the nature, i.e. the length and/or amino acid sequence of the linker may modify or enhance the stability and/or solubility of the molecule. Thus, the length and sequence of a linker depends on the composition of the respective portions of the fusion protein.

The skilled person is aware of methods to test the suitability of different linkers. For example, the properties of the molecule can easily be tested by testing the nuclease activity as well as the DNA-binding specificity of the respective portions of the fusion protein to be used in the method of the invention.

It will be appreciated by the skilled person that when the fusion protein is provided as a nucleic acid molecule encoding the fusion protein in expressible form, the linker is a peptide linker also encoded by said nucleic acid molecule.

The term “non-peptide linker”, as used in accordance with the present invention, refers to linkage groups having two or more reactive groups but excluding peptide linkers as defined above. For example, the non-peptide linker may be a polymer having reactive groups at both ends, which individually bind to reactive groups of the individual portions of the fusion protein, for example, an amino terminus, a lysine residue, a histidine residue or a cysteine residue. The reactive groups of the polymer include an aldehyde group, a propionic aldehyde group, a butyl aldehyde group, a maleimide group, a ketone group, a vinyl sulfone group, a thiol group, a hydrazide group, a carbonyldimidazole (CDI) group, a nitrophenyl carbonate (NPC) group, a trysylate group, an isocyanate group, and succinimide derivatives. Examples of succinimide derivatives include succinimidyl propionate (SPA), succinimidyl butanoic acid (SBA), succinimidyl carboxymethylate (SCM), succinimidyl succinamide (SSA), succinimidyl succinate (SS), succinimidyl carbonate, and N-hydroxy succinimide (NHS). The reactive groups at both ends of the non-peptide polymer may be the same or different. For example, the non-peptide polymer may have a maleimide group at one end and an aldehyde group at another end. Preferably, the linker is a peptide linker. More preferably, the peptide linker consists of seven glycine residues.

Also the fusion protein may be flanked N- or C-terminally by additional sequences unrelated to said proteins in the fusion protein. In accordance with the present invention, a fusion protein of the invention comprises a DNA-binding domain. The term “DNA-binding domain” has the same meaning as known in the art and relates to a sequence motif/conformation within a protein that binds to DNA motifs. Protein domains that can specifically bind to a nucleic acid sequence include, e.g., zinc finger repeats, the helix-turn-helix (HTH) motif of homeodomains, and the ribbon-helix-helix (RHH) motif. Specific binding refers to the sequence specific binding and is specific, when a DNA-binding domain statistically only binds to a particular sequence and does not or essentially not bind to an unrelated sequence. The skilled person is well-aware of sequences encoding DNA-binding domains (Rohs et al. (2010). Annu. Rev. Biochem. 79, 233-269; Maeder et al. (2009). Nat. Protocols 10, 1471-1501).

In a more preferred embodiment of the nucleic acid molecule of the invention, the DNA-binding domain is a TAL effector motif of a TAL effector protein.

This embodiment relates to a nucleic acid molecule also encoding a TAL nuclease. The term “TAL nuclease” as used herein, is well known in the art and refers to a fusion protein comprising a DNA-binding domain, wherein the DNA-binding domain comprises or consists of Tal effector motifs of a TAL effector protein and the non-specific cleavage domain of a restriction nuclease. The fusion protein of the invention that is also employed in the method of the invention below retains or essentially retains the enzymatic activity of the endonuclease of the invention. In accordance with the present invention, said endonuclease activity (also referred to as function) is essentially retained if at least 60% of the biological activity of the endonuclease activity are retained. Preferably, at least 75% or at least 80% of the endonuclease activity are retained. More preferred is that at least 90% such as at least 95%, even more preferred at least 98% such as at least 99% of the biological activity of the endonuclease are retained. Most preferred is that the biological activity is fully, i.e. to 100%, retained. Also in accordance with the invention, fusion proteins having an increased biological activity compared to the endonuclease when not fused to a DNA-binding domain, i.e. more than 100% activity, are envisaged. Methods of assessing biological activity of (restriction) endonucleases are well known to the person skilled in the art and include, without being limiting, the incubation of an endonuclease with recombinant DNA and the analysis of the reaction products by gel electrophoresis (Bloch K D.; Curr Protoc Mol Biol 2001; Chapter 3:Unit 3.2).

The term “Tal effector protein”, as used herein, refers to proteins belonging to the TAL (transcription activator-like) family of proteins. These proteins are expressed by bacterial plant pathogens of the genus Xanthomonas. Members of the large TAL effector family are key virulence factors of Xanthomonas and reprogram host cells by mimicking eukaryotic transcription factors. The pathogenicity of many bacteria depends on the injection of effector proteins via type III secretion into eukaryotic cells in order to manipulate cellular processes. TAL effector proteins from plant pathogenic Xanthomonas are important virulence factors that act as transcriptional activators in the plant cell nucleus. PthXo1, a TAL effector protein of a Xanthomonas rice pathogen, activates expression of the rice gene Os8N3, allowing Xanthomonas to colonize rice plants. TAL effector proteins are characterized by a central domain of tandem repeats, i.e. a DNA-binding domain as well as nuclear localization signals (NLSs) and an acidic transcriptional activation domain. Members of this effector family are highly conserved and differ mainly in the amino acid sequence of their repeats and in the number of repeats. The number and order of repeats in a TAL effector protein determine its specific activity. These repeats are referred to herein as “TAL effector motifs”. One exemplary member of this effector family, AvrBs3 from Xanthomonas campestris pv. vesicatoria, contains 17.5 repeats and induces expression of UPA (up-regulated by AvrBs3) genes, including the Bs3 resistance gene in pepper plants (Kay, et al. 2005 Mol Plant Microbe Interact 18(8): 838-48; Kay, S. and U. Bonas 2009 Curr Opin Microbiol 12(1): 37-43). The repeats of AvrBs3 are essential for DNA binding of AvrBs3 and represent a distinct type of DNA binding domain. The mechanism of sequence specific DNA recognition has been elucidated by recent studies on the AvrBs3, Hax2, Hax3 and Hax4 proteins that revealed the TAL effectors' DNA recognition code (Boch, J., et al. 2009 Science 326: 1509-12).

Tal effector motifs or repeats are 32 to 34 amino acid protein sequence motifs. The amino acid sequences of the repeats are conserved, except for two adjacent highly variable residues (at positions 12 and 13) that determine specificity towards the DNA base A, G, C or T. In other words, binding to DNA is mediated by contacting a nucleotide of the DNA double helix with the variable residues at position 12 and 13 within the Tal effector motif of a particular Tal effector protein (Boch, J., et al. 2009 Science 326: 1509-12). Therefore, a one-to-one correspondence between sequential amino acid repeats in the Tal effector proteins and sequential nucleotides in the target DNA was found. Each Tal effector motif primarily recognizes a single nucleotide within the DNA substrate. For example, the combination of histidine at position 12 and aspartic acid at position 13 specifically binds cytosine; the combination of asparagine at both position 12 and position 13 specifically binds guanosine; the combination of asparagine at position 12 and isoleucine at position 13 specifically binds adenosine and the combination of asparagine at position 12 and glycine at position 13 specifically binds thymidine. Binding to longer DNA sequences is achieved by linking several of these Tal effector motifs in tandem to form a “DNA-binding domain of a Tal effector protein”. Thus, a DNA-binding domain of a Tal effector protein relates to DNA-binding domains found in naturally occurring Tal effector proteins as well as to DNA-binding domains designed to bind to a specific target nucleotide sequence as described in the examples below. The use of such DNA-binding domains of Tal effector proteins for the generation of Tal effector motif—nuclease fusion proteins that recognize and cleave a specific target sequence depends on the reliable generation of DNA-binding domains of Tal effector proteins that can specifically recognize said particular target. Methods for the generation of DNA-binding domains of Tal effector proteins are well-known in the art (Zhang et al. (2011). Nat Biotechol. 29, 149-153; Cermak et al. (2011). Nucleic Acis Res. April 14, PubMed identifier 21493687).

Preferably, the DNA-binding domain is derived from the Tal effector motifs found in naturally occurring Tal effector proteins, such as for example Tal effector proteins selected from the group consisting of AvrBs3, Hax2, Hax3 or Hax4 (Bonas et al. 1989. Mol Gen Genet 218(1): 127-36; Kay et al. 2005 Mol Plant Microbe Interact 18(8): 838-48).

Envisaged in accordance with the present invention are fusion proteins that are provided as a DNA-binding domain of a Tal effector protein coupled with a single nuclease domain. These monomeric proteins can be combined to act as a functional dimer in order to develop nuclease activity through the cooperation of two nuclease domains, each being part of one fusion protein.

Preferably, the TAL nuclease in accordance with the present invention comprises more than one, i.e. several Tal effector motifs, such as at least 12 Tal effector motifs, such as for example at least 14 or at least 16 Tal effector motifs. More preferably, the TAL nuclease comprises at least 18 Tal effector motifs. In other words, the DNA-binding domain of a Tal effector protein within said fusion protein is comprised of at least 18 Tal effector motifs. In the case of fusion proteins consisting of dimers as described above this means that each fusion protein monomer comprises at least nine Tal effector motifs. Methods for testing the DNA-binding specificity of a fusion protein in accordance with the present invention are known to the skilled person and include, without being limiting, transcriptional reporter gene assays and electrophoretic mobility shift assays (EMSA).

Preferably, the binding site of the fusion protein is up to 500 nucleotides, such as up to 250 nucleotides, up to 100 nucleotides, up to 50 nucleotides, up to 25 nucleotides, up to 10 nucleotides such as up to 5 nucleotides upstream (i.e. 5′) or downstream (i.e. 3′) of the nucleotide(s) that is/are modified in accordance with the method of the present invention as detailed below.

In another embodiment, the invention relates to a vector encoding the nucleic acid molecule of the invention.

The term “vector” in accordance with the invention preferably means a plasmid, cosmid, virus, bacteriophage or another vector used e.g. conventionally in genetic engineering which carries the nucleic acid molecule of the invention either encoding the peptide or the fusion protein of the invention. Accordingly, the nucleic acid molecule of the invention may be inserted into several commercially available vectors. Non-limiting examples include prokaryotic plasmid vectors, such as of the pUC-series, pBluescript (Stratagene), the pET-series of expression vectors (Novagen) or pCRTOPO (Invitrogen) and vectors compatible with an expression in mammalian cells like pREP (Invitrogen), pcDNA3 (Invitrogen), pCEP4 (Invitrogen), pMC1 neo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, pLXIN, pSIR (Clontech), pIRES-EGFP (Clontech), pEAK-10 (Edge Biosystems) pTriEx-Hygro (Novagen) and pClNeo (Promega). Examples for plasmid vectors suitable for Pichia pastoris comprise e.g. the plasmids pAO815, pPIC9K and pPIC3.5K (all Intvitrogen).

The nucleic acid molecule of the present invention referred to above may also be inserted into vectors such that a (further) translational fusion with another nucleic acid molecule is generated. To this aim, overlap extension PCR can be applied (e.g. Wurch, T., Lestienne, F., and Pauwels, P. J., A modified overlap extension PCR method to create chimeric genes in the absence of restriction enzymes, Biotechn. Techn. 12, 9, Sep. 1998, 653-657). The products arising therefrom are termed fusion proteins and will be described further below. The other nucleic acid molecules may encode a protein which may e.g. increase the solubility and/or facilitate the purification of the protein encoded by the nucleic acid molecule of the invention. Non-limiting examples include pET32, pET41, pET43. The vectors may also contain an additional expressible nucleic acid coding for one or more chaperones to facilitate correct protein folding. Suitable bacterial expression hosts comprise e.g. strains derived from BL21 (such as BL21(DE3), BL21(DE3)PlysS, BL21(DE3)RIL, BL21(DE3)PRARE) or Rosetta®.

Particularly preferred plasmids which can be used to introduce the nucleic acid encoding the polypeptide of the invention having the activity of an endonuclease into the host cell are: pUC18/19 (Roche Biochemicals), pBluescript II (Alting-Mees, et al. (1992). Meth. Enzymol., 216, 483-495), pKK-177-3H (Roche Biochemicals), pBTac2 (Roche Biochemicals), pKK223-3 (Amersham Pharmacia Biotech), pKK-233-3 (Stratagene) and pET (Novagen).

For vector modification techniques, see Sambrook and Russel, 2001. Generally, vectors can contain one or more origins of replication (ori) and inheritance systems for cloning or expression, one or more markers for selection in the host, e.g., antibiotic resistance, and one or more expression cassettes. Suitable origins of replication include, for example, the Col E1, the SV40 viral and the M13 origins of replication.

The coding sequences inserted in the vector can e.g. be synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression in prokaryotes or eukaryotic cells are well known to those skilled in the art. These elements comprise regulatory sequences ensuring the initiation of the transcription (e.g., translation initiation codon, transcriptional termination sequences, promoters, enhancers, and/or insulators), internal ribosomal entry sites (IRES) and optionally poly-A signals ensuring termination of transcription and stabilization of the transcript. Additional regulatory elements may include transcriptional as well as translational enhancers, and/or naturally-associated or heterologous promoter regions. The regulatory elements may heterologous regulatory elements. Preferably, the nucleic acid molecule of the invention is operably linked to such expression control sequences allowing expression in prokaryotes or eukaryotic cells. The vector may further comprise nucleotide sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the nucleic acid molecule of the invention. Such leader sequences are well known in the art. Specifically designed vectors allow the shuttling of DNA between different hosts, such as bacteria-fungal cells or bacteria-animal cells.

The co-transfection with a selectable marker such as kanamycin or ampicillin resistance genes for culturing in E. coli and other bacteria allows the identification and isolation of the transfected cells. Selectable markers for mammalian cell culture are the dhfr, gpt, neomycin, hygromycin resistance genes. The transfected nucleic acid can also be amplified to express large amounts of the encoded polypeptide. The DHFR (dihydrofolate reductase) marker is useful to develop cell lines that carry several hundred or even several thousand copies of the gene of interest. Another useful selection marker is the enzyme glutamine synthase (GS) (Fisher et al., Infect Immun. 1991 October; 59(10):3562-5; Bebbington et al., Biotechnology (NY). 1992 February; 10(2):169-75).

Using such markers, the cells are grown in selective medium and the cells with the highest resistance are selected.

In another embodiment the invention relates to a host cell comprising, e.g., as a result of transformation, transduction, microinjection or transfection, the nucleic acid molecule or the vector of the invention.

A variety of host-expression systems may be conceived to express the endonuclease coding sequence in a host cell using a suitable vector.

The “host cell” in accordance with the invention may be produced by introducing the nucleic acid molecule or vector(s) of the invention into the host cell which upon its/their presence preferably mediates the expression of the nucleic acid molecule of the invention encoding the endonuclease of the invention. The host from which the host cell is derived may be any prokaryote or eukaryotic cell.

A suitable eukaryotic host cell may be a vertebrate cell, an amphibian cell, a fish cell, an insect cell, a fungal/yeast cell, a nematode cell or a plant cell. The insect cell may be a Spodoptera frugiperda cell, a Drosophila S2 cell or a Spodoptera Sf9 cell, the fungal/yeast cell may a Saccharomyces cerevisiae cell, Pichia pastoris cell or an Aspergillus cell. It is preferred that the vertebrate cell is a mammalian cell such as a human cell, CHO, COS, 293 or Bowes melanoma cell. The plant cell is preferably selected independently from a cell of Anacardium, Anona, Arachis, Artocarpus, Asparagus, Atropa, Avena, Brassica, Carica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoseyamus, Lactuca, Linum, Lolium, Lupinus, Lycopersicon, Malus, Manihot, Majorana, Medicago, Nicotiana, Olea, Oryza, Panieum, Pannesetum, Passiflora, Persea, Phaseolus, Pistachia, Pisum, Pyrus, Prunus, Psidium, Raphanus, Ricinus, Secale, Senecio, Sinapis, Solanum, Sorghum, Theobromus, Trigonella, Triticum, Vicia, Vitis, Vigna and Zea. The cell may be a part of a cell line. The cell from plant may, e.g., be derived from root, leave, bark, needle, bole or caulis.

Suitable prokaryotes (bacteria) useful as hosts for the invention are those generally used for cloning and/or expression like E. coli (e.g., E. coli strains BL21, HB101, DH5a, XL1 Blue, Y1090 and JM101), Salmonella typhimurium, Serratia marcescens, Burkholderia glumae, Pseudomonas putida, Pseudomonas fluorescens, Pseudomonas stutzeri, Streptomyces lividans, Lactococcus lactis, Mycobacterium smegmatis, Streptomyces or Bacillus subtilis. Appropriate culture mediums and conditions for the above described host cells are known in the art.

Preferred examples for host cell to be genetically engineered with the nucleic acid molecule or the vector(s) of the invention is a cell of yeast, E. coli and/or a species of the genus Bacillus (e.g., B. subtilis). The most preferred host cell is Bacillus spec.

In a further embodiment the invention relates to a method of producing a protein or fusion having the activity of an endonuclease as defined herein above comprising the steps: (a) culturing the host cell of the invention and (b) isolating the produced protein or fusion protein having the activity of said endonuclease.

Suitable conditions for culturing a prokaryotic or eukaryotic host are well known to the person skilled in the art. Suitable conditions for culturing E. coli DH18BAkat E (Invitrogen), Pichia pastoris or Aspergillus niger are, for example provided in the examples of the invention. In general, suitable conditions for culturing bacteria are growing them under aeration in Luria Bertani (LB) medium. To increase the yield and the solubility of the expression product, the medium can be buffered or supplemented with suitable additives known to enhance or facilitate both. E. coli can be cultured from 4 to about 37° C., the exact temperature or sequence of temperatures depends on the molecule to be overexpressed. In general, Aspergillus sp. may be grown on Sabouraud dextrose agar, or potato dextrose agar at about to 10° C. to about 40° C., and preferably at about 25° C. Suitable conditions for yeast cultures are known, for example from Guthrie and Fink, “Guide to Yeast Genetics and Molecular Cell Biology” (2002); Academic Pr Inc. The skilled person is also aware of all these conditions and may further adapt these conditions to the needs of a particular host species and the requirements of the polypeptide expressed. In case an inducible promoter controls the nucleic acid of the invention in the vector present in the host cell, expression of the polypeptide can be induced by addition of an appropriate inducing agent. Suitable expression protocols and strategies are known to the skilled person.

Depending on the cell type and its specific requirements, mammalian cell culture can e.g. be carried out in RPMI or DMEM medium containing 10% (v/v) FCS, 2 mM L-glutamine and 100 U/ml penicillin/streptomycin. The cells can be kept at 37° C. in a 5% 002, water saturated atmosphere.

Suitable expression protocols for eukaryotic cells are well known to the skilled person and can be retrieved e.g. from in Sambrook, 2001.

Methods of isolation of the polypeptide produced are well-known in the art and comprise without limitation method steps such as ion exchange chromatography, gel filtration chromatography (size exclusion chromatography), affinity chromatography, high pressure liquid chromatography (HPLC), reversed phase HPLC, disc gel electrophoresis or immunoprecipitation, see, for example, in Sambrook, 2001.

The step of protein isolation is preferably a step of protein purification. Protein purification in accordance with the invention specifies a process or a series of processes intended to further isolate the polypeptide of the invention from a complex mixture preferably to homogeneity. Purification steps, for example, exploit differences in protein size, physico-chemical properties and binding affinity. For example, proteins may be purified according to their isoelectric points by running them through a pH graded gel or an ion exchange column. Further, proteins may be separated according to their size or molecular weight via size exclusion chromatography or by SDS-PAGE (sodium dodecyl sulfate-polyacrylamide gel electrophoresis) analysis. In the art, proteins are often purified by using 2D-PAGE and are then further analysed by peptide mass fingerprinting to establish the protein identity. This is very useful for scientific purposes and the detection limits for protein are very low and nanogram amounts of protein are sufficient for their analysis. Proteins may also be separated by polarity/hydrophobicity via high performance liquid chromatography or reversed-phase chromatography. Thus, methods for protein purification are well known to the skilled person.

Furthermore, the invention relates in one embodiment to a protein or fusion protein having the activity of an endonuclease encoded by the nucleic acid molecule or vector of the invention.

The definitions for proteins or fusion proteins having the activity of an endonuclease encoded by the nucleic acid molecule or vector of the invention already given in the above embodiments pertaining to the nucleic acid molecule or vector of the invention apply explicitly also to this embodiment.

As a consequence of its endonuclease activity, another embodiment of the invention relates to the use of the protein or fusion protein of the invention to cleave a nucleic acid molecule, e.g. in one of the methods of the invention described below.

Furthermore, the present invention also relates to a kit comprising the nucleic acid molecule, the protein and/or the fusion protein of the invention. The various components of the kit may be packaged in one or more containers such as one or more vials. The vials may, in addition to the components, comprise preservatives or buffers for storage. In addition, the kit may contain instructions for use.

In another embodiment, the invention relates to a method of modifying a target sequence in the genome of a eukaryotic cell, the method comprising the step: (a) introducing into said cell the nucleic acid molecule, the vector or the protein or fusion protein of the invention.

The term “modifying” as used in accordance with the present invention refers to random and site-specific genomic manipulations resulting in changes in the nucleotide sequence of the genome of the eukaryotic host. When the fusion protein of the invention is introduced, site-specific modification of said “target sequence” in the genome is achieved via the DNA-binding domain. When only the protein of the invention is introduced, the “target sequence” is no specific sequence, because the novel endonuclease is not site-specific. Thus, the protein of the invention may be used to introduce random mutations into a genome, i.e. the “target sequence” occurs multiple times within the genome and does not depend on a specific sequence motif. The genetic material comprising these changes in its nucleotide sequence is also referred to herein as the “modified target sequence” when modification is site-specific as, e.g. in the case of using the fusion protein of the invention. The term “modifying” includes, but is not limited to, substitution, insertion and deletion of one or more nucleotides within the target sequence. In the process of homologous recombination, the end product may reflect a deletion of sequences. As is understood by the skilled person, a homologous recombination, on the other hand, always also includes the incorporation of genetic material from the donor DNA sequence, which in this embodiment, however, leads to an overall deletion. It is understood by the skilled person that by simply introducing double-strand breaks into the genome of a cell modifications can be introduced that are the result of homologous recombination (in the presence and absence of exogenous donor sequences) or an endogenous DNA-repair mechanism such as, e.g., the non-homologous end joining (NHEJ) DNA repair that is prone to introducing small deletions at the site of the double-strand break in the course of ligating the broken ends.

The term “substitution”, as used herein, refers to the replacement of nucleotides with other nucleotides. The term includes for example the replacement of single nucleotides resulting in point mutations. Said point mutations can lead to an amino acid exchange in the resulting protein product but may also not be reflected on the amino acid level. Also encompassed by the term “substitution” are mutations resulting in the replacement of multiple nucleotides, such as for example parts of genes, such as parts of exons or introns as well as replacement of entire genes.

The term “insertion” in accordance with the present invention refers to the incorporation of one or more nucleotides into a nucleic acid molecule. Insertion of parts of genes, such as parts of exons or introns as well as insertion of entire genes is also encompassed by the term “insertion”. When the number of inserted nucleotides is not dividable by three, the insertion can result in a frameshift mutation within a coding sequence of a gene. Such frameshift mutations will alter the amino acids encoded by a gene following the mutation. In some cases, such a mutation will cause the active translation of the gene to encounter a premature stop codon, resulting in an end to translation and the production of a truncated protein. When the number of inserted nucleotides is instead dividable by three, the resulting insertion is an “in-frame insertion”. In this case, the reading frame remains intact after the insertion and translation will most likely run to completion if the inserted nucleotides do not code for a stop codon. However, because of the inserted nucleotides, the resulting protein will contain, depending on the size of the insertion, one or multiple new amino acids that may affect the function of the protein.

The term “deletion” as used in accordance with the present invention refers to the loss of nucleotides or part of genes, such as exons or introns as well as entire genes. As defined with regard to the term “insertion”, the deletion of a number of nucleotides that is not evenly dividable by three will lead to a frameshift mutation, causing all of the codons occurring after the deletion to be read incorrectly during translation, potentially producing a severely altered and most likely non-functional protein. If a deletion does not result in a frameshift mutation, i.e. because the number of nucleotides deleted is dividable by three, the resulting protein is nonetheless altered as the it will lack, depending on the size of the deletion, several amino acids that may affect or effect the function of the protein.

The above defined modifications are not restricted to coding regions in the genome, but can also occur in non-coding regions of the target genome, for example in regulatory regions such as promoter or enhancer elements or in introns.

Examples of modifications of the target genome include, without being limiting, the introduction of mutations into a wild type gene in order to analyse its effect on gene function; the replacement of an entire gene with a mutated gene or, alternatively, if the target sequence comprises mutation(s), the alteration of these mutations to identify which mutation is causative of a particular effect; the removal of entire genes or proteins or the removal of regulatory elements from genes or proteins as well as the introduction of fusion-partners, such as for example purification tags such as the his-tag or the tap-tag etc. In the latter case, the term “addition” may also be used instead of “insertion” so as to describe the preferable addition of a tag to a terminus of a polypeptide rather than within the sequence of a polypeptide

The term “eukaryotic cell” as used herein, refers to any cell of a unicellular or multicellular eukaryotic organism, including cells from animals like vertebrates and from fungi and plants. Preferably, but without limitation, the cell is a mammalian cell. The term “mammalian cell” as used herein, is well known in the art and refers to any cell belonging to an animal that is grouped into the class of mammalia. The term “cell” as used in connection with the present invention can refer to a single and/or isolated cell or to a cell that is part of a multicellular entity such as a tissue, an organism or a cell culture another. In other words the method can be performed in vivo, ex vivo or in vitro. Depending on the particular goal to be achieved through modifying the genome of a mammalian cell, cells of different mammalian subclasses such as prototheria or theria may be used. For example, within the subclass of theria, preferably cells of animals of the infraclass eutheria, more preferably of the order primates, artiodactyla, perissodactyla, rodentia and lagomorpha are used in the method of the invention as detailed below. Furthermore, within a species one may choose a cell to be used in the method of the invention based on the tissue type and/or capacity to differentiate equally depending on the goal to be achieved by modifying the genome. Three basic categories of cells make up the mammalian body: germ cells, somatic cells and stem cells. A germ cell is a cell that gives rise to gametes and thus is continuous through the generations. Stem cells can divide and differentiate into diverse specialized cell types as well as self renew to produce more stem cells. In mammals there are two main types of stem cells: embryonic stem cells and adult stem cells. Somatic cells include all cells that are not a gametes, gametocytes or undifferentiated stem cells. The cells of a mammal can also be grouped by their ability to differentiate. A totipotent (also known as omnipotent) cell is a cell that is able to differentiate into all cell types of an adult organism including placental tissue such as a zygote (fertilized oocyte) and subsequent blastomeres, whereas pluripotent cells, such as embryonic stem cells, cannot contribute to extraembryonic tissue such as the placenta, but have the potential to differentiate into any of the three germ layers endoderm, mesoderm and ectoderm. Multipotent progenitor cells have the potential to give rise to cells from multiple, but limited number of cell lineages. Further, there are oligopotent cells that can develop into only a few cell types and unipotent cells (also sometimes termed a precursor cell) that can develop into only one cell type. There are four basic types of tissues: muscle tissue, nervous tissue, connective tissue and epithelial tissue that a cell to be used in the method of the invention can be derived from, such as for example hematopoietic stem cells or neuronal stem cells. To the extent human cells are envisaged for use in the method of the invention, it is preferred that such human cell is not obtained from a human embryo, in particular not via methods entailing destruction of a human embryo. On the other hand, human embryonic stem cells are at the skilled person's disposal such as taken from existent embryonic stem cell lines commercially available. Accordingly, the present invention may be worked with human embryonic stem cells without any need to use or destroy a human embryo. Alternatively, or instead of human embryonic stem cells, pluripotent cells that resemble embryonic stem cells such induced pluripotent stem (iPS) cells may be used, the generation of which is state of the art (Hargus G et al., Proc Natl Acad Sci USA 107:15921-15926; Jaenisch R. and Young R., 2008, Cell 132:567-582; Saha K, and Jaenisch R., 2009, Cell Stem Cell 5:584-595).

The term “nucleic acid molecules encoding said protein or fusion protein in expressible form” refers to a nucleic acid molecule which, upon expression in a cell or a cell-free system, results in a functional protein or fusion protein of the invention. Preferably, but without limitation, said nucleic acid is mRNA. Alternatively, DNA having appropriate transcription signals to enable expression or cDNA may be used.

Introduction of the protein, fusion protein or of the nucleic acid molecule encoding said protein, fusion protein in expressible form into a cell can be achieved by methods known in the art and depends on the nature of said proteins or nucleic acid molecules. For example, and in the case of introducing nucleic acid molecules, said introducing can be achieved by chemical based methods (calcium phosphate, liposomes, DEAE-dextrane, polyethylenimine, nucleofection), non chemical methods (electroporation, sonoporation, optical transfection, gene electrotransfer, hydrodynamic delivery), particle-based methods (gene gun, magnetofection, impalefection) and viral methods. Preferably, the nucleic acid molecules are to be introduced into the nucleus by methods such as, e.g., microinjection or nucleofection. Methods for carrying out microinjection are well known in the art and are described for example in Nagy et al. (Nagy A, Gertsenstein M, Vintersten K, Behringer R., 2003. Manipulating the Mouse Embryo. Cold Spring Harbour, New York: Cold Spring Harbour Laboratory Press) as well as in the examples herein below. It is understood by the skilled person that depending on the method of introduction it may be advantageous to adapt DNA molecules. For example, a linear DNA molecule may be more efficient in homologous recombination events when using electroporation as method to introduce said DNA molecule into a, e.g., mammalian cell, whereas a circular DNA molecule may be more advantageous when injecting cells.

All the definitions and preferred embodiments defined above with regard to the nucleic acid molecule, protein or fusion protein of the invention also apply mutatis mutandis in the context of the method of the invention.

In accordance with the present invention, the term “target sequence in the genome” refers to the genomic location that is to be modified by the method of the invention. The “target sequence in the genome” comprises but is not restricted to the nucleotide(s) subject to the particular modification. Furthermore, and preferably with regard to the fusion protein of the invention the term “target sequence in the genome” also comprises regions for binding of homologous sequences of a second nucleic acid molecule. In other words, the term “target sequence in the genome” also comprises the sequence flanking/surrounding the relevant nucleotide(s) to be modified. In some instances, the term “target sequence” may also refer to the entire gene to be modified.

Specific binding has been defined herein above and ensures that double-strand breaks are only introduced within said target sequence.

In a more preferred embodiment of the method of the invention, the modification of said target sequence is by homologous recombination with a donor nucleic acid sequence, further comprising the step: (b) introducing a nucleic acid molecule into said cell, wherein said nucleic acid molecule comprises said donor nucleic acid sequence, wherein said donor DNA sequence is flanked upstream by a first flanking element and downstream by a second flanking element, wherein said first and second flanking element are different and wherein each of said first and second flanking element are homologous to a continuous DNA sequence on either side of the double-strand break introduced in (a) of the method of the invention within said target sequence in the genome of said eukaryotic cell.

The term “homologous recombination”, is used according to the definitions provided in the art. Thus, it refers to a mechanism of genetic recombination in which two DNA strands comprising similar nucleotide sequences exchange genetic material. Cells use homologous recombination during meiosis, where it serves to rearrange DNA to create an entirely unique set of haploid chromosomes, but also for the repair of damaged DNA, in particular for the repair of double strand breaks. The mechanism of homologous recombination is well known to the skilled person and has been described, for example by Paques and Haber (Paques F, Haber J E.; Microbiol Mol Biol Rev 1999; 63:349-404). In the method of the present invention, homologous recombination of the donor sequence is enabled by the presence of said first and said second flanking element being placed upstream (5′) and downstream (3′), respectively, of said donor DNA sequence each of which being homologous to a continuous DNA sequence within said target sequence.

In accordance with the present invention, the term “donor DNA sequence” refers to a DNA sequence that serves as a template in the process of homologous recombination and that carries the modification that is to be introduced into the target sequence. By using this donor DNA sequence as a template, the genetic information, including the modifications, is copied into the target sequence within the genome of the cell by way of homologous recombination. In non-limiting examples, the donor nucleic acid sequence can be essentially identical to the part of the target sequence to be replaced, with the exception of one nucleotide which differs and results in the introduction of a point mutation upon homologous recombination or it can consist of an additional gene previously not present in the target sequence. Conceivably, the nature, i.e. its length, base composition, similarity with the target sequence, of the donor DNA sequence depends on how the target sequence is to be modified as well as the particular goal to be achieved by the modification of the target sequence. It is understood by those skilled in the art that said donor DNA sequence is flanked by sequences that are homologous to sequences within the target sequence to enable homologous recombination to take place leading to the incorporation of the donor DNA sequence into the genome of said cell. In addition to being homologous to a continuous DNA sequence within the genomic DNA, the first and the second flanking element are different to allow targeted homologous recombination to take place.

The term “homologous to a continuous DNA sequence on either side of the double-strand break introduced in (a) of the method of the invention within said target sequence”, in accordance with the present invention, refers to regions having sufficient sequence identity to ensure specific binding to the target sequences that lie upstream and downstream of the location of the double-strand break. The term “homologous” as used herein can be interchanged with the term “identical” as outlined herein elsewhere with regard to varying levels of sequence identity. Methods to evaluate the identity level between two nucleic acid sequences are well known in the art and have been described herein above. These methods involving programs, in addition to providing a pairwise sequence alignment, also report the sequence identity level (usually in percent identity) and the probability for the occurrence of the alignment by chance (P-value) and can further be used to predict the occurrence of specific binding.

Preferably, said first and second flanking element being “homologous to a continuous DNA sequence within said target sequence” (also referred to as “homology arms” in the art) have a sequence identity with the corresponding part of the target sequence of at least 95%, more preferred at least 97%, more preferred at least 98%, more preferred at least 99%, even more preferred at least 99.9% and most preferred 100%. The above defined sequence identities are defined only with respect to those parts of the target sequence which serve as binding sites for the homology arms, i.e. said first and said second flanking element. Thus, the overall sequence identity between the entire target sequence and the homologous regions of the nucleic acid molecule of step (b) of the method of modifying a target sequence of the present invention can differ from the above defined sequence identities, due to the presence of the part of the target sequence which is to be replaced by the donor DNA sequence.

The flanking elements homologous to the target sequence comprised in the DNA molecule have a length of at least 170 bp each. Preferably, the elements each have a length of at least 250 nucleotides, at least 300 nucleotides, at least 400 nucleotides, at least 500 nucleotides, such as at least 600 nucleotides, at least 750 bp nucleotides, more preferably at least 1000 nucleotides, such as at least 1500 nucleotides, even more preferably at least 2000 nucleotides and most preferably at least 2500 nucleotides. The maximum length of the elements homologous to the target sequence comprised in the nucleic acid molecule depends on the type of cloning vector used and can be up to a length 20.000 nucleotides each in E. coli high copy plasmids using the col EI replication origin (e.g. pBluescript) or up to a length of 300.000 nucleotides each in plasmids using the F-factor origin (e.g. in BAC vectors such as for example pTARBAC1).

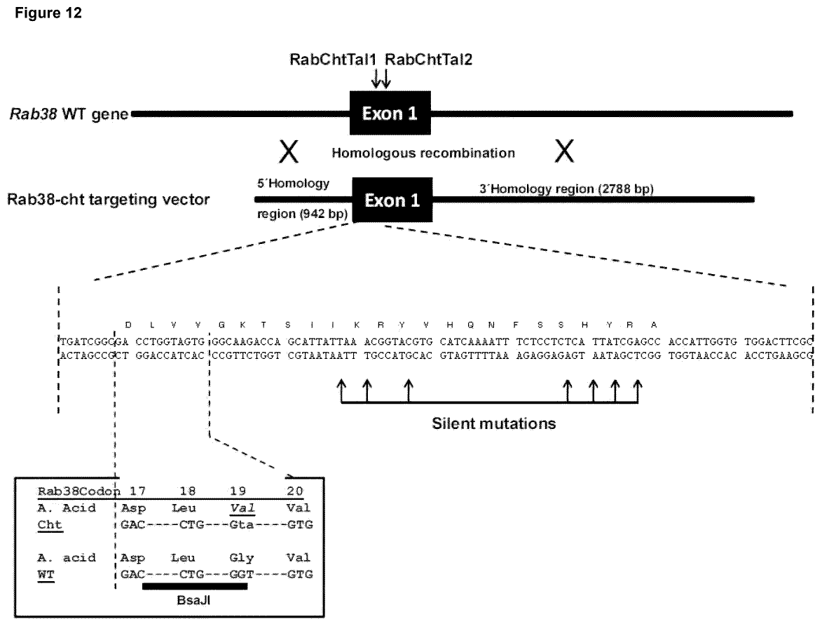

The DNA molecules comprising the donor DNA sequence and the flanking elements are necessarily if the site-specific nuclease (fusion protein) binding site is contained undisrupted within one of the flanking elements and preferably if the site-specific nuclease (fusion protein) binding site is disrupted by the donor sequence, i.e. one part on each of the flanking elements—modified so that the fusion protein not introduce a double-strand break into the sequence of the donor DNA as part of a DNA molecule. When the fusion protein is a TAL or zinc-finger nuclease, this can be achieved, e.g., by modifying either the binding or cleavage motif (see Example 2, FIG. 12).

It will be appreciated by one of skill in the art that said DNA molecule to be introduced into the cell in item (b) of the method of the invention may comprise all a nucleic acid molecule (sequence) encoding said fusion protein in expressible form and the nucleic acid molecule comprising the donor nucleic acid sequence and the flanking elements homologous to the target sequence. Alternatively, the nucleic acid molecule of item (b) may be a distinct nucleic acid molecule, to be introduced in addition to the nucleic acid molecules encoding said fusion protein in expressible form of item (a).

Also envisaged in a preferred embodiment of the method of the invention is that said cell is analysed for successful modification of said target sequence in the genome.

Methods for analysing for the presence or absence of a modification are well known in the art and include, without being limiting, assays based on physical separation of nucleic acid molecules, sequencing assays as well as cleavage and digestion assays and DNA analysis by the polymerase chain reaction (PCR).

Examples for assays based on physical separation of nucleic acid molecules include without limitation MALDI-TOF, denaturating gradient gel electrophoresis and other such methods known in the art, see for example Petersen et al., Hum. Mutat. 20 (2002) 253-259; Hsia et al., Theor. Appl. Genet. 111 (2005) 218-225; Tost and Gut, Clin. Biochem. 35 (2005) 335-350; Palais et al., Anal. Biochem. 346 (2005) 167-175.

Examples for sequencing assays comprise without limitation approaches of sequence analysis by direct sequencing, fluorescent SSCP in an automated DNA sequencer and pyrosequencing. These procedures are common in the art, see e.g. Adams et al. (Ed.), “Automated DNA Sequencing and Analysis”, Academic Press, 1994; Alphey, “DNA Sequencing: From Experimental Methods to Bioinformatics”, Springer Verlag Publishing, 1997; Ramon et al., J. Transl. Med. 1 (2003) 9; Meng et al., J. Clin. Endocrinol. Metab. 90 (2005) 3419-3422.

Examples for cleavage and digestion assays include without limitation restriction digestion assays such as restriction fragments length polymorphism assays (RFLP assays), RNase protection assays, assays based on chemical cleavage methods and enzyme mismatch cleavage assays, see e.g. Youil et al., Proc. Natl. Acad. Sci. U.S.A. 92 (1995) 87-91; Todd et al., J. Oral Maxil. Surg. 59 (2001) 660-667; Amar et al., J. Clin. Microbiol. 40 (2002) 446-452.

Alternatively, instead of analysing the cells for the presence or absence of the desired modification, in particular in the case of sequence-specific modification, successfully modified cells may be selected by incorporation of appropriate selection markers. Selection markers include positive and negative selection markers, which are well known in the art and routinely employed by the skilled person. Non-limiting examples of selection markers include dhfr, gpt, neomycin, hygromycin, dihydrofolate reductase, G418 or glutamine synthase (GS) (Murphy et al., Biochem J. 1991, 227:277; Bebbington et al., Bio/Technology 1992, 10:169). Using these markers, the cells are grown in selective medium and the cells with the highest resistance are selected. Also envisaged are combined positive-negative selection markers, which may be incorporated into the target genome by homologous recombination or random integration. After positive selection, the first cassette comprising the positive selection marker flanked by recombinase recognition sites is exchanged by recombinase mediated cassette exchange against a second, marker-less cassette. Clones containing the desired exchange cassette are then obtained by negative selection.

In a preferred embodiment of the method of the invention, the cell is selected from the group consisting of a mammalian or vertebrate cell, a plant cell or a fungal cell.

In another preferred embodiment of the method of the invention, the cell is an oocyte.

As used herein the term “oocyte” refers to the female germ cell involved in reproduction, i.e. the ovum or egg cell. In accordance with the present invention, the term “oocyte” comprises both oocytes before fertilisation as well as fertilised oocytes, which are also called zygotes. Thus, the oocyte before fertilisation comprises only maternal chromosomes, whereas an oocyte after fertilisation comprises both maternal and paternal chromosomes. After fertilisation, the oocyte remains in a double-haploid status for several hours, in mice for example for up to 18 hours after fertilisation. In accordance with the invention, the oocyte may be non-human.

In a more preferred embodiment of the method of the invention, the oocyte is a fertilised oocyte. The term “fertilised oocyte”, as used herein, refers to an oocyte after fusion with the fertilizing sperm. For a period of many hours (such as up to 18 hours in mice) after fertilisation, the oocyte is in a double-haploid state, comprising one maternal haploid pronucleus and one paternal haploid pronucleus. After migration of the two pronuclei together, their membranes break down, and the two genomes condense into chromosomes, thereby reconstituting a diploid organism. Preferably, the mammalian or avian oocyte used in the method of the present invention is a fertilised mammalian or avian oocyte in the double-haploid state.

In the case of oocytes to be used as cells in the method of the invention the protein, fusion protein or the nucleic acid molecule encoding said protein or fusion protein is introduced into the oocyte by microinjection. Microinjection into the oocyte can be carried out by injection into the nucleus (before fertilisation), the pronucleus (after fertilisation) and/or by injection into the cytoplasm (both before and after fertilisation). When a fertilised oocyte is employed, injection into the pronucleus is carried out either for one pronucleus or for both pronuclei. Injection of the Tal-finger nuclease or of a DNA encoding the Tal-finger nuclease of step (a) of the method of modifying a target sequence of the present invention is preferably into the nucleus/pronucleus, while injection of an mRNA encoding the Tal-finger nuclease of step (a) is preferably into the cytoplasm. Injection of the nucleic acid molecule of step (b) is preferably into the nucleus/pronucleus. However, injection of the nucleic acid molecule of step (b) can also be carried out into the cytoplasm when said nucleic acid molecule is provided as a nucleic acid sequence having a nuclear localisation signal to ensure delivery into the nucleus/pronucleus. Preferably, the microinjection is carried out by injection into both the nucleus/pronucleus and the cytoplasm. For example, the needle can be introduced into the nucleus/pronucleus and a first amount of the Tal-finger nuclease and/or nucleic acid molecule are injected into the nucleus/pronucleus. While removing the needle from the oocyte, a second amount of the Tal-finger nuclease and/or nucleic acid molecule is injected into the cytoplasm.

Methods for carrying out microinjection are well known in the art and are described for example in Nagy et al. (Nagy A, Gertsenstein M, Vintersten K, Behringer R., 2003. Manipulating the Mouse Embryo. Cold Spring Harbour, New York: Cold Spring Harbour Laboratory Press) as well as in the examples herein below.

Also preferred is that the nucleic acid molecule of step (b) of the method of the invention is (also) introduced into the cell by microinjection.

In another embodiment, the invention relates to method of producing a non-human vertebrate or mammal carrying a modified target sequence in its genome, the method comprising transferring a cell produced by the method of the invention into a pseudo pregnant female host.

In accordance with the present invention, the term “transferring a cell produced by the method of the invention into a pseudopregnant female host” includes the transfer of a fertilised oocyte but also the transfer of pre-implantation embryos of for example the 2-cell, 4-cell, 8-cell, 16-cell and blastocyst (70- to 100-cell) stage. Said pre-implantation embryos can be obtained by culturing the cell under appropriate conditions for it to develop into a pre-implantation embryo. Furthermore, injection or fusion of the cell with a blastocyst are appropriate methods of obtaining a pre-implantation embryo. Where the cell produced by the method of the invention is a somatic cell, derivation of induced pluripotent stem cells is required prior to transferring the cell into a female host such as for example prior to culturing the cell or injection or fusion of the cell with a pre-implantation embryo. Methods for transferring an oocyte or pre-implantation embryo to a pseudo pregnant female host are well known in the art and are, for example, described in Nagy et al., (Nagy A, Gertsenstein M, Vintersten K, Behringer R., 2003. Manipulating the Mouse Embryo. Cold Spring Harbour, New York: Cold Spring Harbour Laboratory Press).

It is further envisaged in accordance with the method of producing a non-human vertebrate or mammal carrying a modified target sequence in its genome that a step of analysis of successful genomic modification is carried out before transplantation into the female host. As a non-limiting example, the oocyte can be cultured to the 2-cell, 4-cell or 8-cell stage and one cell can be removed without destroying or altering the resulting embryo. Analysis for the genomic constitution, e.g. the presence or absence of the genomic modification, can then be carried out using for example PCR or southern blotting techniques or any of the methods described herein above. Such methods of analysis of successful genotyping prior to transplantation are known in the art and are described, for example in Peippo et al. (Peippo J, Viitala S, Virta J, Raty M, Tammiranta N, Lamminen T, Aro J, Myllymaki H, Vilkki J.; Mol Reprod Dev 2007; 74:1373-1378).

Where the cell is an oocyte, the method of producing a non-human vertebrate or mammal carrying a modified target sequence in its genome comprises (a) modifying the target sequence in the genome of a vertebrate or mammalian oocyte in accordance with the method of the invention; (b) transferring the oocyte obtained in (a) to a pseudopregnant female host; and, optionally, (c) analysing the offspring delivered by the female host for the presence of the modification.

For this method of producing a non-human vertebrate or mammal, fertilisation of the oocyte is required. Said fertilisation can occur before the modification of the target sequence in step (a) in accordance with the method of producing a non-human vertebrate or mammal of the invention, i.e. a fertilised oocyte can be used for the method of modifying a target sequence in accordance with the invention. The fertilisation can also be carried out after the modification of the target sequence in step (a), i.e. a non-fertilised oocyte can be used for the method of modifying a target sequence in accordance with the invention, wherein the oocyte is subsequently fertilised before transfer into the pseudopregnant female host.

The step of analysing for the presence of the modification in the offspring delivered by the female host provides the necessary information whether or not the produced non-human vertebrate or mammal carries the modified target sequence in its genome. Thus, the presence of the modification is indicative of said offspring carrying a modified target sequence in its genome whereas the absence of the modification is indicative of said offspring not carrying the modified target sequence in its genome. Methods for analysing for the presence or absence of a modification have been detailed above.

The non-human vertebrate or mammal produced by the method of the invention is, inter alia, useful to study the function of genes of interest and the phenotypic expression/outcome of modifications of the genome in such animals. It is furthermore envisaged, that the non-human mammals of the invention can be employed as disease models and for testing therapeutic agents/compositions. Furthermore, the non-human vertebrate or mammal of the invention can also be used for livestock breeding.

In a preferred embodiment, the method of producing a non-human vertebrate or mammal further comprises culturing the cell to form a pre-implantation embryo or introducing the cell into a blastocyst prior to transferring it into the pseudo pregnant female host. Methods for culturing the cell to form a pre-implantation embryo or introducing the cell into a blastocyst are well known in the art and are, for example, described in Nagy et al., loc. cit.

The term “introducing the cell into a blastocyst” as used herein encompasses injection of the cell into a blastocyst as well as fusion of a cell with a blastocyst. Methods of introducing a cell into a blastocyst are described in the art, for example in Nagy et al., loc. cit.

The present invention further relates to a non-human vertebrate or mammalian animal obtainable by the above described method of the invention.

In a preferred embodiment of the methods of the invention, the cell is from a mammal selected from the group consisting of rodents, dogs, felides, primates, rabbits, pigs, or cows or the cell is from an avian selected from the group consisting of chickens, turkeys, pheasants, ducks, geese, quails and ratites including ostriches, emus and cassowaries or the cell is from a fish such as for example a zebrafish, salmon, trout, common carp or coi carp.

All of the mammals, avians and fish described herein are well known to the skilled person and are taxonomically defined in accordance with the prior art and the common general knowledge of the skilled person.

Non-limiting examples of “rodents” are mice, rats, squirrels, chipmunks, gophers, porcupines, beavers, hamsters, gerbils, guinea pigs, degus, chinchillas, prairie dogs, and groundhogs.

Non-limiting examples of “dogs” include members of the subspecies Canis lupus familiaris as well as wolves, foxes, jackals, and coyotes.

Non-limiting examples of “felides” include members of the two subfamilies: the pantherinae, including lions, tigers, jaguars and leopards and the felinae, including cougars, cheetahs, servals, lynxes, caracals, ocelots and domestic cats.

The term “primates”, as used herein, refers to all monkey including for example cercopithecoid (old world monkey) or platyrrhine (new world monkey) as well as lemurs, tarsiers, apes and marmosets (Callithrix jacchus).

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show:

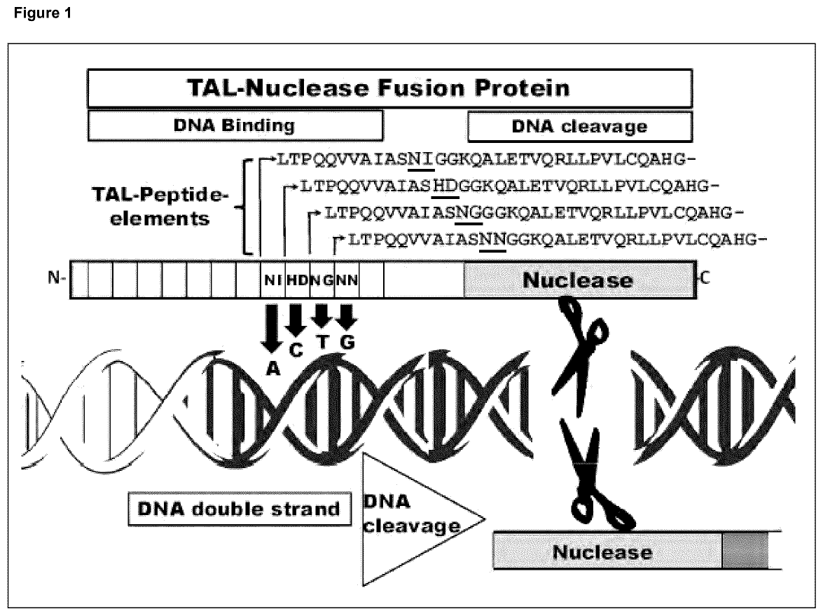

FIG. 1: TAL-Nuclease expression vectors.

The figure shows the structure and function of TAL-Nuclease fusion proteins, consisting of a sequence-specific DNA-binding domain and a nonspecific DNA cleavage (nuclease) domain. The DNA-binding domain can be assembled from the four types of 34 amino acid TAL peptide elements that exhibit binding specificity against one of the DNA nucleotides through the amino acid positions 12 and 13 (NI-A; HD-C; NG-T; NN-G). Upon binding of the TAL element domain to the selected target DNA sequence, the nuclease domain of the fusion protein comes into close contact to the DNA double-strand but does not cleave the DNA as a nuclease monomer. Only upon the binding of a second TAL-Nuclease fusion protein to a second DNA target sequence located downstream of the binding site of the first fusion protein, the DNA double strand is cleaved through cooperation of the two nuclease domains that are in close contact.

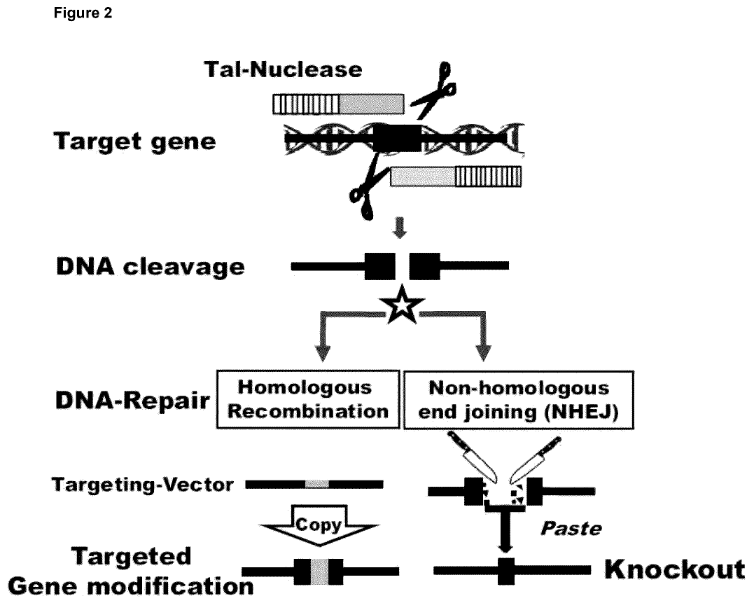

FIG. 2: TAL-Nuclease induced modification of genomic sequences.

The figure shows a pair of TAL-nuclease fusion proteins that bind up- and downstream of a selected target site within a genomic target gene. Upon the creation of a DNA double-strand break within the target site two competing DNA repair mechanisms are strongly activated in cells: i) by homologous recombination, in the presence of an externally introduced gene targeting vector that comprises two homology regions to the target gene and a predesigned genetic modification/mutation, the preplanned modification is copied from the targeting vector into the genome; by this route any targeted gene modification (e.g. knock-out, knock-in) can be placed into the genome, ii) by the non-homologous end joining repair pathway (NHEJ) the free DNA ends are closed by ligation without a repair template; by this route a variable number of nucleotides is frequently lost (knife symbol) before end ligation and results frequently into a knockout allele of the target gene.

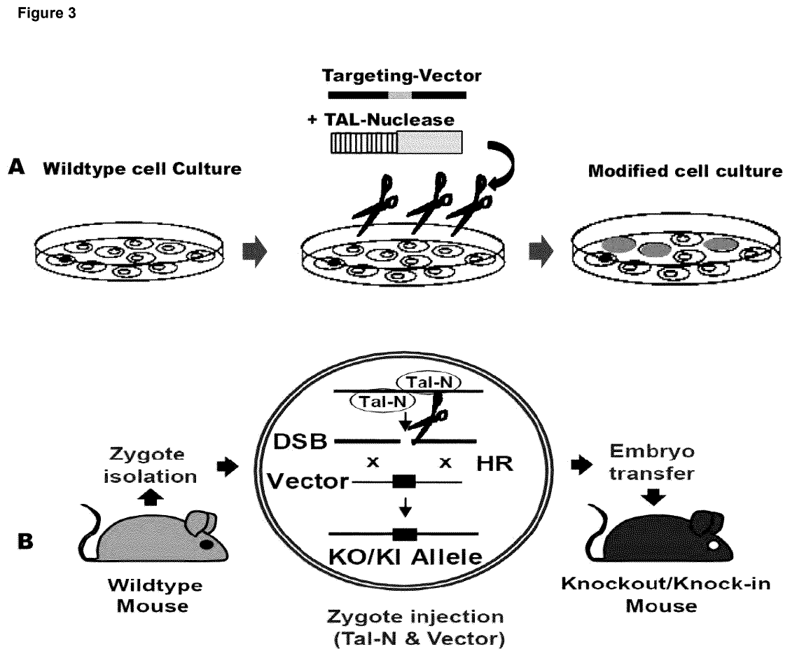

FIG. 3: Use of TAL-Nucleases for gene targeting in mammalian cell lines and zygotes.

A: For the generation of genetic modifications in mammalian cell lines TAL-nuclease expression vectors can be transfected, together with or without a specific gene targeting vector, into cultured cells. Upon nuclease expression and DNA repair a fraction of the treated cells contains the desired genetic alteration. These cells can be isolated and further cultured as a pure genetically modified cell line. B: Upon the microinjection of TAL-nuclease mRNA, together with or without a specific gene targeting vector, into fertilized mammalian oocytes (zygotes, isolated from wildtype female e.g. mice) a knockout (KO) or Knockin (KI) allele can be directly introduced into the genome of the one-cell embryo. Pseudopregnant females deliver live offspring from microinjected oocytes. The offspring is genotyped for the presence of the induced genetic modification. Positive animals are selected for further breeding to establish a gene targeted strain.

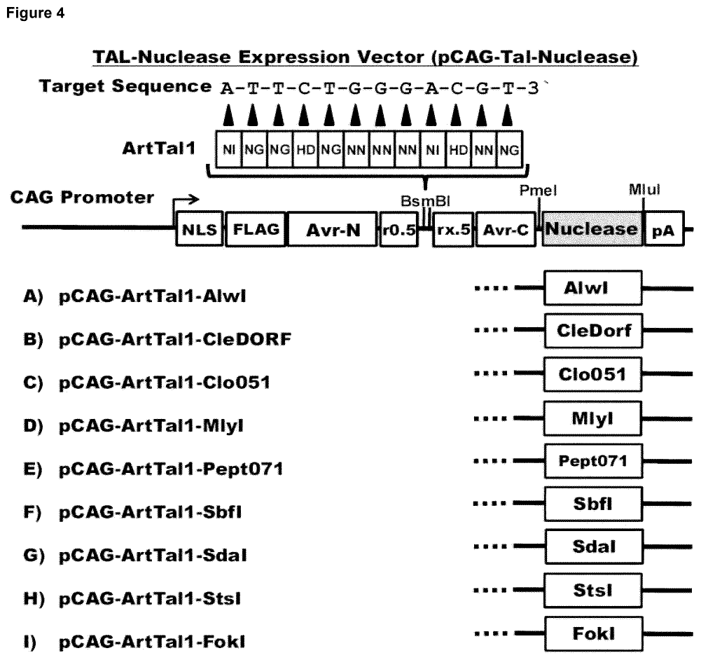

FIG. 4: TAL-Nuclease expression vectors.

The Tal nuclease expression vector pCAG-Tal-nuclease contains a CAG promoter region and a transcriptional unit comprising, upstream of a central pair of BsmBI restriction sites, an ATG start codon (arrow), a nuclear localisation sequence (NLS), a FLAG Tag sequence (FLAG), a linker sequence, a segment coding for 110 amino acids of the Tal protein AvrBs3 (AvrN) and its invariable N-terminal Tal repeat (r0.5). Downstream of the BsmBl sites the transcriptional unit contains an invariable C-terminal Tal repeat (rx.5), a segment coding for 44 amino acids derived from the Tal protein AvrBs3, a PmeI and MluI restriction site for the insertion of nuclease coding regions and a polyadenylation signal sequence (pA). DNA segments coding for TAL repeat elements can be inserted into the BsmBI sites of pCAG-Tal-nuclease for the expression of variable TAL-nuclease fusion proteins. To create ArtTal1-nuclease expression vectors the ArtTal1 array of TAL repeat elements, recognizing the specified 12 bp target sequence, was inserted into the BsmBl sites of pCAG-TAL-nuclease. Each 34 amino acid Tal repeat is drawn as a square indicating the repeat's amino acid code at positions 12/13 that confers binding to one of the DNA nucleotides of the target sequence (NI>A, NG>T, HD>C, NN>G) shown above. Next, synthetic nuclease domain coding regions were inserted into the PmeI and MluI sites of pCAG-ArtTal1-nuclease to obtain the expression vectors: A: pCAG-ArtTal1-Alw including the nuclease domain of the AlwI restriction endonuclease, B: pCAG-ArtTal1-CleDORF including the nuclease domain of the CIeDORF gene, C: pCAG-ArtTal1-Clo051 including the nuclease domain of the Clo051 gene, D: pCAG-ArtTal1-Mly including the nuclease domain of the MlyI restriction endonuclease, E: pCAG-ArtTal1-Pept071 including the nuclease domain of the Pept071 gene, F: pCAG-ArtTal1-Sbf including the nuclease domain of the SbfI restriction endonuclease, G: pCAG-ArtTal1-SdaI including the nuclease domain of the SdaI restriction endonuclease, H: pCAG-ArtTal1-Sst including the nuclease domain of the StsI restriction endonuclease, and I: pCAG-ArtTal1-Fok including the nuclease domain of the FokI restriction endonuclease

FIG. 5: Amino acid sequence of the Clo051 protein

Sequence of the 587 amino acid Clo051 protein in the single letter code. Indicated are the methionine at position 1 (M1), the tyrosine at position 587 (Y587) and the 199 residue nuclease domain between position E389 and Y587. Further highlighted are the positions D455, D472 and K474 that are characteristic for the conserved active site of the ‘PD-(D/E)XK’ superfamily of enzymes interacting with DNA.

FIG. 6: Predicted structure of the Clo051 protein and its Nuclease Domain.

The tertiary structure of the Clo051 protein was predicted from its amino acid sequence (FIG. 5) using the I-TASSER software. The secondary structures are shown as alpha-helical and beta-stranded regions. Highlighted are the methionine at position 1 (M1), the glutamate residue 389 (E389) and tyrosine 587 (Y587). The protein chain between E389 and Y587 forms a separate folding domain that acts as a nuclease.

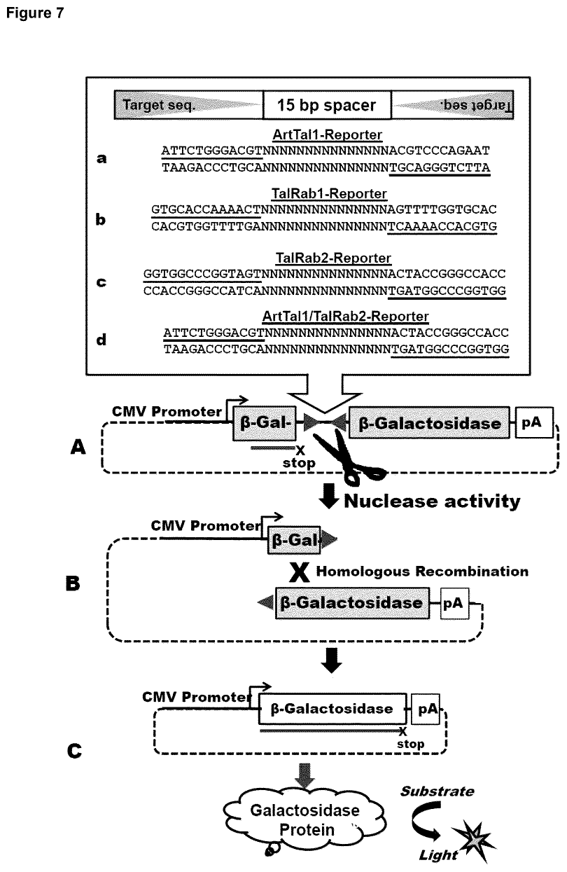

FIG. 7: TAL-Nuclease reporter plasmids and nuclease reporter assay.

A: TAL-nuclease reporter plasmids contain a CMV promoter region, a 400 bp sequence coding for the N-terminal segment of 0-galactosidase and a stop codon. This unit is followed by a TAL binding target region consisting of two inverse oriented recognition sequences (underlined), separated by a 15 bp spacer region (NNN.), for the ArtTal1 array (a), the TalRab1 array (b), the TalRab2 array (c), or a hybrid binding region composed of one ArtTal1 and one TalRab2 recognition sequence (d). The TAL-nuclease target region is followed by the complete coding region for β-galactosidase and a polyadenylation signal (pA). To test for nuclease activity against the target sequence a TAL-nuclease expression vector (FIG. 4) is transiently cotransfected with its corresponding reporter plasmid into HEK 293 cells. Upon expression of the TAL-nuclease protein the reporter plasmid is opened by a nuclease-induced double-strand break within the TAL-nuclease target sequence (scissor symbol). B: The DNA regions adjacent to the double-strand break are identical over 400 bp and can be aligned and recombined (X) by homologous recombination DNA repair. C: Homologous recombination of an opened reporter plasmid results into a functional β-galactosidase expression vector that produces the β-galactosidase enzyme. After two days the transfected cells are lysed and the enzyme activity in the lysate is determined with a chemiluminescent reporter assay. The levels of the reporter catalysed light emission are measured and indicate TAL-nuclease activity in comparison to samples that were transfected with the reporter plasmid alone.

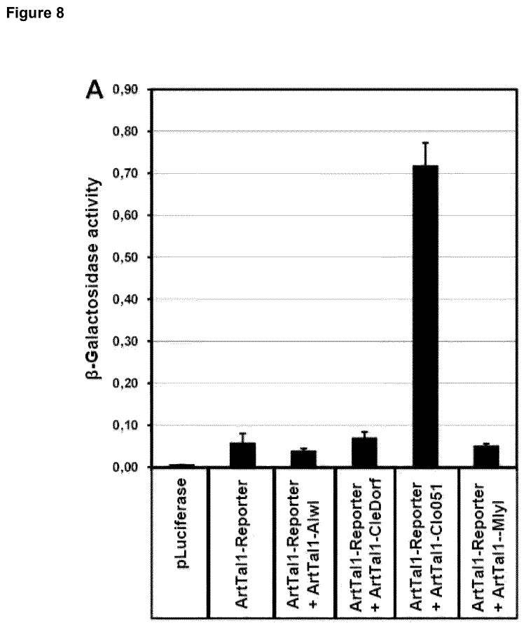

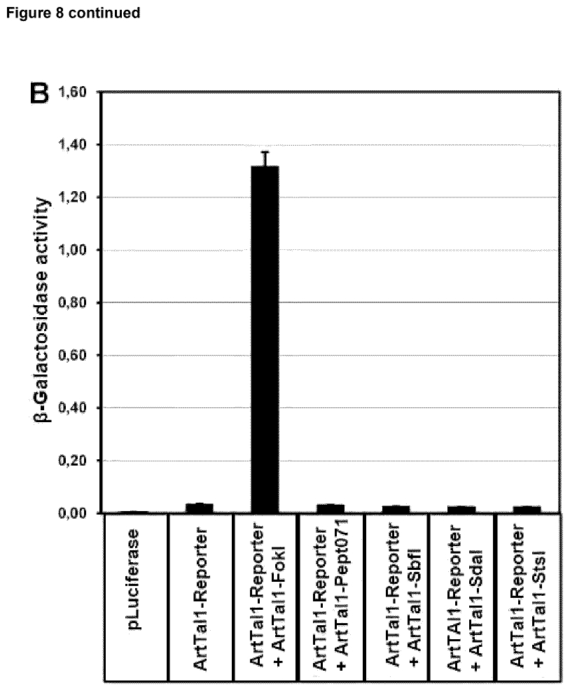

FIG. 8: Activity of Tal nuclease fusion proteins in HEK 293 cells.

To test for the nuclease activity of TAL-nuclease domain fusion proteins, expression vectors for the ArtTal1-AlwI, -CIeDORF, -Clo051, -MlyI, -FokI, -Pept071, -SbfI, -SdaI, and -StsI proteins (FIG. 4) were transfected together with the ArtTal1 reporter plasmid (FIG. 7) into HEK 293 cells. Specific nuclease activity against the reporter plasmid's target sequence leads to homologous recombination and the expression of 0-galactosidase. Two days after transfection the cell populations were lysed and the 0-galactosidase activity determined with a chemiluminescent reporter assay. The levels of light emission were normalised in relation to the activity of a cotransfected Luciferase expression plasmid (pLuciferase) and are shown in comparison to the activity of a positive control β-galactosidase expression vector. The bar for each transfected sample represents the mean value and SD derived from three culture wells transfected side by side. A: The transfection of the ArtTal1 reporter plasmid without nuclease expression vector results in a low background level of p-galactosidase. The cotransfection of pCAG-ArtTal1-AlwI, -CIeDORF, and -MlyI with the ArtTal1 reporter plasmid did not lead to a significant increase of reporter expression, indicating that the ArtTal1-AlwI, -CIeDORF, and -MlyI fusion proteins do not exhibit nuclease activity. In contrast, the cotransfection of the ArtTal1 reporter and the pCAG-ArtTal1-Clo051 plasmids resulted in a strong increase of reporter expression, indicating that the ArtTal1-Clo051 fusion protein exhibits target specific nuclease activity in 293 cells. B: In an independent transfection experiment the cotransfection of pCAG-ArtTal1-Pept071, -SbfI, -SdaI and -Sst with the ArtTal1 reporter plasmid did not lead to a significant increase of reporter expression, as compared to the ArtTal1 reporter plasmid alone, indicating that the ArtTal1-Pept071, -SbfI, -SdaI, and -StsI fusion proteins do not exhibit nuclease activity. In contrast, the cotransfection of the ArtTal1 reporter and the pCAG-ArtTal1-FokI plasmids resulted in the increase of reporter expression, indicating the nuclease activity of the ArtTal1-FokI fusion protein in 293 cells.

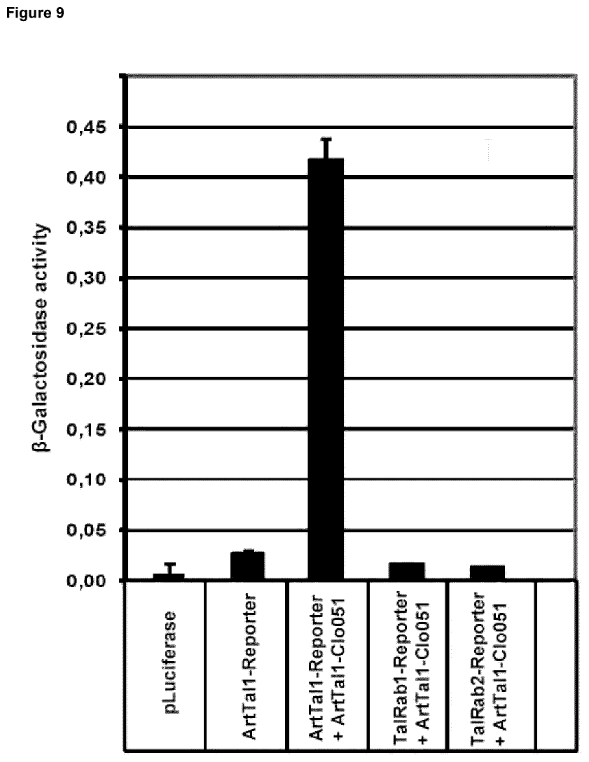

FIG. 9: Target sequence specificity of the ArtTal1-Clo051 nuclease.

To test for the specificity of the ArtTal1-Clo051 nuclease against the predesigned target sequence in comparison to unrelated DNA sequences, the pCAG-ArtTal1-Clo051 expression vector was cotransfected with the corresponding ArtTal1-reporter plasmid or with the TalRab1 or TalRab2 reporter plasmids (FIG. 7), which contain unrelated target sequences, into HEK 293 cells. Strong nuclease activity developed only in the specific combination of the ArtTal1-Clo051 expression vector together with the ArtTal1-reporter plasmid, indicating that the ArtTal1-Clo051 nuclease acts specifically against the predesigned target sequence.

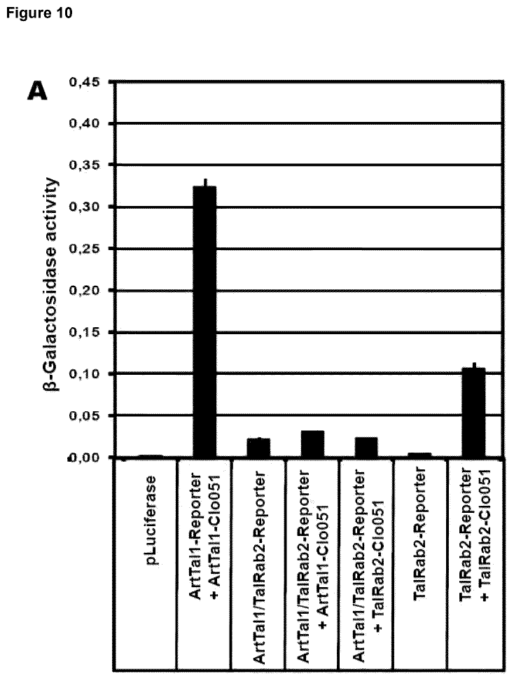

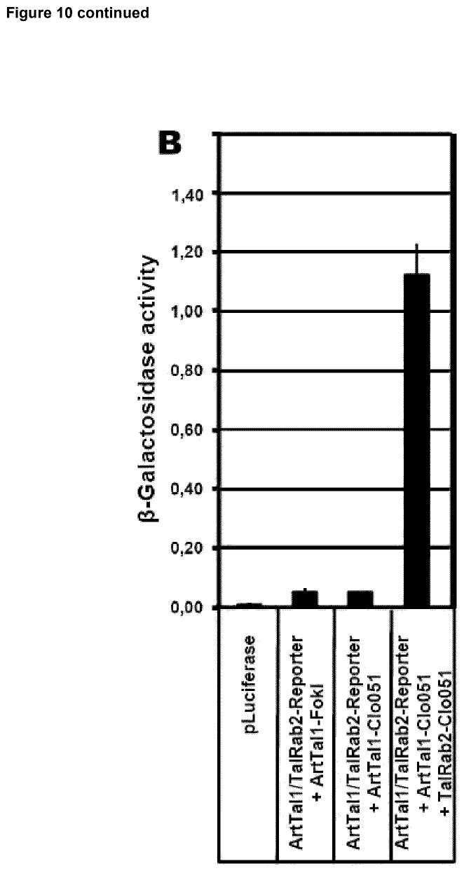

FIG. 10: Characterisation of the Cooperativity of TAL-Clo051 nuclease fusion proteins

A: To test for the cooperativity of the Clo051 nuclease domains of a pair of TAL-Clo051 fusion proteins, expression vectors for the ArtTal1-Clo051 or TalRab2-Clo051 fusion proteins were cotransfected with the corresponding ArtTal1- or TalRab2-reporter plasmid (FIG. 7) and compared to the cotransfection with the ArtTal1/TalRab2-reporter plasmid, that contains a hybrid target region (FIG. 7). Significant nuclease activity developed only in the combination of TAL-nuclease expression vectors with reporter plasmids that contain two identical, inverse copies of the corresponding TAL array target sequence, but not with the ArtTal1/TalRab2-reporter plasmid that contains only a single binding sequence of the ArtTal1-Clo051 and TalRab2-Clo051 fusion proteins. This result indicates that two Clo051 nuclease domains must cooperate to induce a DNA double-strand break, whereas a single Clo051 nuclease domain does not act as a nuclease. B: The cotransfection of the ArtTal1/TalRab2-reporter plasmid with both expression vectors for ArtTal1-Clo051 and TalRab2-Clo051, but not with ArtTal1-Clo051 or -Fok alone, results into strong nuclease activity, as compared to the transfection of the ArtTal1/TalRab2 reporter plasmid. This result indicates that nuclease activity and the induction of double-strand breaks in the target region occurs only upon the binding of two TAL-Clo051 fusion proteins and the interaction of a pair of Clo051 nuclease domains.

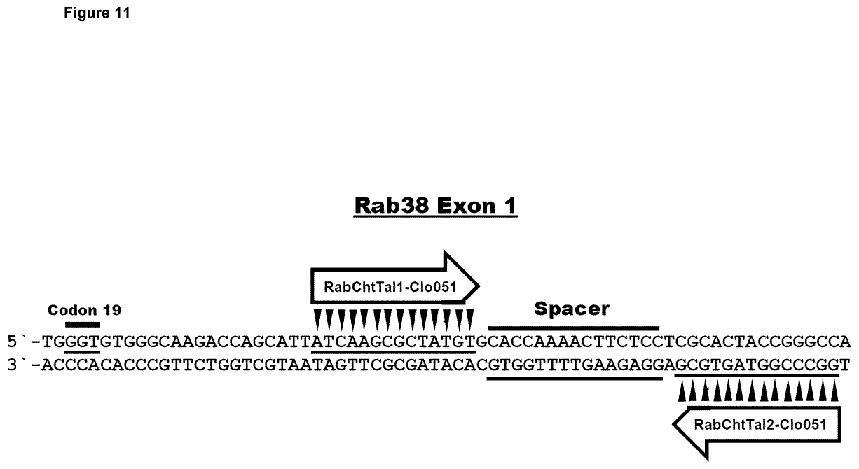

FIG. 11: Design of a TAL-Clo051 Fusion Protein Pair in Accordance with the Present Invention, Recognizing the Mouse Rab38 Gene.

TAL nucleases recognizing a target sequence within exon 1 of the mouse Rab38 gene. The trinucleotide representing codon 19 is underlined. Indicated is each of a 14 nucleotide sequence that is recognised by one the indicated TAL-Clo051 fusion proteins RabChtTal1- and RabChtTa12-Clo051. The two 14 bp target sequences are flanking a central 15 bp spacer sequence that is cleaved by the Clo051 nuclease domains.

FIG. 12: Strategy for the Modification of the Mouse Rab38 Gene in ES Cells and Zygotes Using TAL-Clo051 Fusion Proteins.

Within exon 1 of the wildtype Rab38 gene (Rab38 WT) the position of the binding sites for the TAL nuclease pair RabChtTal1- and RabChtTa12-Clo051 are indicated. The Rab38-cht targeting vector contains a 942 bp 5′-homology region and a 2788 bp 3′-homology region flanking the Rab38 TAL recognition sites. Within exon1 two nucleotide changes within codon 19 (Gta) of Rab38 create a chocolate (cht) missense mutation coding for valine (Val) instead of the wildtype (WT) glycine (Gly), and remove a BsaJI restriction site. In each of the adjacent Rab38 TAL recognition sites several silent mutations were introduced to prevent the binding of Rab38 TAL proteins to the targeting vector. The induction of a double-strand break within the wildtype Rab38 gene by the RabChtTa1 protein pair stimulates homologous recombination with the Rab38-cht targeting vector and integrates the chocolate missense and the silent mutations into the genome.

FIG. 13: Isolation of Hyperactive Clo051 Nuclease Mutants.

The figure shows the primary sequence of the Clo051 nuclease domain between the positions E389 and Y587. Indicated is the distribution of the positively charged arginine (R) and lysine (K) residues (filled squares) and of negatively charged glutamate (E) and aspartate (D) residues (open circles). Triangles indicate the positions S423 and R446. These residues constitute a three-dimensional framework of charges within the Clo051 domain that determines the unique tertiary structure of this nuclease, as modelled in the structure of FIG. 6. Certain replacements of polar versus non-polar residues or of non-polar residues against polar residues, e.g. at the positions 423 and 446, changes the three-dimensional structure of the protein chain and results into a more efficiently working nuclease activity.

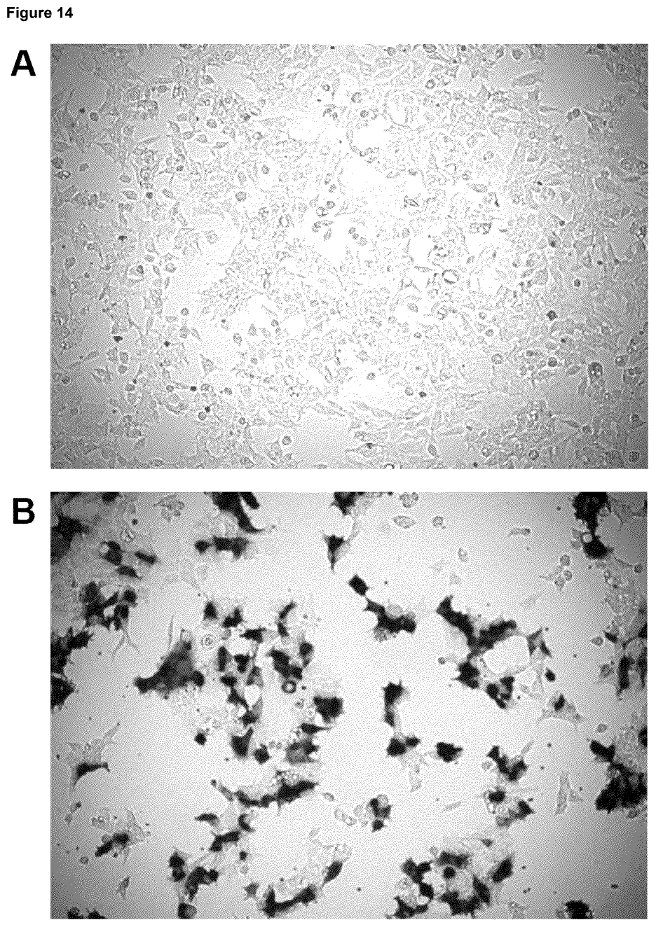

FIG. 14: Activity of ArtTal1-Clo051 nuclease on a genomic reporter in HEK 293 Cells

HEK293 cells harboring genomic integrated copies of the pCMV-Rab-Reporter(hygro) reporter construct were transfected with pBluescript or pCAG.ArtTal1-Clo051. Specific nuclease activity against the reporter's target sequence leads to homologous recombination and the expression of β-galactosidase. Two days after transfection the cell populations were fixed and the fraction of β-galactosidase expressing cells was determined by histochemical X-Gal staining. A: X-Gal stained reporter cell culture upon transfection with pBluescript. B: X-Gal stained reporter cell culture upon transfection with pCAG-ArtTal1-Clo051 nuclease expression vector.

The examples illustrate the invention:

### EXAMPLE 1

### Construction of Expression and Reporter Vectors for Tal Nucleases and Detection of Specific Nuclease Activity

### Construction of TAL-Nuclease Expression Vectors

For the expression of TAL-nucleases in mammalian cells we designed the generic expression vector pCAG-TAL-nuclease (SEQ ID NO: 3) (FIG. 4), that contains a CAG hybrid promoter region and a transcriptional unit comprising a sequence coding for a N-terminal peptide of 176 amino acids (SEQ ID NO: 4) of TAL nuclease fusion proteins, located upstream of a pair of BsmBI restriction sites. This N-terminal regions includes an ATG start codon, a nuclear localisation sequence, a FLAG Tag sequence, a glycine rich linker sequence, a segment coding for 110 amino acids of the Tal protein AvrBs3 and the invariable N-terminal Tal repeat of the Hax3 TAL effector. Downstream of the central BsmBl sites, the transcriptional unit contains 78 codons (SEQ ID NO: 5) including an invariable C-terminal TAL repeat (34 amino acids) and 44 residues derived from the TAL protein AvrBs3, followed by a PmeI and MluI restriction site for the insertion of a nuclease coding region and by a polyadenylation signal sequence (pA). DNA segments coding for arrays of TAL repeats, designed to bind a TAL nuclease target sequence can be inserted into the BsmBI sites of pCAG-Tal-nuclease in frame with the up- and downstream coding regions for the expression of predesigned TAL-nuclease proteins.

To generate TAL-nuclease vectors for expression in mammalian cells we inserted a synthetic DNA segment with the coding region of an array of 12 Tal repeats, designated ArtTal1 (SEQ ID NO: 6), into the BsmBI sites of pCAG-TAL-nuclease, to derive the plasmid pCAG-ArtTal1-nuclease (SEQ ID NO: 7). The TAL element array ArtTal1 recognises the artificial DNA target sequence 5′-ATTCTGGGACGT-3′ (SEQ ID NO: 62) (FIG. 4), In another example we inserted a synthetic DNA segment with the coding region of an array of 14 Tal repeats, designated TalRab2 (SEQ ID NO: 8), into the BsmBI sites of pCAG-TAL-nuclease, to derive the plasmid pCAG-TalRab2-nuclease (SEQ ID NO: 9). The TAL element array TalRab2 recognises the DNA target sequence 5′-GGTGGCCCGGTAGT-3′ (SEQ ID NO: 63) (FIG. 7) that occurs within the mouse Rab38 gene. The TAL target sequences were selected such that the binding regions of the TAL proteins are preceded by a T nucleotide. Following the sequence downstream of the initial T in the 5>3′ direction, specific TAL DNA-binding domains were combined together into arrays of 12 (ArtTal1) (FIG. 4), or 14 (TalRab2) TAL elements. Each TAL element motif consists of 34 amino acids, the position 12 and 13 of which determines the specificity towards recognition of A, G, C or T within the target sequence. To derive TAL element DNA-binding domains we used the TAL effector motif (repeat) #11 of the Xanthomonas Hax3 protein (GenBank accession No. AY993938.1 (LTPEQVVAIASNIGGKQALETVQRLLPVLCQAHG) (SEQ ID NO: 64) with amino acids N12 and 113 to recognize A, the TAL effector motif (repeat) #5 (LTPQQVVAIASHDGGKQALETVQRLLPVLCQAHG) (SEQ ID NO: 65) derived from the Hax3 protein with amino acids H12 and D13 to recognize C, and the TAL effector motif (repeat) #4 (LTPQQVVAIASNGGGKQALETVQRLLPVLCQAHG) (SEQ ID NO: 66) from the Xanthomonas Hax4 protein (Genbank accession No.: AY993939.1) with amino acids N12 and G13 to recognize T. To recognize a target G nucleotide we used the TAL effector motif (repeat) #4 from the Hax4 protein with replacement of the amino acids 12 into N and 13 into N (LTPQQVVAIASNNGGKQALETVQRLLPVLCQAHG) (SEQ ID NO: 67).

Next, we constructed fusion proteins of the ArtTal1 DNA binding domain with protein domains derived from known or putative nucleases and tested whether these TAL-nuclease fusion proteins are able to induce a double-strand break next to the DNA bound by the TAL recognition region. For this purpose we inserted synthetic DNA segments comprising the coding regions of eight putative nuclease domains and the known nuclease domain of FokI (SEQ ID NO: 10), into the PmeI and MluI sites of the pCAG-ArtTal1-nuclease plasmid. Among the eight putative nuclease domains we selected domains from the five known restriction enzymes AlwI (SEQ ID NO: 11), MlyI (SEQ ID NO: 12), SbfI (SEQ ID NO: 13), SdaI (SEQ ID NO: 14) and StsI (SEQ ID NO: 15). In addition, we selected putative nuclease domains of three yet uncharacterised, hypothetical microbial genes, designated here as ‘CIeDORF’ (SEQ ID NO: 16) (NCBI Reference Sequence: ZP_02080987.1, derived from the genome of Clostridium leptum DSM753), ‘Clo051 (SEQ ID NO: 17) (NCBI Reference Sequence: ZP_05132802.1, derived from the genome of Clostridium spec. 7_2_43FAA) and ‘Pept071’ (SEQ ID NO: 18) (NCBI Reference Sequence: ZP_07399918.1, derived from the genome of Peptoniphilus duerdenii ATCC BAA-1640). These proteins were selected by characteristic sequence features that are compatible with the conserved active site of the ‘PD-(D/E)XK’ superfamily of enzymes (Kosinski, J., et al. (2005). BMC Bioinformatics, 6,172) interacting with DNA (see FIG. 6 for the Clo051 protein).

In particular, the 587 residue Clo051 protein can be classified as a member of the PD-(D/E)XK protein family by the location of the amino acid pairs P454/D455 (PD motif) and D472/K474 (DXK motif) (FIG. 5). To elucidate whether the Clo051 protein contains a separate nuclease domain we performed a three-dimensional structural prediction from its primary amino acid sequence using the I-TASSER software (Roy, A. et al. (2010). Nat Protoc., 5(4):725-38). As shown in FIG. 6 the Clo051 protein is composed of two protein domains. The C-terminal domain of Clo051, approximately beginning with the residue E389, contains the PD-(D/E)XK family consensus motif and appears as a non specific nuclease domain.

For the expression of these protein domains in mammalian cells we used synthetic coding regions optimised according to the mammalian codon usage and inserted segments comprising the putative nuclease domains of AlwI (SEQ ID NO: 19), CleDORF (SEQ ID NO: 20), Clo051 (SEQ ID NO: 1), MlyI (SEQ ID NO: 21), Pept071 (SEQ ID NO: 22), SbfI (SEQ ID NO: 23), SdaI (SEQ ID NO: 24), StsI (SEQ ID NO: 25) and the known nuclease domain of FokI (SEQ ID NO: 26) into the PmeI and MluI sites of the pCAG-ArtTal1-nuclease plasmid, to derive the expression vectors pCAG-ArtTal1-AlwI (SEQ ID NO: 27) (FIG. 4A), pCAG-ArtTal1-CleDORF (SEQ ID NO: 28) (FIG. 4B), pCAG-ArtTal1-Clo051 (SEQ ID NO: 29) (FIG. 4C), pCAG-ArtTal1-Mly1 (SEQ ID NO: 30) (FIG. 4D), pCAG-ArtTal1-Pept071 (SEQ ID NO: 31) (FIG. 4E), pCAG-ArtTal1-Sbf1 (SEQ ID NO: 32) (FIG. 4F), pCAG-ArtTal1-SdaI (SEQ ID NO: 33) (FIG. 4G), pCAG-ArtTal1-StsI (SEQ ID NO: 34) (FIG. 4H), and pCAG-ArtTal1-FokI (SEQ ID NO: 35) (FIG. 4I). These expression vectors code for the TAL-fusion proteins designated as ArtTal1-AlwI (SEQ ID NO: 36), ArtTal1-CleDORF (SEQ ID NO: 37), ArtTal1-Clo051 (SEQ ID NO: 38), ArtTal1-MlyI (SEQ ID NO: 39), ArtTal1-Pept071 (SEQ ID NO: 40), ArtTal1-Sbf1 (SEQ ID NO: 41), ArtTal1-SdaI (SEQ ID NO: 42), ArtTal1-StsI (SEQ ID NO: 43), and ArtTal1-FokI (SEQ ID NO: 44).

### Construction of TAL Nuclease Reporter Plasmids

To determine the activity and specificity of TAL nuclease domain fusion proteins in mammalian cells we constructed TAL nuclease reporter plasmids that contain two copies of a TAL DNA target sequence in inverse orientation, separated by a 15 nucleotide spacer region (FIG. 7a-d). This configuration enables to measure the activity of a single type of TAL nuclease that interacts as a homodimer of two protein molecules that are bound to the inverse pair of target sequences of the reporter plasmid. Upon DNA binding and interaction of two nuclease domains the reporter plasmid DNA is cleaved within the 15 bp spacer region and exhibits a double-strand break.

The TAL nuclease reporter plasmids contain a CMV promoter region, a 400 bp sequence coding for the N-terminal segment of β-galactosidase and a stop codon. This unit is followed by the TAL nuclease target region (consisting of two inverse oriented recognition sequences separated by a 15 bp spacer region) for ArtTal1-fusion proteins in the plasmid ArtTal1-reporter (SEQ ID NO: 45)(FIG. 7a), by the unrelated target sequence TalRab1 in the TalRab1-reporter plasmid (SEQ ID NO: 46) (FIG. 7b), by the target region for TalRab2 fusion proteins in the TalRab2-reporter plasmid (SEQ ID NO: 47) (FIG. 8c), or a hybrid target region containing one copy of the ArtTal1 and the TalRab2 recognition sequence in the ArtTal1/TalRab2-reporter plasmid (SEQ ID NO: 48) (FIG. 8d).

Within these reporter plasmids the TAL nuclease target regions are followed by the complete coding region for β-galactosidase and a polyadenylation signal (pA). To test for nuclease activity against the specific target sequence a TAL nuclease expression vector (FIG. 4) was transiently cotransfected with its corresponding reporter plasmid into mammalian cells. Upon expression of the TAL nuclease protein the reporter plasmid is opened by a nuclease-induced double-strand break within the TAL nuclease target sequence (FIG. 7A). The DNA regions adjacent to the double-strand break are identical over 400 bp and can be aligned and recombined by homologous recombination DNA repair (FIG. 7B). Homologous recombination of an opened reporter plasmid will subsequently result into a functional β-galactosidase coding region transcribed from the CMV promoter that leads to the production of β-galactosidase protein (FIG. 7C). In lysates of transfected cells the enzymatic activity of β-galactosidase can be determined by chemiluminescence and reports the nuclease activity of the TAL fusion proteins.

### Measurement of TAL-Nuclease Activity and Specificity in Human 293 Cells

To determine the activity and specificity of TAL nucleases in mammalian cells, we electroporated one million HEK 293 cells (ATCC #CRL-1573) (Graham F L, Smiley J, Russell W C, Nairn R., J. Gen. Virol. 36, 59-74, 1977) with 5 μg plasmid DNA of one of the TAL nuclease expression vectors (FIG. 4) together with 5 μg of one of the TAL nuclease reporter plasmids (FIG. 7). In addition, each sample received 5 μg of the firefly Luciferase expression plasmid pCMV-hLuc (SEQ ID NO: 49) and was adjusted to a total DNA amount of 20 μg with pBluescript (pBS) plasmid DNA (SEQ ID NO: 50). Upon transfection the cells were seeded in triplicate wells of a 6-well tissue culture plate and cultured for two days before analysis was started. For analysis the transfected cells of each well were lysed and the β-galactosidase and luciferase enzyme activities of the lysates were individually determined using chemiluminescent reporter assays following the manufacturer's instruction (Roche Applied Science, Germany) in a luminometer (Berthold Centro LB 960). As positive control we transfected 5 μg of the β-galactosidase expression plasmid pCMVβ (SEQ ID NO: 51) with 15 μg pBS, as negative control 5 μg pCMV-hLuc were transfected with 15 μg pBS or 5 μg pCMV-hLuc together with 5 μg of a TAL nuclease reporter plasmid and 10 μg pBS. The triplicate β-galactosidase values of each sample were normalised in relation to the levels of Luciferase activity and the mean value and standard deviation of β-galactosidase activity were calculated and expressed in comparison to the pCMVp positive control. In this type of recombination assay the level of the p-galactosidase catalysed light emission reflects the cleavage and repair of the reporter plasmids and thereby indicates the activity of TAL nucleases.

As shown in FIG. 8 transfection of the ArtTal1-Reporter plasmid alone resulted in just background levels of p-galactosidase. The cotransfection of the ArtTal1-Reporter plasmid with the expression vectors pCAG-ArtTal1-AlwI, -CleDORF, -MlyI, -Pept071, -SbfI, -SdaI, and -StsI did not reveal any significant nuclease activity of the encoded TAL fusion proteins (FIG. 8), indicating that the selected nuclease domains are unable to operate in combination with TAL DNA binding elements. In contrast, the cotransfection of the ArtTal1-Reporter plasmid with the expression vectors pCAG-ArtTal1-Clo051 (FIG. 8A) and -FokI (FIG. 8b) resulted in significantly increased reporter activity, indicating that the selected FokI and Clo051 protein domains are able to function as nuclease in fusion with TAL DNA binding elements.

Since in repeated assays TAL fusions with the Clo051 domain appeared more active as compared to fusions with the FokI nuclease domain, we believe that the Clo051 domain is most suited for the construction of highly active TAL-nucleases.

In order to define whether the ArtTal1-Clo051 nuclease specifically recognizes its target sequence within the ArtTal1-reporter plasmid (FIG. 7a), pCAG-ArtTal1-Clo051 was cotransfected with the corresponding ArtTal1- or with the unrelated TalRab1- or TalRab2 reporter plasmids (FIG. 7b,c) into HEK 293 cells. As shown in FIG. 9 significantly increased reporter activity was detected only from the specific combination of the ArtTal-Clo051 nuclease with its corresponding promoter, whereas the cotransfection with unrelated reporter plasmids did not exhibit significant nuclease activity. These results indicate that the Clo051 nuclease domain in fusion with TAL DNA binding elements acts in a target sequence specific manner and that unrelated target sequences are not processed.

Next, we characterized whether the Clo051 nuclease domain induces recombinogenic double-strand breaks as a monomer, or whether the interaction of two nuclease domains as dimer is required. For this purpose we constructed the hybrid reporter plasmid ArtTal1/TalRab2-reporter (SEQ ID NO: 48) (FIG. 7d) that contains one ArtTal1 recognition sequence upstream of the spacer region and one TalRab2 recognition sequence downstream of the spacer region. The TalRab2 array (SEQ ID NO: 8) is composed of 14 TAL elements recognising the target sequence 5′-GGTGGCCCGGTAGT-3′ (SEQ ID NO: 63). The Clo051 nuclease domain was cloned as synthetic coding region into the PmeI and MluI sites of plasmid pCAG-TalRab2-nuclease (SEQ ID NO: 9) to derive the expression vector pCAG-TalRab2-Clo051 (SEQ ID NO: 52) for the expression of the TalRab2-Clo051 protein (SEQ ID NO: 53). As shown in FIG. 10A the cotransfection of pCAG-ArtTal1-Clo051 together with the ArtTal1-reporter plasmid resulted in significant reporter gene expression indicating specific nuclease activity of the ArtTal1-Clo051 fusion protein. Since the ArtTal1-reporter plasmids contains two inverse ArtTal1 binding sequences, the nuclease activity of ArtTal1-Clo051 may result from the action of a single fusion protein or the combined action of two molecules. To distinguish between these possibilities pCAGArtTal1-Clo051 was cotransfected with the ArtTal1/TalRab2-reporter plasmid that contains only one ArtTal1 binding sequence. As shown in FIG. 10A the ArtTal1-Clo051 nuclease did not exhibit significant nuclease activity on the ArtTal1/TalRab2-reporter, indicating that two Clo051 nuclease domains must interact as a dimer to induce a DNA double-strand break. These results were confirmed with the TalRab2-Clo051 nuclease that acted on its corresponding TalRab2-reporter but not on the hybrid ArtTal1/TalRab2-reporter plasmid (FIG. 10A). As expected, the ArtTal1-FokI fusion protein did likewise not exhibit nuclease activity on the ArtTal1fTalRab2-reporter (FIG. 10B).

Next, we studied whether two Clo051 nuclease domains, that are fused to different arrays of TAL DNA binding elements, are also able to interact and to induce double-strand breaks. For this purpose the expression vectors pCAG-ArtTal1-Clo051 and pCAG-TalRab2-Clo051 were cotransfected together with the ArtTal1/TalRab2-reporter plasmid and the results compared to the cotransfection of pCAG-ArtTal1-Clo051 together with the ArtTal1/TalRab2-reporter. As shown in FIG. 10B, significant nuclease activity on the ArtTal1/TalRab2-reporter developed only by the coexpression of the ArtTal1- and TalRab2-Clo051 nucleases, indicating that Clo051 nuclease domains fused with different TAL arrays are able to interact and to induce a DNA double-strand break within a hybrid target region containing the recognition sequences of two distinguished TAL DNA binding arrays.

### EXAMPLE 2

Targeting of the Mouse Rab38 Gene in ES Cells and Zygotes with TAL-Clo051 Nucleases

### Construction of Rab38 Specific TAL-Clo051 Nucleases and a Targeting Vector

To demonstrate the functionality of TAL effector DNA-binding domain-nuclease fusion proteins in mammalian cells we designed a pair of fusion proteins that recognizes a DNA target sequence within the mouse Rab38 gene (FIG. 11). The two TAL effector DNA-binding domain-nuclease fusion proteins are intended to bind together to the bipartite target DNA region and to induce a double strand break in the spacer region of the target region to stimulate homologus recombination at the target locus in mammalian cells.

The mouse Rab38 gene encodes the RAB38 protein that is a member of a family of proteins known to play a crucial role in vesicular trafficking. In chocolate (cht) mutant mice a single nucleotide exchange at position 146 (G>T mutation) within the first exon of Rab38 leads to the replacement of glycine by valine at codon 19 (Loftus, S. K., et al., Proc Natl Acad Sci USA, 2002. 99(7): p. 4471-6). This amino acid replacement is located within the conserved GTP binding domain of RAB38 and impairs the sorting of the tyrosinase-related protein 1 (TYRP1) into the melanosomes of Rab38^{cht}/Rab38^{cht} melanocytes. TYRP1 is a melanosomal membrane glycoprotein, which functions both as a 5,6-Dihydroxyindol-2-carbonic-acid oxidase enzyme to produce melanin and as a provider of structural stability to tyrosinase in the melanogenic enzyme complex. TYRP1 is believed to transit from the trans-Golgi network to stage II melanosomes by means of clathrin-coated vesicles. The reduced amount of correctly located TYRP1 leads to an impairment of pigment production and the change of fur color from black to a chocolate-like brown color in Rab38^{cht}/Rab38^{cht} mice. Since mutations of genes needed for melanocyte function are known to cause oculocutaneous albinism (OCD), such as Hermansky-Pudlak syndrome in man, the Rab38 gene is a candidate locus in OCD patients.

We aimed to introduce a phenocopy of the chocolate mutation at codon 19 of Rab38 using a pair of TAL-nucleases (RabChtTal1- and RabChtTal2-Clo051) that each recognise a 14 bp target sequence located up- and downstream of a central 15 bp spacer sequence within exon 1 of the Rab38 gene (FIG. 11). To derive expression vectors for the RabChtTal1- and RabChtTal2-Clo051 nucleases synthetic coding regions for the DNA binding domains RabChtTal1 and RabChtTal2 composed of 14 TAL elements and the Clo051 nuclease domain were inserted into the pCAG-TAL-nuclease vector. The resulting plasmid pCAG-RabChtTal1-Clo051 (SEQ ID NO: 54) encodes the RabChtTal1-Clo051 fusion protein (SEQ ID NO: 55), and the plasmid pCAG-RabChtTal2-Clo051 (SEQ ID NO: 56) encodes the RabChtTal2-Clo051 fusion protein (SEQ ID NO: 57).

For the modification of the Rab38 gene by homologous recombination in fertilised oocytes we constructed the gene targeting vector pRab38-chtTAL (FIG. 12) (SEQ ID NO: 58), comprised of two homology regions encompassing 942 and 2788 bp of genomic sequence flanking exon1 of the mouse Rab38 gene (SEQ ID NO: 59). For this purpose the vectors 5′- and 3′-homology arms were amplified from the genomic BAC clone RPCI-421G2 (derived from the C57BL/6J genome, Imagenes GmbH, Berlin) using specific PCR primers. Within the sequence of codon 19 we introduced two nucleotide changes that modify codon 19 from the wildtype sequence GGT, coding for glycine, into GTA, coding for valine. This new chocolate mutation can be distinguished from the natural chocolate mutation, which exhibits only a single nucleotide exchange within codon 19 (GTT) coding for valine (Loftus, S. K., et al., Proc Natl Acad Sci USA, 2002. 99(7): p. 4471-6). Both chocolate mutant alleles can be further distinguished from the wildtype allele by restriction analysis since the mutations in codon 19 remove a recognition site for the restriction endonuclease BsaJI (FIG. 12). The recognition region for the TAL-nucleases is located downstream of codon 19 (FIG. 11). For the construction of the targeting vector 3′-homology region each 14 bp TAL fusion protein recognition sequence was further modified by the introduction of silent nucleotide changes that do not alter the RAB38 protein sequence (FIG. 12), in order to avoid the potential processing of the targeting vector by the Rab38 specific TAL-nucleases.

For the modification of the Rab38 gene by homologous recombination in mouse ES cells we modified the gene targeting vector pRab38-chtTAL (FIG. 12) by the insertion of a neomycin resistance gene as selection marker into spacer region of the TAL-nuclease recognition region, to derive the targeting vector pRab38-chtTAL-neo (SEQ ID NO: 60).

### Targeting of the Rab38 Gene in ES Cells and Zygotes

To demonstrate the utility of the RabChtTal1- and RabChtTal2-Clo051 proteins for gene targeting in mammalian cells (FIG. 3) we introduced the expression vectors or protein coding mRNA together with the pRab38-chtTAL-neo targeting vector into mouse ES cells or with the pRab38-chtTAL vector into fertilised mouse oocytes.

For targeting in ES cells we transfected IDG3.2 ES cells (Hitz, C. et al. Nucleic Acids Res. 35, e90, 2007) with linearised pRab38-chtTAL-neo targeting vector together with or without the TAL-nuclease expression plasmids pCAG-RabChtTal1- and pCAG-RabChtTal2-Clo051. The transfection, selection, expansion and genotyping of neomycin resistant ES cell clones was performed according to standard gene targeting procedures as described ((Nagy A, Gertsenstein M, Vintersten K, Behringer R., 2003. Manipulating the Mouse Embryo. Cold Spring Harbour, New York: Cold Spring Harbour Laboratory Press). The analysis of resistant ES cell clones revealed that the expression of the TAL-nucleases lead to a significantly increased rate of homologous recombination at the Rab38 gene in ES cells.

For microinjection into fertilised mouse oocytes the circular pRab38-chtTAL vector DNA was mixed with in vitro transcribed mRNA coding for RabChtTal1- and RabChtTal2-Clo051 proteins in injection buffer as described (Meyer, M., et al., Proc Natl Acad Sci USA. 107(34): p. 15022-6). TAL-nuclease mRNA is prepared from the linearised expression plasmids pCAG-RabChtTA11- and pCAG-RabChtTa12-Clo051

by in vitro transcription from the T7 promoter using the mMessage mMachine kit (Ambion) according to the manufacturers instructions. The mRNA is further modified by the addition of a poly-A tail using the Poly(A) tailing kit and purified with MegaClear columns from Ambion. Finally the mRNA is precipitated and resolved in injection buffer.

To isolate fertilised oocytes, males of the C57BL/6 strain are mated to super-ovulated females of the FVB strain. For super-ovulation three-week old FVB females are treated with 2.5 IU pregnant mares serum (PMS) 2 days before mating and with 2.5 IU Human chorionic gonadotropin (hCG) at the day of mating. Fertilised oocytes are isolated from the oviducts of plug positive females and microinjected in M2 medium (Sigma-Aldrich Inc Cat. No. M7167) with the TAL-nuclease mRNA and pRab38-chtTAL targeting vector preparation into one pronucleus and the cytoplasm following standard procedures (Nagy A, Gertsenstein M, Vintersten K, Behringer R., 2003. Manipulating the Mouse Embryo. Cold Spring Harbour, New York: Cold Spring Harbour Laboratory Press).

Upon microinjection the TAL-nuclease mRNAs are translated into proteins that induce a double-strand break at one or both Rab38 alleles in one or more cells of the developing embryo. This event stimulates the recombination of the pRab38-chtTAL targeting vector with a Rab38 allele via the homology regions present in the vector and leads to the site-specific insertion of the mutant codon 19 into the genome, resulting into a Rab38cIn allele bearing the chocolate mutation (FIG. 12). The microinjected zygotes were transferred into pseudopregnant females to allow their further development into live mice (Nagy A, Gertsenstein M, Vintersten K, Behringer R., 2003. Manipulating the Mouse Embryo. Cold Spring Harbour, New York: Cold Spring Harbour Laboratory Press). From the resulting offspring genomic DNA was extracted from tail tips to analyse for the presence of the desired homologous recombination event at the Rab38 locus by PCR. This analysis was performed by the PCR amplification of the genomic region encompassing exon1. The presence of a Rab38cht allele can be recognised upon digestion of the PCR products with BsaJl, since the Rab38^{cht} mutation at codon 19 leads to the removal of a BsaJI restriction site that is present in the wildtype sequence.

In one such experiment, mice derived from microinjected zygotes were analysed by a Rab38 PCR assay. Among this group most mice exhibited two alleles of the normal Rab38 wildtype genotype, whereas some individuals harboured one allele of the preplanned Rab38 chocolate mutation, as indicated by the absence of the BsaJl restriction site in exon 1

Taken together, it was possible to introduce a preplanned modification into the coding region of the Rab38 gene by TAL-Clo051 nuclease-assisted homologous recombination in mouse ES cells and fertilised oocytes.

### EXAMPLE 3

### Isolation of Hyperactive Clo051 Nuclease Mutants.

As shown in FIG. 13 the primary sequence of the Clo051 nuclease domain between the positions E389 and Y587 exhibits a unique distribution of the positively charged arginine (R) and lysine (K) residues and of negatively charged glutamate (E) and aspartate (D) residues. These residues constitute a three-dimensional landscape of charges within the Clo051 domain that determines the unique tertiary structure of this nuclease, as shown in the structural model in FIG. 6. Certain replacements of polar versus non-polar residues or of non-polar residues against polar residues, e.g. at the positions 423 and 446, alter the three-dimensional structure of the protein chain and can result into an increase of the nuclease activity.

Such amino acid replacements may be made by trial and error or may follow specific hypotheses on the structural and functional impact on the Clo051 nuclease domain. Alternatively, a large number of randomly mutagenised variants of the Clo051 nuclease domain coding region can be assembled in a library by mutagenic PCR. This library of mutant molecules can be tested for the presence of hyperactive nuclease variants by a phenotypic screening assay in yeast, mammalian or E. coli cells that is coupled to a functional nuclease readout, e.g. as described for the improvement of the FLP recombinase (Buchholz et al., Nat. Biotechnol. 16, 657-62, 1998).

Such a functional screen for improved nuclease variants can result into the replacement of e.g. the residue 423 from a serine to a proline and of the residue 446 from an arginine to a glutamate. Such variant molecules can prove a superior nuclease activity as compared to the Clo051 wildtype form.

### EXAMPLE 4

### Clo051 Nuclease Induced Recombination of Genomic Substrates in Human Cells

The action of Clo051 nuclease was further tested in human HEK293 cells on a genomic integrated reporter construct. For this purpose the ArtTal1 reporter plasmid (FIG. 7) was modified by the insertion of a hygromycin resistance gene into the plasmid backbone. In addition the β-galactosidase reading frame was fused with the coding region of the neomycin resistance gene, resulting in the reporter plasmid pCMV-Rab-Reporter(hygro) (SEQ ID NO: 61). To generate a cell line harboring the reporter construct in its genome, linearized reporter plasmid DNA was electroporated into human HEK 293 cells (ATCC #CRL-1573) (Graham F L, Smiley J, Russell W C, Nairn R., J. Gen. Virol. 36, 59-74, 1977) and hygromycin resistant clones were selected and isolated. One of the resistant clones, that showed no background activity of the reporter gene, 293ArtTal-Rep #2, was chosen for further work.

Next, one million reporter cells were transfected with 5 μg plasmid DNA of the Tal nuclease expression vector pCAG-ArtTal1-Clo051 (FIG. 4) or with 5 μg of the unrelated cloning vector pBluescript as negative control. Upon transfection the cells were seeded in duplicate wells of a 6-well tissue culture plate and cultured for two days before analysis was started. For analysis the transfected cells of each well were fixed for 10 minutes with 4% formaldehyde and incubated for 4 hours with X-Gal staining solution (5 mM K3(Felll(CN)6), 5 mM K4(Fell(CN)6), 2 mM MgCl2, 1 mg/ml X-Gal (5-bromo-chloro-3-indoyl-β-D-galactopyranosid). Recombined cells that express the reporter gene are visualized by an intracellular blue staining and were quantified on photographic images using the ImageJ software's cell counter function (available at the website with the address http://imagej.nih.gov/ij). As shown in FIG. 14A, transfection with the pBluescript control plasmid did not result in positive reporter cells (>0.1%, 0 positive cells of 1076 counted cells). In contrast, the transfection of pCAG-ArtTal-1 resulted into a substantial fraction of cells that recombined the reporter construct and express β-galactosidase (FIG. 14B). As quantified from photographic images, 42.7% of the reporter cells (227 positive cells of 531 counted cells) showed successful recombination as indicated by expression of the reporter gene. In conclusion, this result indicates that ArtTal1-Clo051 nuclease protein can efficiently process a target sequence located within mammalian genomic DNA.

What is claimed:

1. A nucleic acid molecule encoding
(I) a polypeptide having the activity of an endonuclease, which is selected from the group consisting of:
(a) a nucleic acid molecule encoding a polypeptide comprising or consisting of the amino acid sequence of SEQ ID NO: 1;
(b) a nucleic acid molecule comprising or consisting of the nucleotide sequence of SEQ ID NO: 2;
(c) a nucleic acid molecule encoding an endonuclease, the amino acid sequence of which is at least 70% identical to the amino acid sequence of SEQ ID NO: 1;
(d) a nucleic acid molecule comprising or consisting of a nucleotide sequence which is at least 50% identical to the nucleotide sequence of SEQ ID NO: 2;
(e) a nucleic acid molecule which is degenerate with respect to the nucleic acid molecule of (d); and
(f) a nucleic acid molecule corresponding to the nucleic acid molecule of any one of (a) to (e) wherein T is replaced by U; or
(II) a fragment of the polypeptide of (I) having the activity of an endonuclease.

2. The nucleic acid molecule of claim 1, wherein in (I)(c) in said amino acid sequence having at least 70% sequence identity to SEQ ID NO: 1 the amino acid residues P66, D67, D84 and/or K86 of SEQ ID NO: 1 are not modified.

3. The nucleic acid molecule of claim 1 further encoding a DNA-binding domain.

4. The nucleic acid molecule of claim 3, wherein the DNA-binding domain is a TAL effector motif of a TAL effector protein.

5. A vector comprising the nucleic acid molecule of claim 1.

6. A host cell comprising the nucleic acid molecule of claim 1.

7. A protein or fusion protein having the activity of an endonuclease encoded by the nucleic acid molecule of claim 1.

8. A method of modifying a target sequence in the genome of a eukaryotic cell, the method comprising the step of:
(a) introducing into said cell the nucleic acid molecule of claim 1, a vector of comprising the nucleic acid molecule of claim 1 or a protein or fusion protein having the activity of an endonuclease encoded by the nucleic acid molecule of claim 1.

9. The method of claim 8, wherein the modification of said target sequence is by homologous recombination with a donor nucleic acid sequence, further comprising the step:
(b) introducing a nucleic acid molecule into said cell, wherein said nucleic acid molecule comprises said donor nucleic acid sequence, wherein said donor DNA sequence is flanked upstream by a first flanking element and downstream by a second flanking element, wherein said first and second flanking element are different and wherein each of said first and second flanking element are homologous to a continuous DNA sequence on either side of the double-strand break introduced in (a) of claim 8 within said target sequence in the genome of said eukaryotic cell.

10. The method of claim 8, wherein said cell is analysed for successful modification of said target sequence in the genome.

11. The method of claim 8, wherein the cell is selected from the group consisting of a mammalian or vertebrate cell, a plant cell or a fungal cell.

12. The method of claim 8, wherein the cell is an oocyte.

13. A method of producing a non-human vertebrate or mammal carrying a modified target sequence in its genome, the method comprising transferring a cell produced by the method of claim 9 into a pseudo pregnant female host.

14. The method of claim 8, wherein the cell is selected from the group consisting of rodents, dogs, felides, primates, rabbits, pigs, cows, chickens, turkeys, pheasants, ducks, geese, quails, ostriches, emus, cassowaries and zebrafish.

15. A method of producing a protein or fusion protein having the activity of an endonuclease encoded by the nucleic acid molecule of claim 1 comprising the steps of: (a) culturing a host cell comprising the nucleic acid molecule of claim 1 and (b) isolating the produced protein or fusion protein.

16. A host cell comprising the vector of claim 5.

17. A protein or fusion protein having the activity of an endonuclease encoded by the vector of claim 5.
